SEQUENCE LISTING (325 sequences)

SEQ ID NO: 1 - protein, 586 aa, Homo sapiens
  misc_feature: SEQ ID NO 1 depicts the Sequence of Ezrin (Name of protein Ezrin; Synonyms p81, Cytovillin, Villin-2; Name of gene VIL2; from Homo sapiens (TaxID 9606); GI-Nummer GI 31282; UniProtKB/Swiss-Prot entry P15311)
MPKPINVRVTTMDAELEFAIQPNTTGKQLFDQVVKTIGLREVWYFGLHYVDNKGFPTWLKLDKKVSAQEVRKENPLQFKFRAKFYPEDVAEELIQDITQKLFFLQVKEGILSDEIYCPPETAVLLGSYAVQAKFGDYNKEVHKSGYLSSERLIPQRVMDQHKLTRDQWEDRIQVWHAEHRGMLKDNAMLEYLKIAQDLEMYGINYFEIKNKKGTDLWLGVDALGLNIYEKDDKLTPKIGFPWSEIRNISFNDKKFVIKPIDKKAPDFVFYAPRLRINKRILQLCMGNHELYMRRRKPDTIEVQQMKAQAREEKHQKQLERQQLETEKKRRETVEREKEQMMREKEELMLRLQDYEEKTKKAERELSEQIQRALQLEEERKRAQEEAERLEADRMAALRAKEELERQAVDQIKSQEQLAAELAEYTAKIALLEEARRRKEDEVEEWQHRAKEAQDDLVKTKEELHLVMTAPPPPPPPVYEPVSYHVQESLQDEGAEPTGYSAELSSEGIRDDRNEEKRITEAEKNERVQRQLLTLSSELSQARDENKRTHNDIIHNENMRQGRDKYKTLRQIRQGNTKQRIDEFEAL

SEQ ID NO: 2 - protein, 375 aa, Homo sapiens
  misc_feature: SEQ ID NO 2 shows the sequence of Serpin B5 (Name of protein Serpin B5 [Precursor]; Synonyms Maspin, Protease inhibitor 5; Name of gene SERPINB5; Synonyms PI5; from Homo sapiens (TaxID 9606); GI-Nummer GI 142377273; UniProtKB/ Swiss-Prot entry
MDALQLANSAFAVDLFKQLCEKEPLGNVLFSPICLSTSLSLAQVGAKGDTANEIGQVLHFENVKDIPFGFQTVTSDVNKLSSFYSLKLIKRLYVDKSLNLSTEFISSTKRPYAKELETVDFKDKLEETKGQINNSIKDLTDGHFENILADNSVNDQTKILVVNAAYFVGKWMKKFPESETKECPFRLNKTDTKPVQMMNMEATFCMGNIDSINCKIIELPFQNKHLSMFILLPKDVEDESTGLEKIEKQLNSESLSQWTNPSTMANAKVKLSIPKFKVEKMIDPKACLENLGLKHIFSEDTSDFSGMSETKGVALSNVIHKVCLEITEDGGDSIEVPGARILQHKDELNADHPFIYIIRHNKTRNIIFFGKFCSP

SEQ ID NO: 3 - protein, 198 aa, Homo sapiens
  misc_feature: SEQ ID NO 3 provides the sequence of Peroxiredoxin-2 (Name of protein Peroxiredoxin-2; Synonyms Thioredoxin peroxidase 1, etc; Name of gene PRDX2; Synonyms TDPX1; from Homo sapiens (TaxID 9606); GI-Nummer GI440307; UniProtKB/Swiss-Prot entry
MASGNARIGKPAPDFKATAVVDGAFKEVKLSDYKGKYVVLFFYPLDFTFVCPTEIIAFSNRAEDFRKLGCEVLGVSVDSQFTHLAWINTPRKEGGLGPLNIPLLADVTRRLSEDYGVLKTDEGIAYRGLFIIDGKGVLRQITVNDLPVGRSVDEALRLVQAFQYTDEHGEVCPAGWKPGSDTIKPNVDDSKEYFSKHN

SEQ ID NO: 4 - protein, 205 aa, Homo sapiens
  misc_feature: SEQ ID NO 4 shows the sequence of Heat shock protein beta-1 (Name of protein Heat shock protein beta-1; Synonyms HspB1, etc; Name of gene HSPB1; Synonyms HSP27; from Homo sapiens (TaxID 9606); GI-Nummer GI32477; UniProtKB/ Swiss-Prot entry
MTERRVPFSLLRGPSWDPFRDWYPHSRLFDQAFGLPRLPEEWSQWLGGSSWPGYVRPLPPAAIESPAVAAPAYSRALSRQLSSGVSEIRHTADRWRVSLDVNHFAPDELTVKTKDGVVEITGKHEERQDEHGYISRCFTRKYTLPPGVDPTQVSSSLSPEGTLTVEAPMPKLATQSNEITIPVTFESRAQLGGPEAAKSDETAAK

SEQ ID NO: 5 - protein, 37 aa, Homo sapiens
MGNHELYMRRRKPDTIEVQQMKAQAREEKHQKQLERQ

SEQ ID NO: 6 - protein, 25 aa, Homo sapiens
RKPDTIEVQQMKAQAREEKHQKQLE

SEQ ID NO: 7 - protein, 19 aa, Homo sapiens
QQMKAQAREEKHQKQLERQ

SEQ ID NO: 8 - protein, 14 aa, Homo sapiens
QAREEKHQKQLERQ

SEQ ID NO: 9 - protein, 67 aa, Homo sapiens
YEEKTKKAERELSEQIQRALQLEEERKRAQEEAERLEADRMAALRAKEELERQAVDQIKSQEQLAAE

SEQ ID NO: 10 - protein, 11 aa, Homo sapiens
EEKTKKAEREL

SEQ ID NO: 11 - protein, 68 aa, Homo sapiens
EKTKKAERELSEQIQRALQLEEERKRAQEEAERLEADRMAALRAKEELERQAVDQIKSQEQLAAELAE

SEQ ID NO: 12 - protein, 36 aa, Homo sapiens
LRAKEELERQAVDQIKSQEQLAAELAEYTAKIALLE

SEQ ID NO: 13 - protein, 70 aa, Homo sapiens
LRAKEELERQAVDQIKSQEQLAAELAEYTAKIALLEEARRRKEDEVEEWQHRAKEAQDDLVKTKEELHLV

SEQ ID NO: 14 - protein, 17 aa, Homo sapiens
EELERQAVDQIKSQEQL

SEQ ID NO: 15 - protein, 20 aa, Homo sapiens
EELERQAVDQIKSQEQLAAE

SEQ ID NO: 16 - protein, 23 aa, Homo sapiens
EELERQAVDQIKSQEQLAAELAE

SEQ ID NO: 17 - protein, 36 aa, Homo sapiens
EELERQAVDQIKSQEQLAAELAEYTAKIALLEEARR

SEQ ID NO: 18 - protein, 30 aa, Homo sapiens
LERQAVDQIKSQEQLAAELAEYTAKIALLE

SEQ ID NO: 19 - protein, 27 aa, Homo sapiens
LAAELAEYTAKIALLEEARRRKEDEVE

SEQ ID NO: 20 - protein, 18 aa, Homo sapiens
EYTAKIALLEEARRRKED

SEQ ID NO: 21 - protein, 25 aa, Homo sapiens
RKPDTIEVQQMKAQAREEKHQKQLE

SEQ ID NO: 22 - protein, 18 aa, Homo sapiens
EYTAKIALLEEARRRKED

SEQ ID NO: 23 - protein, 9 aa, Homo sapiens
TTMDAELEF

SEQ ID NO: 24 - protein, 15 aa, Homo sapiens
DRMAALRAKEELERQ

SEQ ID NO: 25 - protein, 13 aa, Homo sapiens
RAKEELERQAVDQ

SEQ ID NO: 26 - protein, 18 aa, Homo sapiens
SAELSSEGIRDDRNEEKR

SEQ ID NO: 27 - protein, 7 aa, Homo sapiens
KTKEELH

SEQ ID NO: 28 - protein, 18 aa, Homo sapiens
NIYEKDDKLTPKIGFPWS

SEQ ID NO: 29 - protein, 15 aa, Homo sapiens
KPINVRVTTMDAELE

SEQ ID NO: 30 - protein, 18 aa, Homo sapiens
NIYEKDDKLTPKIGFPWS

SEQ ID NO: 31 - protein, 25 aa, Homo sapiens
LRAKEELERQAVDQIKSQEQLAAEL

SEQ ID NO: 32 - protein, 11 aa, Homo sapiens
SQEQLAAELAE

SEQ ID NO: 33 - protein, 13 aa, Homo sapiens
QAREEKHQKQLER

SEQ ID NO: 34 - protein, 64 aa, Homo sapiens
KEELERQAVDQIKSQEQLAAELAEYTAKIALLEEARRRKEDEVEEWQHRAKEAQDDLVKTKEEL

SEQ ID NO: 35 - protein, 18 aa, Homo sapiens
WQHRAKEAQDDLVKTKEE

SEQ ID NO: 36 - protein, 13 aa, Homo sapiens
RAKEELERQAVDQ

SEQ ID NO: 37 - protein, 8 aa, Homo sapiens
QLAAELAE

SEQ ID NO: 38 - protein, 10 aa, Homo sapiens
QEQLAAELAE

SEQ ID NO: 39 - protein, 12 aa, Homo sapiens
RAKEELERQAVD

SEQ ID NO: 40 - protein, 10 aa, Homo sapiens
MAALRAKEEL

SEQ ID NO: 41 - protein, 11 aa, Homo sapiens
IAQDLEMYGIN

SEQ ID NO: 42 - protein, 9 aa, Homo sapiens
QEVRKENPL

SEQ ID NO: 43 - protein, 21 aa, Homo sapiens
ETAVLLGSYAVQAKFGDYNKE

SEQ ID NO: 44 - protein, 11 aa, Homo sapiens
EAQDDLVKTKE

SEQ ID NO: 45 - protein, 6 aa, Homo sapiens
KLFFLQ

SEQ ID NO: 46 - protein, 16 aa, Homo sapiens
RRKEDEVEEWQHRAKE

SEQ ID NO: 47 - protein, 16 aa, Homo sapiens
IALLEEARRRKEDEVE

SEQ ID NO: 48 - protein, 7 aa, Homo sapiens
KGFPTWL

SEQ ID NO: 49 - protein, 14 aa, Homo sapiens
QLFDQVVKTIGLRE

SEQ ID NO: 50 - protein, 8 aa, Homo sapiens
EELIQDIT

SEQ ID NO: 51 - protein, 19 aa, Homo sapiens
DQIKSQEQLAAELAEYTAK

SEQ ID NO: 52 - protein, 7 aa, Homo sapiens
DKYKTLR

SEQ ID NO: 53 - protein, 6 aa, Homo sapiens
WYFGLH

SEQ ID NO: 54 - protein, 14 aa, Homo sapiens
VLLGSYAVQAKFGD

SEQ ID NO: 55 - protein, 14 aa, Homo sapiens
EKDDKLTPKIGFPW

SEQ ID NO: 56 - protein, 11 aa, Homo sapiens
WLKLDKKVSAQ

SEQ ID NO: 57 - protein, 15 aa, Homo sapiens
DNAMLEYLKIAQDLE

SEQ ID NO: 58 - protein, 36 aa, Homo sapiens
IALLEEARRRKEDEVEEWQHRAKEAQDDLVKTKEEL

SEQ ID NO: 59 - protein, 15 aa, Homo sapiens
DNAMLEYLKIAQDLE

SEQ ID NO: 60 - protein, 7 aa, Homo sapiens
KEAQDDL

SEQ ID NO: 61 - protein, 9 aa, Homo sapiens
EDVAEELIQ

SEQ ID NO: 62 - protein, 19 aa, Homo sapiens
KLDKKVSAQEVRKENPLQF

SEQ ID NO: 63 - protein, 7 aa, Homo sapiens
DKYKTLR

SEQ ID NO: 64 - protein, 9 aa, Homo sapiens
EGILSDEIY

SEQ ID NO: 65 - protein, 11 aa, Homo sapiens
IAQDLEMYGIN

SEQ ID NO: 66 - protein, 7 aa, Homo sapiens
GTDLWLG

SEQ ID NO: 67 - protein, 7 aa, Homo sapiens
RTHNDII

SEQ ID NO: 68 - protein, 27 aa, Homo sapiens
AEELIQDITQKLFFLQVKEGILSDEIY

SEQ ID NO: 69 - protein, 7 aa, Homo sapiens
ERELSEQ

SEQ ID NO: 70 - protein, 9 aa, Homo sapiens
PIDKKAPDF

SEQ ID NO: 71 - protein, 7 aa, Homo sapiens
ILSDEIY

SEQ ID NO: 72 - protein, 28 aa, Homo sapiens
AQEVRKENPLQFKFRAKFYPEDVAEELI

SEQ ID NO: 73 - protein, 9 aa, Homo sapiens
KVSAQEVRK

SEQ ID NO: 74 - protein, 6 aa, Homo sapiens
AELAEY

SEQ ID NO: 75 - protein, 7 aa, Homo sapiens
GTDLWLG

SEQ ID NO: 76 - protein, 8 aa, Homo sapiens
TKKAEREL

SEQ ID NO: 77 - protein, 12 aa, Homo sapiens
PEDVAEELIQDI

SEQ ID NO: 78 - protein, 7 aa, Homo sapiens
VSYHVQE

SEQ ID NO: 79 - protein, 10 aa, Homo sapiens
PNTTGKQLFD

SEQ ID NO: 80 - protein, 6 aa, Homo sapiens
QRVMDQ

SEQ ID NO: 81 - protein, 7 aa, Homo sapiens
HNENMRQ

SEQ ID NO: 82 - protein, 12 aa, Homo sapiens
KVSAQEVRKENP

SEQ ID NO: 83 - protein, 11 aa, Homo sapiens
DVAEELIQDIT

SEQ ID NO: 84 - protein, 18 aa, Homo sapiens
HFENVKDIPFGFQTVTSD

SEQ ID NO: 85 - protein, 52 aa, Homo sapiens
KSLNLSTEFISSTKRPYAKELETVDFKDKLEETKGQINNSIKDLTDGHFENI

SEQ ID NO: 86 - protein, 14 aa, Homo sapiens
AKELETVDFKDKLE

SEQ ID NO: 87 - protein, 22 aa, Homo sapiens
ETVDFKDKLEETKGQINNSIKD

SEQ ID NO: 88 - protein, 18 aa, Homo sapiens
VDFKDKLEETKGQINNSI

SEQ ID NO: 89 - protein, 41 aa, Homo sapiens
FKDKLEETKGQINNSIKDLTDGHFENILADNSVNDQTKILV

SEQ ID NO: 90 - protein, 15 aa, Homo sapiens
DKLEETKGQINNSIK

SEQ ID NO: 91 - protein, 17 aa, Homo sapiens
DKLEETKGQINNSIKDL

SEQ ID NO: 92 - protein, 15 aa, Homo sapiens
LEETKGQINNSIKDL

SEQ ID NO: 93 - protein, 11 aa, Homo sapiens
DQTKILVVNAA

SEQ ID NO: 94 - protein, 8 aa, Homo sapiens
MMNMEATF

SEQ ID NO: 95 - protein, 39 aa, Homo sapiens
KIIELPFQNKHLSMFILLPKDVEDESTGLEKIEKQLNSE

SEQ ID NO: 96 - protein, 49 aa, Homo sapiens
KIIELPFQNKHLSMFILLPKDVEDESTGLEKIEKQLNSESQWTNPSTMA

SEQ ID NO: 97 - protein, 21 aa, Homo sapiens
KDVEDESTGLEKIEKQLNSES

SEQ ID NO: 98 - protein, 31 aa, Homo sapiens
KDVEDESTGLEKIEKQLNSESLSQWTNPSTM

SEQ ID NO: 99 - protein, 15 aa, Homo sapiens
ENLGLKHIFSEDTSD

SEQ ID NO: 100 - protein, 10 aa, Homo sapiens
KVCLEITEDG

SEQ ID NO: 101 - protein, 11 aa, Homo sapiens
ELNADHPFIYI

SEQ ID NO: 102 - protein, 11 aa, Homo sapiens
IRHNKTRNIIF

SEQ ID NO: 103 - protein, 51 aa, Homo sapiens
KIIELPFQNKHLSMFILLPKDVEDESTGLEKIEKQLNSESLSQWTNPSTMA

SEQ ID NO: 104 - protein, 15 aa, Homo sapiens
DKLEETKGQINNSIK

SEQ ID NO: 105 - protein, 8 aa, Homo sapiens
MMNMEATF

SEQ ID NO: 106 - protein, 41 aa, Homo sapiens; identical to SEQ ID NO: 89

SEQ ID NO: 107 - protein, 52 aa, Homo sapiens; identical to SEQ ID NO: 85

SEQ ID NO: 108 - protein, 15 aa, Homo sapiens
ENLGLKHIFSEDTSD

SEQ ID NO: 109 - protein, 14 aa, Homo sapiens
AKELETVDFKDKLE

SEQ ID NO: 110 - protein, 10 aa, Homo sapiens
KVCLEITEDG

SEQ ID NO: 111 - protein, 11 aa, Homo sapiens
DQTKILVVNAA

SEQ ID NO: 112 - protein, 22 aa, Homo sapiens
ETVDFKDKLEETKGQINNSIKD

SEQ ID NO: 113 - protein, 11 aa, Homo sapiens
ELNADHPFIYI

SEQ ID NO: 114 - protein, 12 aa, Homo sapiens
ELNADHPFIYII

SEQ ID NO: 115 - protein, 9 aa, Homo sapiens
LPFQNKHLS

SEQ ID NO: 116 - protein, 16 aa, Homo sapiens
ETKGQINNSIKDLTDG

SEQ ID NO: 117 - protein, 9 aa, Homo sapiens
EPLGNVLFS

SEQ ID NO: 118 - protein, 10 aa, Homo sapiens
HFENVKDIPF

SEQ ID NO: 119 - protein, 9 aa, Homo sapiens
MDALQLANS

SEQ ID NO: 120 - protein, 11 aa, Homo sapiens
FILLPKDVEDE

SEQ ID NO: 121 - protein, 21 aa, Homo sapiens
ILVVNAAYFVGKWMKKFPESE

SEQ ID NO: 122 - protein, 6 aa, Homo sapiens
DLFKQL

SEQ ID NO: 123 - protein, 8 aa, Homo sapiens
WTNPSTMA

SEQ ID NO: 124 - protein, 16 aa, Homo sapiens
KLEETKGQINNSIKDL

SEQ ID NO: 125 - protein, 9 aa, Homo sapiens
ALQLANSAF

SEQ ID NO: 126 - protein, 11 aa, Homo sapiens
NSAFAVDLFKQ

SEQ ID NO: 127 - protein, 17 aa, Homo sapiens
LGNVLFSPICLSTSLSL

SEQ ID NO: 128 - protein, 23 aa, Homo sapiens
MSETKGVALSNVIHKVCLEITED

SEQ ID NO: 129 - protein, 6 aa, Homo sapiens
ENILAD

SEQ ID NO: 130 - protein, 11 aa, Homo sapiens
KFKVEKMIDPK

SEQ ID NO: 131 - protein, 29 aa, Homo sapiens
VPGARILQHKDELNADHPFIYIIRHNKTR

SEQ ID NO: 132 - protein, 9 aa, Homo sapiens
RNIIFFGKF

SEQ ID NO: 133 - protein, 9 aa, Homo sapiens
DALQLANSA

SEQ ID NO: 134 - protein, 7 aa, Homo sapiens
DFKDKLE

SEQ ID NO: 135 - protein, 8 aa, Homo sapiens
GQINNSIK

SEQ ID NO: 136 - protein, 7 aa, Homo sapiens
FIYIIRH

SEQ ID NO: 137 - protein, 7 aa, Homo sapiens
LSMFILL

SEQ ID NO: 138 - protein, 16 aa, Homo sapiens
EIGQVLHFENVKDIPF

SEQ ID NO: 139 - protein, 6 aa, Homo sapiens
GDSIEV

SEQ ID NO: 140 - protein, 8 aa, Homo sapiens
VDKSLNLS

SEQ ID NO: 141 - protein, 11 aa, Homo sapiens
FENVKDIPFGF

SEQ ID NO: 142 - protein, 10 aa, Homo sapiens
LEKIEKQLNS

SEQ ID NO: 143 - protein, 7 aa, Homo sapiens
ILVVNAA

SEQ ID NO: 144 - protein, 7 aa, Homo sapiens
LSMFILL

SEQ ID NO: 145 - protein, 181 aa, Homo sapiens
IGKPAPDFKATAVVDGAFKEVKLSDYKGKYVVLFFYPLDFTFVCPTEIIAFSNRAEDFRKLGCEVLGVSVDSQFTHLAWINTPRKEGGLGPLNIPLLADVTRRLSEDYGVLKTDEGIAYRGLFIIDGKGVLRQITVNDLPVGRSVDEALRLVQAFQYTDEHGEVCPAGWKPGSDTIKPNVD

SEQ ID NO: 146 - protein, 18 aa, Homo sapiens
AFKEVKLSDYKGKYVVLF

SEQ ID NO: 147 - protein, 8 aa, Homo sapiens
EVKLSDYK

SEQ ID NO: 148 - protein, 13 aa, Homo sapiens
EVKLSDYKGKYVV

SEQ ID NO: 149 - protein, 14 aa, Homo sapiens
EVKLSDYKGKYVVL

SEQ ID NO: 150 - protein, 12 aa, Homo sapiens
YVVLFFYPLDFT

SEQ ID NO: 151 - protein, 9 aa, Homo sapiens
LFFYPLDFT

SEQ ID NO: 152 - protein, 10 aa, Homo sapiens
FSNRAEDFRK

SEQ ID NO: 153 - protein, 7 aa, Homo sapiens
LNIPLLA

SEQ ID NO: 154 - protein, 18 aa, Homo sapiens
VTRRLSEDYGVLKTDEGI

SEQ ID NO: 155 - protein, 16 aa, Homo sapiens
RRLSEDYGVLKTDEGI

SEQ ID NO: 156 - protein, 46 aa, Homo sapiens
LKTDEGIAYRGLFIIDGKGVLRQITVNDLPVGRSVDEALRLVQAFQ

SEQ ID NO: 157 - protein, 17 aa, Homo sapiens
GIAYRGLFIIDGKGVLR

SEQ ID NO: 158 - protein, 11 aa, Homo sapiens
IIDGKGVLRQI

SEQ ID NO: 159 - protein, 12 aa, Homo sapiens
VGRSVDEALRLV

SEQ ID NO: 160 - protein, 7 aa, Homo sapiens
EALRLVQ

SEQ ID NO: 161 - protein, 11 aa, Homo sapiens
NVDDSKEYFSK

SEQ ID NO: 162 - protein, 8 aa, Homo sapiens
KEYFSKHN

SEQ ID NO: 163 - protein, 181 aa, Homo sapiens; identical to SEQ ID NO: 145

SEQ ID NO: 164 - protein, 18 aa, Homo sapiens
AFKEVKLSDYKGKYVVLF

SEQ ID NO: 165 - protein, 14 aa, Homo sapiens
EVKLSDYKGKYVVL

SEQ ID NO: 166 - protein, 8 aa, Homo sapiens
EVKLSDYK

SEQ ID NO: 167 - protein, 8 aa, Homo sapiens
EVKLSDYK

SEQ ID NO: 168 - protein, 7 aa, Homo sapiens
EALRLVQ

SEQ ID NO: 169 - protein, 17 aa, Homo sapiens
GIAYRGLFIIDGKGVLR

SEQ ID NO: 170 - protein, 13 aa, Homo sapiens
EVKLSDYKGKYVV

SEQ ID NO: 171 - protein, 10 aa, Homo sapiens
FSNRAEDFRK

SEQ ID NO: 172 - protein, 46 aa, Homo sapiens; identical to SEQ ID NO: 156

SEQ ID NO: 173 - protein, 11 aa, Homo sapiens
IIDGKGVLRQI

SEQ ID NO: 174 - protein, 12 aa, Homo sapiens
YVVLFFYPLDFT

SEQ ID NO: 175 - protein, 11 aa, Homo sapiens
RQITVNDLPVG

SEQ ID NO: 176 - protein, 10 aa, Homo sapiens
RQITVNDLPV

SEQ ID NO: 177 - protein, 11 aa, Homo sapiens
RQITVNDLPVG

SEQ ID NO: 178 - protein, 6 aa, Homo sapiens
GLFIID

SEQ ID NO: 179 - protein, 12 aa, Homo sapiens
LLADVTRRLSED

SEQ ID NO: 180 - protein, 6 aa, Homo sapiens
VDEALR

SEQ ID NO: 181 - protein, 7 aa, Homo sapiens
DEALRLV

SEQ ID NO: 182 - protein, 9 aa, Homo sapiens
ITVNDLPVG

SEQ ID NO: 183 - protein, 9 aa, Homo sapiens
FKEVKLSDY

SEQ ID NO: 184 - protein, 6 aa, Homo sapiens
KGVLRQ

SEQ ID NO: 185 - protein, 7 aa, Homo sapiens
DEALRLV

SEQ ID NO: 186 - protein, 7 aa, Homo sapiens
IIAFSNR

SEQ ID NO: 187 - protein, 5 aa, Homo sapiens
YPLDF

SEQ ID NO: 188 - protein, 8 aa, Homo sapiens
YRGLFIID

SEQ ID NO: 189 - protein, 8 aa, Homo sapiens
DTIKPNVD

SEQ ID NO: 190 - protein, 7 aa, Homo sapiens
IIAFSNR

SEQ ID NO: 191 - protein, 5 aa, Homo sapiens
VLFFY

SEQ ID NO: 192 - protein, 5 aa, Homo sapiens
YPLDF

SEQ ID NO: 193 - protein, 8 aa, Homo sapiens
YRGLFIID

SEQ ID NO: 194 - protein, 7 aa, Homo sapiens
DGAFKEV

SEQ ID NO: 195 - protein, 6 aa, Homo sapiens
YFSKHN

SEQ ID NO: 196 - protein, 8 aa, Homo sapiens
SVDEALRL

SEQ ID NO: 197 - protein, 6 aa, Homo sapiens
NRAEDF

SEQ ID NO: 198 - protein, 12 aa, Homo sapiens
RKEGGLGPLNIP

SEQ ID NO: 199 - protein, 6 aa, Homo sapiens
YFSKHN

SEQ ID NO: 200 - protein, 8 aa, Homo sapiens
EVLGVSVD

SEQ ID NO: 201 - protein, 14 aa, Homo sapiens
EVLGVSVDSQFTHL

SEQ ID NO: 202 - protein, 8 aa, Homo sapiens
SVDEALRL

SEQ ID NO: 203 - protein, 8 aa, Homo sapiens
DTIKPNVD

SEQ ID NO: 204 - protein, 7 aa, Homo sapiens
PEEWSQW

SEQ ID NO: 205 - protein, 29 aa, Homo sapiens
RVSLDVNHFAPDELTVNHFAPDELTVKTK

SEQ ID NO: 206 - protein, 33 aa, Homo sapiens
RVSLDVNHFAPDELTVNHFAPDELTVKTKDGVV

SEQ ID NO: 207 - protein, 15 aa, Homo sapiens
RVSLDVNHFAPDELT

SEQ ID NO: 208 - protein, 14 aa, Homo sapiens
VNHFAPDELTVKTK

SEQ ID NO: 209 - protein, 20 aa, Homo sapiens
VNHFAPDELTVKTKDGVVEI

SEQ ID NO: 210 - protein, 24 aa, Homo sapiens
VKTKDGVVEITGKHEERQDEHGYI

SEQ ID NO: 211 - protein, 9 aa, Homo sapiens
TKDGVVEIT

SEQ ID NO: 212 - protein, 11 aa, Homo sapiens
EAAKSDETAAK

SEQ ID NO: 213 - protein, 18 aa, Homo sapiens
VNHFAPDELTVKTKDGVV

SEQ ID NO: 214 - protein, 15 aa, Homo sapiens
RVSLDVNHFAPDELT

SEQ ID NO: 215 - protein, 11 aa, Homo sapiens
EAAKSDETAAK

SEQ ID NO: 216 - protein, 14 aa, Homo sapiens
VNHFAPDELTVKTK

SEQ ID NO: 217 - protein, 24 aa, Homo sapiens
VKTKDGVVEITGKHEERQDEHGYI

SEQ ID NO: 218 - protein, 10 aa, Homo sapiens
RLPEEWSQWL

SEQ ID NO: 219 - protein, 14 aa, Homo sapiens
LATQSNEITIPVTF

SEQ ID NO: 220 - protein, 6 aa, Homo sapiens
YSRALS

SEQ ID NO: 221 - protein, 6 aa, Homo sapiens
LDVNHF

SEQ ID NO: 222 - protein, 15 aa, Homo sapiens
LSRQLSSGVSEIRHT

SEQ ID NO: 223 - protein, 6 aa, Homo sapiens
EEWSQW

SEQ ID NO: 224 - protein, 6 aa, Homo sapiens
DEHGYI

SEQ ID NO: 225 - protein, 10 aa, Homo sapiens
DELTVKTKDG

SEQ ID NO: 226 - protein, 9 aa, Homo sapiens
AKSDETAAK

SEQ ID NO: 227 - protein, 7 aa, Homo sapiens
SDETAAK

SEQ ID NO: 228 - protein, 9 aa, Homo sapiens
LTVKTKDGV

SEQ ID NO: 229 - protein, 6 aa, Homo sapiens
DVNHFA

SEQ ID NO: 230 - protein, 5 aa, Homo sapiens
DVNHF

SEQ ID NO: 231 - protein, 9 aa, Homo sapiens
TKDGVVEIT

SEQ ID NO: 232 - protein, 17 aa, Homo sapiens
VKTKDGVVEITGKHEER

SEQ ID NO: 233 - protein, 9 aa, Homo sapiens
YSLKLIKRL

SEQ ID NO: 234 - protein, 7 aa, Homo sapiens
YSKLIKL

SEQ ID NO: 235 - protein, 8 aa, Homo sapiens
YSKLIKHL

SEQ ID NO: 236 - protein, 9 aa, Homo sapiens
YSLKLIKRL

SEQ ID NO: 237 - protein, 5 aa, Homo sapiens
YLKIK

SEQ ID NO: 238 - protein, 7 aa, Homo sapiens
YALKIIK

SEQ ID NO: 239 - protein, 9 aa, Homo sapiens
GLEKIEKQL

SEQ ID NO: 240 - protein, 6 aa, Homo sapiens
GLEKIE

SEQ ID NO: 241 - protein, 6 aa, Homo sapiens
GLEKIE

SEQ ID NO: 242 - protein, 9 aa, Homo sapiens
GLEKIEKQL

SEQ ID NO: 243 - protein, 6 aa, Homo sapiens
LEIEKQ

SEQ ID NO: 244 - protein, 7 aa, Homo sapiens
LEEIEKQ

SEQ ID NO: 245 - protein, 12 aa, Homo sapiens
FCMGNIDSINCK

SEQ ID NO: 246 - protein, 4 aa, Homo sapiens
NIDS

SEQ ID NO: 247 - protein, 4 aa, Homo sapiens
NIDS

SEQ ID NO: 248 - protein, 9 aa, Homo sapiens
EYTAKIAEL

SEQ ID NO: 249 - protein, 7 aa, Homo sapiens
ETAKIAL

SEQ ID NO: 250 - protein, 8 aa, Homo sapiens
EVTAKIAL

SEQ ID NO: 251 - protein, 9 aa, Homo sapiens
EYTAKIAEL

SEQ ID NO: 252 - protein, 6 aa, Homo sapiens
EYAKIA

SEQ ID NO: 253 - protein, 7 aa, Homo sapiens
EYNAKIA

SEQ ID NO: 254 - protein, 9 aa, Homo sapiens
LSSELSQAR

SEQ ID NO: 255 - protein, 6 aa, Homo sapiens
SELQAR

SEQ ID NO: 256 - protein, 7 aa, Homo sapiens
SELTQAR

SEQ ID NO: 257 - protein, 9 aa, Homo sapiens
LSSELSQAR

SEQ ID NO: 258 - protein, 6 aa, Homo sapiens
LSSELS

SEQ ID NO: 259 - protein, 6 aa, Homo sapiens
LSSELS

SEQ ID NO: 260 - protein, 10 aa, Homo sapiens
LNIYEKDDKL

SEQ ID NO: 261 - protein, 7 aa, Homo sapiens
LNIEDKL

SEQ ID NO: 262 - protein, 10 aa, Homo sapiens
LNIFESQDKL

SEQ ID NO: 263 - protein, 10 aa, Homo sapiens
LNIYEKDDKL

SEQ ID NO: 264 - protein, 5 aa, Homo sapiens
IYEKD

SEQ ID NO: 265 - protein, 6 aa, Homo sapiens
DIYEKD

SEQ ID NO: 266 - protein, 9 aa, Homo sapiens
AKEELERQA

SEQ ID NO: 267 - protein, 7 aa, Homo sapiens
AKEELEQ

SEQ ID NO: 268 - protein, 8 aa, Homo sapiens
AKEELEKQ

SEQ ID NO: 269 - protein, 9 aa, Homo sapiens
AKEELERQA

SEQ ID NO: 270 - protein, 6 aa, Homo sapiens
EELERQ

SEQ ID NO: 271 - protein, 6 aa, Homo sapiens
EELERQ

SEQ ID NO: 272 - protein, 9 aa, Homo sapiens
AKEELERQA

SEQ ID NO: 273 - protein, 5 aa, Homo sapiens
KEERQ

SEQ ID NO: 274 - protein, 7 aa, Homo sapiens
KEQQERQ

SEQ ID NO: 275 - protein, 12 aa, Homo sapiens
AFKEVKLSDYKG

SEQ ID NO: 276 - protein, 7 aa, Homo sapiens
EVKLDYK

SEQ ID NO: 277 - protein, 8 aa, Homo sapiens
EVKLGDYK

SEQ ID NO: 278 - protein, 12 aa, Homo sapiens
AFKEVKLSDYKG

SEQ ID NO: 279 - protein, 7 aa, Homo sapiens
AKKLSDY

SEQ ID NO: 280 - protein, 10 aa, Homo sapiens
ALKQAKLSDY

SEQ ID NO: 281 - protein, 12 aa, Homo sapiens
EVKLSDYKGKYV

SEQ ID NO: 282 - protein, 7 aa, Homo sapiens
EVKLDYK

SEQ ID NO: 283 - protein, 8 aa, Homo sapiens
EVKLGDYK

SEQ ID NO: 284 - protein, 11 aa, Homo sapiens
EVKLSDYKGKY

SEQ ID NO: 285 - protein, 11 aa, Homo sapiens
EVKLSDYKGKY

SEQ ID NO: 286 - protein, 17 aa, Homo sapiens
EVEIPKLAFPSDYKGKY

SEQ ID NO: 287 - protein, 14 aa, Homo sapiens
SEIRHTADRWRVSL

SEQ ID NO: 288 - protein, 7 aa, Homo sapiens
SEIHADR

SEQ ID NO: 289 - protein, 9 aa, Homo sapiens
SEIEHIADR

SEQ ID NO: 290 - protein, 14 aa, Homo sapiens
SEIRHTADRWRVSL

SEQ ID NO: 291 - protein, 5 aa, Homo sapiens
DWRSL

SEQ ID NO: 292 - protein, 7 aa, Homo sapiens
DKWRASL

SEQ ID NO: 293 - protein, 11 aa, Homo sapiens
QLSSGVSEIRH

SEQ ID NO: 294 - protein, 6 aa, Homo sapiens
LSGVSE

SEQ ID NO: 295 - protein, 8 aa, Homo sapiens
HLSGGVSE

SEQ ID NO: 296 - protein, 11 aa, Homo sapiens
QLSSGVSEIRH

SEQ ID NO: 297 - protein, 8 aa, Homo sapiens
QLGVEIRH

SEQ ID NO: 298 - protein, 12 aa, Homo sapiens
QLGGGKVTEIRH

SEQ ID NO: 299 - protein, 11 aa, Homo sapiens
QLSSGVSEIRH

SEQ ID NO: 300 - protein, 5 aa, Homo sapiens
SEIRH

SEQ ID NO: 301 - protein, 5 aa, Homo sapiens
SEIRH

SEQ ID NO: 302 - protein, 11 aa, Homo sapiens
QLSSGVSEIRH

SEQ ID NO: 303 - protein, 4 aa, Homo sapiens
LSSG

SEQ ID NO: 304 - protein, 4 aa, Homo sapiens
LSSG

SEQ ID NO: 305 - protein, 11 aa, Homo sapiens
QLSSGVSEIRH

SEQ ID NO: 306 - protein, 7 aa, Homo sapiens
LSVSEIH

SEQ ID NO: 307 - protein, 8 aa, Homo sapiens
LSVSEIQH

SEQ ID NO: 308 - protein, 11 aa, Homo sapiens
QLSSGVSEIRH

SEQ ID NO: 309 - protein, 6 aa, Homo sapiens
SSGVSI

SEQ ID NO: 310 - protein, 7 aa, Homo sapiens
SSGVSAI

SEQ ID NO: 311 - protein, 12 aa, Streptococcus pyogenes
ALKQAKLSDYIG

SEQ ID NO: 312 - protein, 8 aa, Streptococcus pyogenes
AKKLSDYG

SEQ ID NO: 313 - protein, 12 aa, Streptococcus pyogenes
AFKEVKLSDYKG

SEQ ID NO: 314 - protein, 12 aa, Streptococcus pyogenes
EVKLGDYKNLVV

SEQ ID NO: 315 - protein, 8 aa, Streptococcus pyogenes
EVKLDYKV

SEQ ID NO: 316 - protein, 12 aa, Streptococcus pyogenes
EVKLSDYKGKYV

SEQ ID NO: 317 - protein, 14 aa, Streptococcus pyogenes
SEIEHIADRVGIIN

SEQ ID NO: 318 - protein, 7 aa, Streptococcus pyogenes
SEIHADR

SEQ ID NO: 319 - protein, 14 aa, Streptococcus pyogenes
SEIRHTADRWRVSL

SEQ ID NO: 320 - protein, 11 aa, Streptococcus pyogenes
ISSQILSEIEH

SEQ ID NO: 321 - protein, 6 aa, Streptococcus pyogenes
SSSEIH

SEQ ID NO: 322 - protein, 11 aa, Streptococcus pyogenes
QLSSGVSEIRH

SEQ ID NO: 323 - protein, 12 aa, Streptococcus pyogenes
YFYSNIDSCDIK

SEQ ID NO: 324 - protein, 4 aa, Streptococcus pyogenes
NIDS

SEQ ID NO: 325 - protein, 12 aa, Streptococcus pyogenes
FCMGNIDSINCK

(19) United States
(12) Patent Application Publication
(10) Pub. No.: US 2011/0033479 A1
(43) Pub. Date: Feb. 10, 2011

(54) PROTEINS EZRIN, SERPIN B5, PEROXIREDOXIN-2 AND HEAT SHOCK PROTEIN BETA-1 AS AUTOANTIGENS FOR PSORIASIS VULGARIS AND POSTSTREPTOCOCCAL DISEASES

(75) Inventors: Jörg C. Prinz, Munich (DE); Petra Besgen, Freising (DE); Paul Trommler, Neuried (DE)

Correspondence Address: McKeon Meunier Carlin & Curfman LLC, 817 W. Peachtree Street, Suite 900, Atlanta, GA, 30308, US

(73) Assignee: LUDWIG-MAXIMILIANS-UNIVERSITAT, Munich (DE)

(21) Appl. No.: 12/918,459

(22) PCT Filed: Sep. 29, 2008
(86) PCT No.: PCT/EP08/08267

§ 371 Date: Oct. 26, 2010

(30) Foreign Application Priority Data
Feb. 19, 2008 (EP) PCT/EP2008/001283

(51) Int. Cl. G01N 33/53; A61K 39/09; A61K 39/395; C07K 16/42

(52) U.S. Cl. 424/165.1; 436/501; 424/244.1; 422/430; 530/387.2

(57) ABSTRACT

The present invention relates to autoantigens selected from Ezrin, Serpin B5, Peroxiredoxin-2, heat shock protein β1, and/or peptides comprising at least 5 consecutive amino acid residues of one of these autoantigenic proteins having immunological activity, or fragments, variants, or epitopes thereof and/or mixtures of at least two of the proteins and/or peptides as diagnostic tool for the detection of streptococcal driven conditions or as therapeutical agent for the treatment of streptococcal driven conditions.

The present invention relates to selected autoantigens as diagnostic tools or therapeutical agents, compositions comprising them as well as processes for the detection of streptococcal driven conditions and test kits useful for these methods. The present invention also relates to the use of autoantigens or antiautoantigen antibodies for the treatment of streptococcal driven conditions.

Group A beta-haemolytic streptococci (GAS) are involved in infections of mammalian organisms and can induce several autoimmune disorders as secondary disorders. Examples for these GAS driven disorders are psoriasis, rheumatic fever and heart disease, post-streptococcal glomerulonephritis, or a variety of pediatric autoimmune neuropsychiatric disorders associated with streptococcal infections (PANDAS).

Infections by group A beta-haemolytic streptococci (GAS), particularly infections of mammalian organisms such as humans, typically lead to infectious diseases such as erypsipelas, phlegmon, angina, scarlet fever, rheumatic fever and sepsis. Therapies for these infections typically include administration of well known antibiotics. Such antibiotics include e.g. penicillin G and penicillin V, cephalosporin (a broad spectrum antibiotic derived from the class beta-lactam antibiotics obtained from fungi and related to penicillin (trade name Mefoxin)) and makrolides (an antibiotic with a lacton ring structure and a glucosidic bound amino sugar, e.g. clindamycin, erythromycin), etc. Therapies based on these antibiotics typically operate effectively and at least allow an amelioration or even an extinction of the infection as a primary disorder to be treated in the patient.

However, group A beta-haemolytic streptococci (GAS) may also lead to a number of secondary disorders, which are much more difficult to treat due to missing or inefficient therapies. Such group A beta-haemolytic streptococci (GAS) mediated secondary disorders include, inter alia, autoimmune diseases, such as rheumatic fever and heart disease, psoriasis, poststreptococcal glomerulonephritis or a variety of pediatric autoimmune neuropsychiatric disorders associated with streptococcal infections (PANDAS). In this context, the term PANDAS is typically used to describe a subset of children who have Obsessive Compulsive Disorder(s) (OCD) and/or tic disorders such as Tourette's Syndrome or Chorea minor, and in whom symptoms worsen following streptococcal infections such as “Strep throat” and Scarlet Fever.

In this context, rheumatic fever typically affects children or young adults, causing painful, inflamed joints and, in some cases, permanent damage to heart valves. Heart disease may include any extrinsic group A beta-haemolytic streptococci (GAS) mediated heart disorder, that affects the heart muscle or the blood vessels of the heart. Such extrinsic group A beta-haemolytic streptococci (GAS) mediated heart disorders include, e.g. extrinsic cardiomyopathies, in other words cardiomyopathies where the primary pathology is located outside the myocardium itself, in the present case due to an infection with group A beta-haemolytic streptococci (GAS). Such extrinsic group A beta-haemolytic streptococci (GAS) mediated heart disorders furthermore include disorders such as arrhythmia, coronary heart disease, coronary artery disease, dilated cardiomyopathy, heart attack, heart failure, hypertrophic cardiomyopathy, mitral regurgitation, pulmonary stenosis, etc. As a further example, (poststreptococcal) glomerulonephritis, also known as (poststreptococcal) glomerular nephritis, abbreviated GN, is typically characterized by inflammation of the glomeruli, or small blood vessels in the kidneys. It may be present with isolated hematuria and/or proteinuria (blood respiratory protein in the urine); or as a nephrotic syndrome, a nephritic syndrome, acute renal failure, or chronic renal failure. These syndroms are categorized into several different pathological patterns, which are broadly grouped into non-proliferative or proliferative types.

The main postulated mechanism for these sequelae is, according to present knowledge, a molecular mimicry, where due to a similarity between a pathogen and proteins from the host a cross-reactive immune reaction can be induced by the pathogen and can raise a T-cell response or antibodies against the host proteins whereby immune tolerance to autologous proteins is broken. In this context, common epitopes on streptococcal antigens and keratinocyte proteins have been demonstrated by cross-reactive monoclonal antibodies, and database searches have identified amino acid sequence homologies of streptococcal M proteins with keratin 6 or 17. However, immunogenicity was found only in selected cases. but seems to be not based on a universally applicable phenomenon.

Among the above autoimmune diseases psoriasis (vulgaris) is probably the longest known and most important group A beta-haemolytic streptococci (GAS) mediated secondary disorder. Psoriasis (vulgaris) is a common T-cell mediated autoimmune disease of the skin, which affects approximately 2% of Western populations. Psoriasis (vulgaris) is a disease which typically affects the skin and the joints. Psoriasis (vulgaris) typically appears with heavily scaling red inflammatory plaques/red scaly patches that may cover large areas of the body. The scaly patches caused by psoriasis (vulgaris), also called psoriatic plaques, are areas of inflammation and excessive skin production. The skin rapidly accumulates at these sites and takes a silvery-white appearance. Plaques frequently occur on the skin of the elbows and knees, but can affect any area including the scalp and genitals. Disease manifestation of psoriasis (vulgaris) is influenced by several predisposing or protective gene loci and by environmental factors, with streptococcal throat infections being the most common trigger of first psoriasis onset or exacerbations. Multiple clinical observations and experimental findings using animal models have clearly established that the antigen-specific activation of T cells is central to induction and maintenance of psoriatic inflammation. The symptoms of psoriasis (vulgaris) can manifest in a variety of forms. Variants include different subtypes of psoriasis (vulgaris) such as exanthematic guttate psoriasis, chronic plaque psoriasis, erythrodermic psoriasis, pustular psoriasis, and psoriatic arthritis, etc.

Erythrodermic psoriasis as one form of psoriasis involves the widespread inflammation and exfoliation of the skin over most of the body surface. It may be accompanied by severe itching, swelling and pain. It is often the result of an exacerbation of unstable psoriasis vulgaris (plaque psoriasis), particularly following the abrupt withdrawal of systemic treatment. This form of psoriasis can be fatal, as the extreme inflammation and exfoliation disrupt the body's ability to regulate temperature and for the skin to perform barrier functions.

Psoriasis pustulosa appears as raised bumps that are filled with non-infectious pus (pustules). The skin under and surrounding these pustules is red and tender. Pustular psoriasis can be localized, commonly, to the hands and feet (palmoplantar pustulosis), or generalized with widespread patches occurring randomly on any part of the body.

Psoriatic arthritis as a further form of psoriasis involves joint and connective tissue inflammation. Psoriatic arthritis can affect any joint but is most common in the joints of the fingers and toes. This can result in a sausage-shaped swelling of the fingers and toes known as dactylis. Psoriatic arthritis can also affect the hips, knees and spine (spondylitis). About 10-15% of people who have psoriasis also have psoriatic arthritis.

Other forms of psoriasis include e.g. drug mediated psoriasis, which may occur due to administration of beta-blockers, ACE-supressants, lithium containing drugs, anti-malaria agents such as chloroquine, or interferon; nail psoriasis, which produces a variety of changes in the appearance of finger and toe nails; (exanthematic) guttate psoriasis, which is characterized by numerous small oval (teardrop-shaped) spots; and flexural psoriasis (inverse psoriasis), which appears as smooth inflamed patches of skin, particularly around the genitals (between the thigh and groin), the armpits, under an overweight stomach (pannus), and under the breasts (inframmary fold).

It is assumed that in general these forms of psoriasis may be caused by molecular mimicry as described above for group A beta-haemolytic streptococci (GAS) related secondary disorders in general. This concept is supported by the observation that lesional psoriatic T-cell clones were also identified within the tonsils of patients with streptococcal-driven psoriasis, constituting a link between streptococcal angina and psoriatic inflammation. Thus, psoriasis may actually represent a T-cell mediated autoimmune disease resulting from a cross-reactive immune response based on molecular mimicry.

However, even though the mechanisms of group A beta-haemolytic streptococci (GAS) related secondary disorders, particularly of psoriasis, now may have been investigated, diagnosis as well as therapy of those disorders still remain an unsolved challenge.

Diagnosis of psoriasis is usually based on the appearance of the skin. Up to date, there are no special blood tests or diagnostic procedures for psoriasis. Sometimes a skin biopsy, or scraping, may be needed to rule out other disorders and to confirm the diagnosis. Therefore, skin from a biopsy will show clubbed rete pegs if positive for psoriasis. Another diagnosis of psoriasis is possible, when the plaques are scraped and pinpoint bleeding from the skin below occurs (Auspitz's sign). However, any of these diagnosis methods requires a clear pathological finding, i.e. a group A beta-haemolytic streptococci (GAS) mediated secondary disorder such as psoriasis is usually present in an advanced state, when carrying out the diagnosis. This, of course, causes difficulties when early treatment of these group A beta-haemolytic streptococci (GAS) mediated secondary disorders is envisaged. Late diagnosis is also problematic for other reasons, e.g., when administration of higher dosages of specific drugs during elevated stages of disease may become necessary.

With regard to treatment, there are many therapeutic approaches available for different forms of psoriasis. However, due to its chronic recurrent nature psoriasis is still a challenge to treat and there remains a need in the art to provide further and more efficient treatment strategies. Since there can be substantial variation between individuals in the effectiveness of specific psoriasis therapies with respect to its location, extent and severity, and the patient's age, gender, quality of life, comorbidities, and attitude toward risks associated with the treatment are also taken into consideration, and most dermatologists use a trial-and-error approach to find the most appropriate treatment for their patient.

Typical approaches to treat in particular the above mentioned forms of psoriasis include e.g. medical therapies, topical therapies, phototherapy, photochemotherapy, or systemic therapies, as well as further alternative therapies. When treating a patient, typically administration of medicaments will be considered first. Thereby, those medications with the least potential for adverse reactions are preferentially employed. However, if the treatment goal is not achieved thereby, therapies with greater potential toxicity may be used (psoriasis treatment ladder), e.g. medications with significant toxicity are reserved for severe unresponsive psoriasis. As a first step, typically medicated ointments or creams are applied to the skin as a topical treatment. If topical treatment fails to achieve the desired goal, the next step would be to expose the skin to a phototherapy, e.g. ultraviolet (UV) radiation. The third step involves systemic treatment, wherein medicaments are administered, e.g. as a tablet or by injection. It was observed, that over time psoriasis can become resistant to a specific therapy. Treatments may thus be periodically changed according to a so called treatment rotation to prevent resistance developing (tachyphylaxis) and to reduce the chance of adverse reactions. Additonally or alternatively, other therapies may also be applied and typically include antibiotics, climatotherapy, etc., which are not indicated in routine treatment of psoriasis.

Summarizing the above, there are not yet any diagnosis methods available, which allow an early detection of psoriasis, or any therapy methods, which allow an efficient treatment of any of the above forms of psoriasis.

Generally group A beta-haemolytic streptococci (GAS) are involved in secondary disorders, particularly autoimmune disorders. Examples for GAS driven disorders are rheumatic fever and heart disease, post-streptococcal glumerulonephritis or a variety of pediatric autoimmune neuropsychiatric disorders associated with streptococcal infections (PANDAS).

Accordingly there is an urgent need in the art to provide methods and diagnostic tools which allow an early detection and treatment of GAS driven disorders like psoriasis or other autoimmune diseases. A further object of the present invention is the provision of compositions that can be used to detect or treat GAS driven diseases and kits useful therefore. Furthermore, there is a need for a method to detect a GAS driven disease and to determine a peptide useful for hyposensitizationor desensitization as specific immune therapy in the case of a GAS mediated autoimmune disease.

All these objects are solved by the subject matter of the attached claims. The present invention provides autoantigens selected from Ezrin, Serpin B5, Peroxiredoxin-2, Heat shock protein beta-1, or fragments, variants, or epitopes therof and/or peptides comprising at least 5 consecutive amino acid residues of one of these autoantigenic proteins having immunologic acitivity, and/or mixtures of at least two of the proteins and/or peptides as diagnostic tool for the detection of streptococcal driven conditions or as therapeutical agent for the treatment of streptococcal driven conditions. In the following the term “autoantigen” shall comprise at least one protein selected from Ezrin, Serpin B5, Peroxiredoxin-2, Heat shock protein beta-1, or fragments, variants, or epitopes therof and/or peptides comprising at least 5 consecutive amino acid residues of one of these autoantigehic proteins having immunologic acitivity, unless the context defines otherwise. Mixtures of at least two of the proteins and/or peptides and/or fragments, variants or epitopes are also subsumed under the term “autoantigen” unless the context refers to a single compound.

It has surprisingly been found that the proteins Ezrin, Serpin, Peroxiredoxin, and Heat shock protein beta-1 have sequences in common with structures of GAS. The inventor found that the proteins Ezrin, Serpin, Peroxiredoxin, and Heat shock protein beta-1 and, therefore, can be targets for antibodies that have been raised against GAS. The inventor concluded that GAS produces peptides or proteins that are molecular mimicries of at least one of Ezrin, Serpin, Peroxiredoxin, and Heat shock protein beta-1. Therefore, an immune response elicited by GAS can produce an autoimmune response against at least one of Ezrin, Serpin, Peroxiredoxin, and Heat shock protein beta-1. The immune reactions can be B cell mediated, T cell mediated or both. Based on this knowledge diagnostic tools as well as therapeutic agents for treating GAS driven conditions are provided.

It was found that these autoantigens can elicit T-cell mediated responses as well as B-cell mediated responses. The inventor found that with the present invention it is possible to determine a T-cell response and/or a B-cell response and with the result of this determination it is possible to make conclusions regarding the underlying condition or primary infection in an early stage. Moreover, with the knowledge of the underlying principle it is possible to find a better and more effective treatment.

According to a first aspect the present invention is concerned with autoantigens as defined above as diagnostic tools for the detection of streptococcal driven conditions. It has been found that Peroxiredoxin and Serpin induced a significantly increased T-cell stimulation in psoriasis patients compared to healthy controls. The reaction with Heat shock protein beta-1 was more significant for HLA-Cw6 negative patients and Ezrin also resulted in a higher T-cell activation in psoriasis patients than in healthy controls. Thus, at least one of these autoantigens or a fragment or derivative thereof or a mixture is useful as diagnostic tool for the detection of GAS driven conditions. At least one of these autoantigens or a peptide, fragment, variant, epitope or derivative of one of the before-mentioned compounds can be used to determine if an immune response has been elicited in a patient. If the presence of antiautoantigen antibodies can be detected this is an indication to a streptococcal driven condition. To detect antibodies reactive with these autoantigens the whole protein can be used or a reactive part thereof. A peptide comprising at least 5 consecutive amino acid residues of one of Ezrin, Serpin B5, Peroxiredoxin-2, or Heat shock protein beta-1 that is reactive with an antibody for which reactivity with one of the above-mentioned proteins has been shown, is also useful and is within the scope of the present invention.

Moreover, an autoantigen of the present invention or a fragment or derivate thereof that is reactive with antibodies against one of Ezrin, Serpin, Peroxiredoxin, or Heat shock protein beta-1, can also be used as a therapeutical agent to treat a condition that has been found to be a secondary streptococcal condition. Moreover, it is possible to prepare a derivative of the autoantigen or a peptide thereof wherein the derivatization is such that the molecule can block anti-autoantigen antibodies as it is known to the skilled artisan. It is also possible to immobilize autoantigens or reactive parts thereof and to allow antibodies present in a body fluid to bind to the autoantigen or a part thereof to thereby remove the antibodies from the body fluid.

By the unexpected finding that proteins Ezrin, Serpin B5, Peroxiredoxin-2, and Heat shock protein beta-1 are associated with GAS-mediated secondary disorders it is possible to detect and treat autoimmune reactions caused by GAS in the body.

Thus, according to a first embodiment the present invention provides an autoantigen selected from Ezrin, Serpin, Peroxiredoxin, and Heat shock protein beta-1 or a variant, a fragment or an epitope of those autoantigenic proteins for the above mentioned use. It has been found that the T-cell response against these four autoantigens that are correlated with GAS driven conditions, may differentiate dependent from blood groups and other variables. Therefore, it is preferred to use at least two autoantigens selected from the above mentioned group. Moreover it is possible to use at least two different peptides comprising at least 5 consecutive amino acid residues that are epitopes of one of these autoantigenic proteins or a mixture of autoantigens and peptides. Combinations of proteins and peptides, of peptides comprising different parts of one of these autoantigenic proteins or mixtures of peptides comprising parts of different autoantigens can be used. The more reactive compounds are present, the more detailed the analysis can be.

It has been found that the autoantigens are reactive in the following order: Ezrin, Serpin, Peroxiredoxin, and Heat shock protein beta-1. Therefore, a combination of Ezrin with one of the other autoantigens or parts thereof is most preferred.

Thus, an autoantigen or a composition for use in the present invention can comprise one of the following combinations of the autoantigenic proteins:
Ezrin, or
Serpin B5, or
Ezrin and Serpin B5.

and/or peptides comprising at least 5 consecutive amino acid residues of one of these autoantigenic proteins having immunological activity, or fragments, variants, or epitopes thereof

Even more preferably, the autoantigen or composition of the invention comprises one of the following combinations of the abovementioned autoantigenicproteins:
Ezrin, or
Serpin B5, or
Peroxiredoxin-2, or
Ezrin and Serpin B5, or
Ezrin and Peroxiredoxin-2, or
Serpin B5 and Peroxiredoxin-2, or
Ezrin and Serpin B5 and Peroxiredoxin-2.

and/or peptides comprising at least 5 consecutive amino acid residues of one of these autoantigenic proteins having innumological activity, or fragments, variants, or epitopes thereof

Most preferably, the autoantigen or composition of the present invention comprises one of the following combinations of the autoantigen:
Ezrin, or
Serpin B5, or
Peroxiredoxin-2, or
Heat shock protein beta-1, or
Ezrin and Serpin B5, or
Ezrin and Peroxiredoxin-2, or
Ezrin and Heat shock protein beta-1, or
Serpin B5 and Peroxiredoxin-2, or
Serpin B5 and Heat shock protein beta-1, or
Peroxiredoxin-2 and Heat shock protein beta-1, or
Ezrin and Serpin B5 and Peroxiredoxin-2, or
Ezrin and Serpin B5 and Heat shock protein beta-1, or
Ezrin and Peroxiredoxin-2 and Heat shock protein beta-1, or
Serpin B5 and Peroxiredoxin-2 and Heat shock protein beta-1, or
Ezrin and Serpin B5 and Peroxiredoxin-2 and Heat shock protein beta-1.

and/or peptides comprising at least 5 consecutive amino acid residues of one of these autoantigenic proteins having immunological activity, or fragments, variants, or epitopes thereof

In the context of the present invention, the autoantigenic protein Ezrin may be synonymously called p81, cytovillin or villin-2. It is a member of the Ezrin, Radixin, Moesin (ERM) family and acts as a linker protein located between cell surface receptors, adhesion molecules, and actin cytoskeleton. Ezrin was originally identified as a component of structures at the cell surface that contain an actin cytoskeleton, such as microvilli and membrane ruffles, and as a substrate of specific protein tyrosine kinases. It is highly enriched in microvilli on the apical side of polarized epithelial cells. The activity of Ezrin is regulated by intramolecular interactions between N- and C-terminal ERM association domains, wherein phosphorylation at threonine 567 is regarded as a critical regulator of Ezrin function allowing the active protein to link target molecules to the actin cytoskeleton. Ezrin tyrosine phosphorylation can also be induced by EGF, PDGF and HGF stimulation. Ezrin also interacts with other proteins, particularly PI3-K protein kinase A and Rho. Ezrin is a relevant protein in kidney function. Ezrin is expressed in the filtration barrier in the kidney glomerulus, where the filtration slits between podocyte foot processes are believed to be maintained by restriction of podocalyxin to the apical membrane by ezrin. The structural integrity of podocytes depends on cytoskeleton-associated proteins, particularly podocalyxin, synaptopodin and ezrin. Podocytes have essential roles in the formation and maintenance of the glomerular filtration barrier of the kidney. Ezrin is expressed in cerebral cortex, basal ganglia, hippocampus, hypophysis, and optic nerve. Preferential expression was found in astrocytes of hippocampus, frontal cortex, thalamus, parahippocampal cortex, amygdala, insula, and corpus callosum. Ezrin is furthermore weakly expressed in brain stem and diencephalon. Stronger expression was detected in gray matter of frontal lobe compared to white matter (at protein level). Ezrin represents a component of the microvilli of intestinal epithelial cells. It was not detected in neurons in most tissues studied. In the context of the present invention, the autoantigenic protein Ezrin comprises preferably a sequence as deposited under UniProtKB/Swiss-Prot entry P15311, GI number GI 31282 (gene name VIL2 from Homo sapiens (TaxID: 9606)). More preferably, the autoantigenic protein Ezrin comprises a sequence according to SEQ ID NO: 1 or a sequence showing an identity of at least about 60%, preferably of at least about 70% or about 80%, even more preferably of at least about 90% or about 95%, and most preferably an identity of at least about 99% with a sequence according to SEQ ID NO: 1. In another preferred embodiment the autoantigen is a variant or fragment of the above defined protein Ezrin.

Alternatively or additionally, the autoantigen based on Ezrin as defined herein may be provided in the form of at least one epitope of Ezrin. In this context, epitopes of autoantigenic proteins as defined herein, are typically a region within the autoantigenic protein, its variants or fragments as defined herein, that has the potential to give rise to an antibody response or are being recognized by T-cell antigen receptors and induce activation and expansion of antigen-specific T-cell populations (i.e. the autoantigenic protein, its variants or fragments as defined herein as well as an epitope thereof may comprise antigenic properties). More preferably, epitopes may be defined on the basis of the primary, secondary, or tertiary structure of the autoantigenic protein, e.g. Ezrin, Serpin B5, Peroxiredoxin-2 or Heat shock protein beta-1, its variants or fragments, and, consequently, may be exposed or hidden within the molecule. Each of the possible epitopes of such an autoantigenic protein in turn may be capable of generating a clone of B lymphocytes which produces an antibody against this epitope or induce an antigen-specific T-cell activation.

In a preferred embodiment the autoantigen comprises at least one epitope of Ezrin that has a common sequence with a GAS epitope.

In the context of the present invention, an epitope typically comprises a length of about 5 to 35 contiguous amino acids or even more of a sequence as defined herein, more preferably a length of about 5 to 20 contiguous amino acids of a sequence as defined herein, and most preferably a length of about 5 to 15 contiguous amino acids of a protein sequence as defined herein. As the case may be, such sequences, however, may also be longer, such as 30 to 100 or even 200 contiguous amino acids. In the case of Ezrin, an epitope preferably comprises a sequence having a length of about 5 to 35 contiguous amino acids or more, more preferably a length of about 5 to 20 contiguous amino acids, and most preferably a length of about 5 to 15 contiguous amino acids of the sequence according to SEQ ID NO: 1.

According to a specifically preferred embodiment, epitopes from Ezrin are selected from the following sequences according to SEQ ID NOs: 5-83:

| | | aa position on | |
|---|---|---|---|
| SEQ ID NO: | aa sequence of epitope | SEQ ID NO: 1: | homologies to specific gene |
| 5 | MGNHELYMRRRKPDTIEVQQ | 285-321 | GENE ID: 4066824 emm1-0 |
| | MKAQAREEKHQKQLERQ | | gb\|AAQ94528.1\|; gb\|AAQ94513.1\|; |
| | | | gb\|AAQ94548.1\|; gb\|AAQ73227.1\|; |
| | | | gb\|AAQ73207.1\|; gb\|AAY30321.1\|; |
| | | | gb\|AAN64673.1\|; gb\|AAD13149.1\|; |
| | | | gb\|AAD13150.1\|; gb\|AAN64675.1\|; |
| | | | gb\|AAC64110.1\|; |
| | | | emb\|CAA33269.1\|; |
| 6 | RKPDTIEVQQMKAQAREEKH | 295-319 | GENE ID: 4061023 |
| | QKQLE | | MGAS9429_Spy0359; |
| | | | GENE ID: 900682 SPy_0433 |
| 7 | QQMKAQAREEKHQKQLERQ | 303-321 | gb\|AAF86503.1\|AF283810_1; |
| | | | gb\|AAB06613.1\|; |
| 8 | QAREEKHQKQLERQ | 308-321 | gb\|ABO71772.1\|; |
| | | | emb\|CAE51956.2\|; |
| | | | gb\|AAQ94514.1\|; gb\|AAQ73236.1\|; |
| | | | gb\|AAQ73228.1\|; |
| | | | gb\|AAQ23117.1\|; gb\|AAN04082.1\|; |
| | | | gb\|AAY30318.1\|; |
| | | | gb\|AAF13404.1\|AF191300_1; |
| | | | gb\|AAN64677.1\|; gb\|AAC27083.2\|; |
| | | | gb\|AAB51153.1\|; gb\|AAN64683.1\|; |
| | | | gb\|AAC05162.1\|; gb\|AAB17102.1\|; |
| | | | gb\|AAC06230.1\|; |
| 9 | YEEKTKKAERELSEQIQRALQ | 354-420 | gb\|AAQ94546.1\|; gb\|AAQ94503.1\|; |
| | LEEERKRAQEEAERLEADRM | | gb\|AAQ94534.1\|; |
| | AALRAKEELERQAVDQIKSQE | | gb\|AAQ94496.1;\| gb\|AAQ94500.1\|; |
| | QLAAE | | gb\|AAQ94517.1\|; |
| | | | gb\|AAQ94539.1\|; gb\|AAQ94504.1\|; |
| | | | gb\|AAQ94538.1\|; |
| | | | gb\|AAQ94502.1;\| gb\|AAQ73218.1\|; |
| | | | gb\|AAQ73226.1\|; gb\|AAQ73229:1\|; |
| | | | gb\|AAQ73234.1\|; gb\|AAM52326.1\|; |
| | | | emb\|CAA53379.1\|; |
| | | | gb\|AAY30312.1\|; gb\|AAK11620.1\|; |
| | | | GENE ID: 3574431 M28_Spy1702; |
| 10 | EEKTKKAEREL | 355-365 | gb\|ABF82024.1\| |
| 11 | EKTKKAERELSEQIQRALQLE | 356-423 | gb\|AAQ73235.1\| |
| | EERKRAQEEAERLEADRMAA | | |
| | LRAKEELERQAVDQIKSQEQL | | |
| | AAELAE | | |
| 12 | LRAKEELERQAVDQIKSQEQL | 397-432 | gb\|AAN46661.1\| |
| | AAELAEYTAKIALLE | | |
| 13 | LRAKEELERQAVDQIKSQEQL | 397-466 | gb\|ABF82024.1;\|embCAA50980.1\| |
| | AAELAEYTAKIALLEEARRRKE | | |
| | DEVEEWQHRAKEAQDDLVKT | | |
| | KEELHLV | | |
| 14 | EELERQAVDQIKSQEQL | 401-417 | gb\|AAC78099.1\|; gb\|AAF71613.1\|; |
| | | | emb\|CAE51957.2\|; |
| | | | GENE ID: 4063046 |
| | | | MGAS10270_Spy1784; |
| 15 | EELERQAVDQIKSQEQLAAE | 401-420 | gb\|ABP01573.1\|; gb\|AAN64672.1\|; |
| | | | gb\|AAQ73233.1\|; gb\|AAM52325.1\|; |
| | | | gb\|AAY30309.1\|; gb\|AAY30316.1\|; |
| | | | gb\|AAF44783.1\|; gb\|AAF71609.1\|; |
| | | | gb\|AAG01353.1\|AF293886_1; |
| | | | gb\|AAN64685.1\|; |
| | | | gb\|AAD55744.1\|AF183965_1; |
| | | | gb\|AAF71610.1\|; |
| | | | gb\|AAD53111.1\|AF176247_1; |
| | | | gb\|AAF71612.1\|; gb\|AAF71614.1\|; |
| | | | gb\|AAD34608.1\|AF149049_1; |
| | | | gb\|ABU63692.1\|; gb\|ABO71767.1\|; |
| | | | gb\|ABF82021.1\|; gb\|AAC79688.1\|; |
| | | | gb\|AAN64684.1\|; gb\|ABF82023.1\|; |
| | | | gb\|ABF82022.1\|; emb\|CAL48337.1\|; |
| | | | gb\|ABO71769.1\|; gb\|AAY20968.1\|; |
| | | | gb\|ABO71779.1\|; gb\|ABO71778.1\|; |
| 16 | EELERQAVDQIKSQEQLAAEL | 401-423 | gbA\|AA50855.1\| |
| | AE | | |
| 17 | EELERQAVDQIKSQEQLAAEL | 401-436 | gb\|AAF71608.1\| |
| | AEYTAKIALLEEARR | | |
| 18 | LERQAVDQIKSQEQLAAELAE | 403-432 | gb\|ABO71780.1\|; GENE ID: |
| | YTAKIALLE | | 4064567 mag; GENE ID: 4061053 |
| | | | emm12; gb\|AAO92603.1\|; |
| | | | gb\|AAZ30332.1\|; |
| | | | dbj\|BAD14986.1\|; |
| | | | sp\|P19401\|M12_STRPY; |
| | | | gb\|AAA88573.1\| |
| 19 | LAAELAEYTAKIALLEEARRRK | 417-443 | GENE ID: 4064172 |
| | EDEVE | | MGAS10270_Spy1673 |
| 20 | EYTAKIALLEEARRRKED | 423-440 | GENE ID: 4065868 |
| | | | MGAS2096_Spy0923; GENE ID: |
| | | | 901243 SPy_1125 |
| 21 | RKPDTIEVQQMKAQAREEKH | 295-319 | GENE ID: 900682 SPy_0433 |
| | QKQLE | | |
| 22 | EYTAKIALLEEARRRKED | 423-440 | GENE ID: 901243 SPy_1125 |
| 23 | TTMDAELEF | 10-18 | GENE ID: 3572590 exoA; GENE |
| | | | ID: 900670 exoA |
| 24 | DRMAALRAKEELERQ | 392-406 | GENE ID: 901375 pyk |
| 25 | RAKEELERQAVDQ | 398-410 | GENE ID: 901105 SPy_0956 |
| 26 | SAELSSEGIRDDRNEEKR | 500-517 | GENE ID: 901583 asnA |
| 27 | KTKEELH | 458-464 | GENE ID: 900805 lysS |
| 28 | NIYEKDDKLTPKIGFPWS | 226-243 | GENE ID: 3572553 |
| | | | M5005_Spy_0341 |
| 29 | KPINVRVTTMDAELE | 3-17 | GENE ID: 3571605 valS; GENE ID: |
| | | | 901817 valS |
| 30 | NIYEKDDKLTPKIGFPWS | 226-243 | GENE ID: 900672 prtS |
| 31 | LRAKEELERQAVDQIKSQEQL | 397-421 | GENE ID: 3572544 ftsK; GENE ID: |
| | AAEL | | 900700 SPy_0458 |
| 32 | SQEQLAAELAE | 413-423 | GENE ID: 3572521 |
| | | | M5005_Spy_0388; GENE ID: |
| | | | 900713 SPy_0473 |
| 33 | QAREEKHQKQLER | 308-320 | sp\|P50470\|SPH_STRP1 |
| 34 | KEELERQAVDQIKSQEQLAAE | 400-463 | GENE ID: 900756 smc |
| | LAEYTAKIALLEEARRRKEDEV | | |
| | EEWQHRAKEAQDDLVKTKEEL | | |
| 35 | WQHRAKEAQDDLVKTKEE | 445-462 | GENE ID: 901867 SPy_2197 |
| 36 | RAKEELERQAVDQ | 398-410 | GENE ID: 3571713 |
| | | | M5005_Spy_1206 |
| 37 | QLAAELAE | 416-423 | GENE ID: 3571562 |
| | | | M5005_Spy_1331 |
| 38 | QEQLAAELAE | 414-423 | GENE ID: 900783 SPy_0567 |
| 39 | RAKEELERQAVD | 398-409 | GENE ID: 901529 SPy_1479 |
| 40 | MAALRAKEEL | 394-403 | GENE ID: 900393 abiR |
| 41 | IAQDLEMYGIN | 194-204 | GENE ID: 901292 SPy_1180 |
| 42 | QEVRKENPL | 68-76 | GENE ID: 901418 dnaN |
| 43 | ETAVLLGSYAVQAKFGDYNKE | 120-140 | GENE ID: 900605 SPy_0319 |
| 44 | EAQDDLVKTKE | 451-461 | GENE ID: 3571880 |
| | | | M5005_Spy_1022 |
| 45 | KLFFLQ | 100-105 | GENE ID: 900521 SPy_0201 |
| 46 | RRKEDEVEEWQHRAKE | 436-451 | GENE ID: 901160 acoA |
| 47 | IALLEEARRRKEDEVE | 428-443 | GENE ID: 902060 SPy_1844 |
| 48 | KGFPTWL | 53-59 | GENE ID: 900745 thrS |
| 49 | QLFDQVVKTIGLRE | 28-41 | GENE ID: 902087 glnA |
| 50 | EELIQDIT | 91-98 | GENE ID: 3571899 |
| | | | M5005_Spy_1002 |
| 51 | DQIKSQEQLAAELAEYTAK | 409-427 | GENE ID: 3572183 fbp; GENE ID: |
| | | | 901151 fbp |
| 52 | DKYKTLR | 563-569 | GENE ID: 3571138 rgg |
| 53 | WYFGLH | 43-48 | GENE ID: 3571820 |
| | | | M5005_Spy_1079 |
| 54 | VLLGSYAVQAKFGD | 123-136 | GENE ID: 3571504 |
| | | | M5005_Spy_1389; GENE ID: |
| | | | 901938 SPy_1695 |
| 55 | EKDDKLTPKIGFPW | 229-242 | GENE ID: 901619 SPy_1939 |
| 56 | WLKLDKKVSAQ | 58-68 | GENE ID: 900858 SPy_0664 |
| 57 | DNAMLEYLKIAQDLE | 185-199 | GENE ID: 3572002 citC |
| 58 | IALLEEARRRKEDEVEEWQHR | 428-463 | GENE ID: 901352 SPy_1252 |
| | AKEAQDDLVKTKEEL | | |
| 59 | DNAMLEYLKIAQDLE | 185-199 | GENE ID: 901301 citC |
| 60 | KEAQDDL | 450-456 | GENE ID: 901592 hsdM |
| 61 | EDVAEELIQ | 87-95 | GENE ID: 901918 proA |
| 62 | KLDKKVSAQEVRKENPLQF | 60-78 | GENE ID: 901621 cysS |
| 63 | DKYKTLR | 563-569 | GENE ID: 901694 ropB |
| 64 | EGILSDEIY | 108-116 | GENE ID: 901252 xpt |
| 65 | IAQDLEMYGIN | 194-204 | GENE ID: 901376 pfkA |
| 66 | GTDLWLG | 213-219 | GENE ID: 3571226 polC |
| 67 | RTHNDII | 547-553 | GENE ID: 3572302 rexA; GENE ID: |
| | | | 900953 rexA |
| 68 | AEELIQDITQKLFFLQVKEGILS | 90-116 | GENE ID: 900845 aspC |
| | DEIY | | |
| 69 | ERELSEQ | 362-368 | GENE ID: 901849 SPy_1602 |
| 70 | PIDKKAPDF | 259-267 | GENE ID: 900939 atpD |
| 71 | ILSDEIY | 100-116 | GENE ID: 900529 SPy_0216 |
| 72 | AQEVRKENPLQFKFRAKFYPE | 67-94 | GENE ID: 901641 uppS |
| | DVAEELI | | |
| 73 | KVSAQEVRK | 64-72 | GENE ID: 901147 speH |
| 74 | AELAEY | 419-424 | GENE ID: 901957 infB |
| 75 | GTDLWLG | 213-219 | GENE ID: 901636 polC |
| 76 | TKKAEREL | 358-365 | GENE ID: 901715 sagP |
| 77 | PEDVAEELIQDI | 86-97 | GENE ID: 901831 gidA |
| 78 | VSYHVQE | 481-487 | GENE ID: 3572110 |
| | | | M5005_Spy_0784; GENE ID: |
| | | | 901189 SPy_1061 |
| 79 | PNTTGKQLFD | 22-31 | GENE ID: 901775 SPy_2136 |
| 80 | QRVMDQ | 155-160 | GENE ID: 901731 SPy_2091 |
| 81 | HNENMRQ | 554-560 | GENE ID: 901317 fhs.1 |
| 82 | KVSAQEVRKENP | 64-75 | GENE ID: 901309 guaA |
| 83 | DVAEELIQDIT | 88-98 | GENE ID: 900617 SPy_0338 |

A further autoantigenic protein in use according to the present invention is Serpin B5. The autoantigenic protein Serpin B5 belongs to the Serpin family, more particularly to the ovserpin subfamily. It is secreted in normal mammary epithelial cells and occurs in the extracellular space. Serpin B5 is known as a tumor suppressor, which blocks the growth, invasion, and metastatic properties of mammary tumors. The expression of Serpin B5 is upregulated in lesional plaque psoriasis. As it does not undergo the s (stressed) to r (relaxed) conformational transition characteristic of active serpins, it exhibits no serine protease inhibitory activity. Serpin B5 is also known as Maspin or protease inhibitor 5. In the context of the present invention, the autoantigenic protein Serpin B5 has preferably a sequence as deposited under UniProtKB/Swiss-Prot entry P36952, GI number GI 142377273 (gene name SERPINB5, Synonym P15 from Homo sapiens (TaxID: 9606)). More preferably, the autoantigenic protein Serpin B5 comprises a sequence according to SEQ ID NO: 2 or a sequence showing an identity of at least about 60%, preferably of at least about 70% or about 80%, even more preferably of at least about 90% or about 95%, and most preferably an identity of at least about 99% with a sequence according to SEQ ID NO: 2. In another preferred embodiment the autoantigen is a variant or fragment of the above defined protein Serpin B5.

Alternatively or additionally, the autoantigenic protein Serpin B5 as defined herein may be provided in the form of at least one epitope of Serpin B5. Preferably, an epitope of Serpin B5 comprises a sequence having a length of about 5 to 35 contiguous amino acids or more, more preferably a length of about 5 to 20 contiguous amino acids, and most preferably a length of about 5 to 15 contiguous amino acids of the sequence according to SEQ ID NO: 2. In a preferred embodiment the autoantigen comprises at least one epitope of Serpin B5 that has a common sequence with a GAS epitope.

According to a specifically preferred embodiment, epitopes from Serpin B5 are selected from the following sequences according to SEQ ID NOs: 84-144:

| | | aa position on | |
|---|---|---|---|
| SEQ ID NO: | aa sequence of epitope | SEQ ID NO: 2: | homologies to specific gene |
| 84 | HFENVKDIPFGFQTVTSD | 59-76 | Ref\|ZP_00366590.1\| |
| 85 | KSLNLSTEFISSTKRPYAKELE | 96-147 | GENE ID: 900852 SPy_0657 |
| | TVDFKDKLEETKGQINNSIKDL | | |
| | TDGHFENI | | |
| 86 | AKELETVDFKDKLE | 113-126 | GENE ID: 900432 adcR; GENE |
| | | | ID: 994164 adcR; |
| | | | ref\|ZP_00365496.1\| |
| 87 | ETVDFKDKLEETKGQINNSIKD | 117-138 | gb\|ABD72239.1\|; GENE ID: |
| | | | 4064149 spyCEP; |
| | | | gb\|ABD72249.1\|; |
| | | | gbA\|BD72254.1\|; |
| | | | gb\|ABD72247.1\|; GENE ID: |
| | | | 3572553 M5005_Spy_0341; |
| | | | GENE ID: 3573281 prtS; GENE |
| | | | ID: 900672 prtS; |
| 88 | VDFKDKLEETKGQINNSI | 119-136 | GENE ID: 4963612 SpyM51305; |
| | | | GENE ID: 994938 spyM18_0733 |
| 89 | FKDKLEETKGQINNSIKDLTDG | 121-161 | GENE ID: 3572749 |
| | HFENILADNSVNDQTKILV | | M5005_Spy_0165 |
| 90 | DKLEETKGQINNSIK | 123-137 | GENE ID: 900826 regR |
| 91 | DKLEETKGQINNSIKDL | 123-139 | GENE ID: 2942369 M6_Spy1173 |
| | | | (an drei Stellen) |
| 92 | LEETKGQINNSIKDL | 125-139 | GENE ID: 4963445 SpyM51048; |
| | | | GENE ID: 2942140 |
| | | | M6_Spy0042; GENE ID: |
| | | | 1009750 SpyM3_1435; |
| 93 | DQTKILVVNAA | 155-165 | GENE ID: 4963222 recR; GENE |
| | | | ID: 4066535 recR; GENE ID: |
| | | | 2941577 recR; GENE ID: |
| | | | 901482 recR; GENE ID: |
| | | | 1065919 SPs0779; |
| 94 | MMNMEATF | 197-204 | GENE ID: 4962784 purB; GENE |
| | | | ID: 4067339 purB; GENE ID: |
| | | | 4063301 purB; GENE ID: |
| | | | 4062091 purB; GENE ID: |
| | | | 2942100 M6_Spy0082; GENE |
| | | | ID: 3573102 purB; GENE ID: |
| | | | 900394 purB; GENE ID: 993642 |
| | | | purB; GENE ID: 1008344 purB; |
| | | | ref\|ZP_00366589.1\| |
| 95 | KIIELPFQNKHLSMFILLPKDVE | 215-253 | GENE ID: 4963518 SpyM51157; |
| | DESTGLEKIEKQLNSE | | GENE ID: 4067838 |
| | | | MGAS10750_Spy0742; GENE |
| | | | ID: 4064016 |
| | | | MGAS10270_Spy0710; GENE |
| | | | ID: 4062254 |
| | | | MGAS9429_Spy0706; GENE ID: |
| | | | 2941648 M6_Spy0670; GENE |
| | | | ID: 3574803 M28_Spy0632; |
| | | | GENE ID: 1008883 |
| | | | SpyM3_0569; GENE ID: 994068 |
| | | | spyM18_0903; GENE ID: |
| | | | 1066283 SPs1285; |
| | | | ref\|ZP_00366186.1\|; |
| 96 | KIIELPFQNKHLSMFILLPKDVE | 215-265 | GENE ID: 901008 SPy_0843 |
| | DESTGLEKIEKQLNSESQWTN | | |
| | PSTMA | | |
| 97 | KDVEDESTGLEKIEKQLNSES | 234-254 | gb\|AAB92602.1\| |
| 98 | KDVEDESTGLEKIEKQLNSES | 234-264 | GENE ID: 2942369 M6_Spy1173 |
| | LSQWTNPSTM | | |
| 99 | ENLGLKHIFSEDTSD | 289-303 | GENE ID: 4964401 SpyM50022; |
| | | | GENE ID: 4063212 |
| | | | MGAS10270_Spy0023; GENE |
| | | | ID: 4067230 |
| | | | MGAS10750_Spy0023; GENE |
| | | | ID: 4060495 |
| | | | MGAS9429_Spy0023; GENE ID: |
| | | | 2940827 M6_Spy0072; GENE |
| | | | ID: 3573092 purL; GENE ID: |
| | | | 995242 purL; GENE ID: 900385 |
| | | | SPy_0025; GENE ID: 1008334 |
| | | | purL; GENE ID: 1066336 |
| | | | SPs0021; ref\|ZP_00366599.1\|; |
| 100 | KVCLEITEDG | 321-330 | GENE ID: 4964345 SpyM51009; |
| | | | GENE ID: 4063581 |
| | | | MGAS10270_Spy0868; GENE |
| | | | ID: 4062341; |
| | | | MGAS9429_Spy0864; GENE ID: |
| | | | 2941263; M6_Spy0775, GENE |
| | | | ID: 3573497 M28_Spy0729; |
| | | | GENE ID: 993596 spyM18_1008; |
| | | | GENE ID: 901159 SPy_1025; |
| | | | GENE ID: 1008974 |
| | | | SpyM3_0660; |
| 101 | ELNADHPFIYI | 347-357 | GENE ID: 4067288 yaaA; GENE |
| | | | ID: 4062925 yaaA; GENE ID: |
| | | | 4061369 yaaA; GENE ID: |
| | | | 3574502 yaaA; GENE ID: |
| | | | 901741 SPy_2104; GENE ID: |
| | | | 1010105 SpyM3_1790; GENE |
| | | | ID: 994207 spyM18_2163; |
| 102 | IRHNKTRNIIF | 358-368 | GENE ID: 4063972 |
| | | | MGAS10270_Spy0112 |
| 103 | KIIELPFQNKHLSMFILLPKDVE | 215-265 | GENE ID: 901008 SPy_0843 |
| | DESTGLEKIEKQLNSESLSQW | | |
| | TNPSTMA | | |
| 104 | DKLEETKGQINNSIK | 123-137 | GENE ID: 900826 regR |
| 105 | MMNMEATF | 197-204 | GENE ID: 900394 purB |
| 106 | FKDKLEETKGQINNSIKDLTDG | 121-161 | GENE ID: 3572749 |
| | HFENILADNSVNDQTKILV | | M5005_Spy_0165 |
| 107 | KSLNLSTEFISSTKRPYAKELE | 96-147 | GENE ID: 900852 SPy_0657 |
| | TVDFKDKLEETKGQINNSIKDL | | |
| | TDGHFENI | | |
| 108 | ENLGLKHIFSEDTSD | 289-303 | GENE ID: 900385 SPy_0025 |
| 109 | AKELETVDFKDKLE | 113-126 | GENE ID: 900432 adcR |
| 110 | KVCLEITEDG | 321-330 | GENE ID: 901159 SPy_1025 |
| 111 | DQTKILVVNAA | 155-165 | GENE ID: 901482 recR |
| 112 | ETVDFKDKLEETKGQINNSIKD | 117-138 | GENE ID: 3572553 |
| | | | M5005_Spy_0341; GENE ID: |
| | | | 900672 prtS |
| 113 | ELNADHPFIYI | 347-357 | GENE ID: 901741 SPy_2104 |
| 114 | ELNADHPFIYII | 347-358 | GENE ID: 900604 SPy_0317 |
| 115 | LPFQNKHLS | 219-227 | GENE ID: 3572110 |
| | | | M5005_Spy_0784; GENE ID: |
| | | | 901189 SPy_1061 |
| 116 | ETKGQINNSIKDLTDG | 127-142 | GENE ID: 3571262 salB; GENE |
| | | | ID: 901598 salB |
| 117 | EPLGNVLFS | 23-31 | GENE ID: 3571262 salB; GENE |
| | | | ID: 901598 salB |
| 118 | HFENVKDIPF | 59-68 | GENE ID: 901792 hisS |
| 119 | MDALQLANS | 1-9 | GENE ID: 3571529 tkt; GENE ID: |
| | | | 901924 tkt |
| 120 | FILLPKDVEDE | 229-239 | GENE ID: 3571502 |
| | | | M5005_Spy_1387; GENE ID: |
| | | | 901936 SPy_1693 |
| 121 | ILVVNAAYFVGKWMKKFPESE | 159-179 | GENE ID: 901667 dppC |
| 122 | DLFKQL | 14-19 | GENE ID: 901001 carB |
| 123 | WTNPSTMA | 258-265 | GENE ID: 3571591 hyl; GENE |
| | | | ID: 901847 SPy_1600 |
| 124 | KLEETKGQINNSIKDL | 124-139 | GENE ID: 3571438 |
| | | | M5005_Spy_1440 |
| 125 | ALQLANSAF | 3-11 | GENE ID: 3572764 slo; GENE |
| | | | ID: 900490 slo |
| 126 | NSAFAVDLFKQ | 8-18 | GENE ID: 900664 SPy_0401 |
| 127 | LGNVLFSPICLSTSLSL | 25-41 | GENE ID: 900786 SPy_0570 |
| 128 | MSETKGVALSNVIHKVCLEITE | 307-329 | GENE ID: 901161 acoB; GENE |
| | D | | ID: 3572156 acoB |
| 129 | ENILAD | 145-150 | GENE ID: 900745 thrS |
| 130 | KFKVEKMIDPK | 275-285 | GENE ID: 3572544 ftsK; GENE |
| | | | ID: 900700 SPy_0458 |
| 131 | VPGARILQHKDELNADHPFIYII | 336-364 | GENE ID: 3572334 pheT; GENE |
| | RHNKTR | | ID: 900946 pheT |
| 132 | RNIIFFGKF | 364-372 | GENE ID: 3572458 |
| | | | M5005_Spy_0442; GENE ID: |
| | | | 900759 SPy_0535 |
| 133 | DALQLANSA | 2-10 | GENE ID: 3572915 purK; GENE |
| | | | ID: 900392 purK |
| 134 | DFKDKLE | 120-126 | GENE ID: 901408 SPy_1324 |
| 135 | GQINNSIK | 130-137 | GENE ID: 900546 SPy_0244 |
| 136 | FIYIIRH | 354-360 | GENE ID: 901062 SPy_0908 |
| 137 | LSMFILL | 226-232 | GENE ID: 3571922 glmS |
| 138 | EIGQVLHFENVKDIPF | 53-68 | GENE ID: 3572483 drrA; GENE |
| | | | ID: 900746 SPy_0518 |
| 139 | GDSIEV | 331-336 | GENE ID: 901637 proS |
| 140 | VDKSLNLS | 94-101 | GENE ID: 900706 SPy_0464 |
| 141 | FENVKDIPFGF | 60-70 | GENE ID: 901302 SPy_1193 |
| 142 | LEKIEKQLNS | 243-252 | GENE ID: 900558 SPy_0258 |
| 143 | ILWNAA | 159-165 | GENE ID: 901181 lepA |
| 144 | LSMFILL | 226-232 | GENE ID: 901373 glmS |

A further autoantigen of the present invention is Peroxiredoxin-2. The autoantigenic protein Peroxiredoxin-2 is a homodimeric protein, which occurs in the cell and is involved in redox regulation of the cell. It is synonymously known as EC 1.11.1.15, Thioredoxin peroxidase 1, Thioredoxin-dependent peroxide reductase 1, Thiol-specific antioxidant protein TSA or as PRP. Peroxiredoxin-2 reduces peroxides with reducing equivalents provided through the thioredoxin system. It is not able to receive electrons from glutaredoxin but may play an important role in eliminating peroxides generated during metabolism. Peroxiredoxin-2 might participate in the signaling cascades of growth factors and tumor necrosis factor-alpha by regulating the intracellular concentrations of H₂O₂. In the context of the present invention, the autoantigenic protein Peroxiredoxin-2 has preferably a sequence as deposited under UniProtKB/Swiss-Prot entry P32119, GI number GI 440307 (gene name PRDX2, Synonym TDPX1 from Homo sapiens (TaxID: 9606)). More preferably, the autoantigenic protein Peroxiredoxin-2 comprises a sequence according to SEQ ID NO: 3 or a sequence showing an identity of at least about 60%, preferably of at least about 70% or about 80%, even more preferably of at least about 90% or about 95%, and most preferably an identity of at least about 99% with a sequence according to SEQ ID NO: 3. In another preferred embodiment the autoantigen is a variant or fragment of the above defined protein Peroxiredoxin-2.

Alternatively or additionally, the autoantigenic protein Peroxiredoxin-2 as defined herein may be provided in the form of at least one epitope of Peroxiredoxin-2. Preferably, an epitope of Peroxiredoxin-2 comprises a sequence having a length of about 5 to 35 contiguous amino acids or more, more preferably a length of about 5 to 20 contiguous amino acids, and most preferably a length of about 5 to 15 contiguous amino acids of the sequence according to SEQ ID NO: 3. In a preferred embodiment the autoantigen comprises at least one epitope of Peroxiredoxin-2 that has a common sequence with a GAS epitope.

According to a specifically preferred embodiment, epitopes from Peroxiredoxin-2 are selected from the following sequences according to SEQ ID NOs: 145-203:

| | | aa position on | |
|---|---|---|---|
| SEQ ID NO: | aa sequence of epitope | SEQ ID NO: 3: | homologies to specific gene |
| 145 | IGKPAPDFKATAVVDGAFKEV | 8-188 | GENE ID: 4067175 ahpC; GENE |
| | KLSDYKGKYVVLFFYPLDFTF | | ID: 901720 ahpC; GENE ID: |
| | VCPTEIIAFSNRAEDFRKLGCE | | 2941376 M6_Spy1765; |
| | VLGVSVDSQFTHLAWINTPRK | | |
| | EGGLGPLNIPLLADVTRRLSE | | |
| | DYGVLKTDEGIAYRGLFIIDGK | | |
| | GVLRQITVNDLPVGRSVDEAL | | |
| | RLVQAFQYTDEHGEVCPAGW | | |
| | KPGSDTIKPNVD | | |
| 146 | AFKEVKLSDYKGKYVVLF | 24-41 | GENE ID: 4963950 recF; GENE ID: |
| | | | 4066600 recF; GENE ID: 4060795 |
| | | | recF; GENE ID: 3574606 recF; |
| | | | GENE ID: 1010170 recF; GENE ID: |
| | | | 2940929 recF; GENE ID: 901893 |
| | | | recF; GENE ID: 993688 recF; |
| | | | ref\|ZP_00365998.1\|; |
| 147 | EVKLSDYK | 27-34 | GENE ID: 4064180 tig; GENE ID: |
| | | | 4064792 ropA; GENE ID: 4062114 |
| | | | tig; GENE ID: 2940909 tig; GENE |
| | | | ID: 1009949 tig; GENE ID: 994326 |
| | | | tig; GENE ID: 902100 tig; |
| | | | ref\|ZP_00365717.1\|; |
| 148 | EVKLSDYKGKYVV | 27-39 | GENE ID: 4068395 fhuD; GENE ID: |
| | | | 4060779 fhuD; GENE ID: 2942550 |
| | | | M6_Spy0349; GENE ID: 900654 |
| | | | fhuD; GENE ID: 993927 fhuD; |
| | | | GENE ID: 1008596 fhuD.1; |
| | | | ref\|ZP_00365818.1\|; |
| 149 | EVKLSDYKGKYVVL | 27-40 | GENE ID: 2941135 M6_Spy0325; |
| | | | GENE ID: 3573240 M28_Spy0288; |
| | | | GENE ID: 994529 spyM18_0406; |
| | | | GENE ID: 900630 SPy_0356; |
| | | | GENE ID: 1008572 SpyM3_0258; |
| | | | ref\|ZP_00366304.1\| |
| 150 | YVVLFFYPLDFT | 37-48 | GENE ID: 4963237 SpyM50729; |
| | | | GENE ID: 4063327 |
| | | | MGAS10270_Spy1200; GENE ID: |
| | | | 4068329 MGAS10750_Spy1237; |
| | | | GENE ID: 2942151 M6_Spy1105; |
| | | | GENE ID: 3573832 M28_Spy1124; |
| | | | GENE ID: 993503 spyM18_1395; |
| | | | GENE ID: 901454 SPy_1385; |
| | | | GENE ID: 1009371 SpyM3_1056; |
| | | | ref\|ZP_00366004.1\| |
| 151 | LFFYPLDFT | 40-48 | GENE ID: 4061269 |
| | | | MGAS9429_Spy1777 |
| 152 | FSNRAEDFRK | 58-67 | GENE ID: 4067385 |
| | | | MGAS10750_Spy1584; GENE ID: |
| | | | 4060959 MGAS9429_Spy1528; |
| | | | GENE ID: 2941322 M6_Spy1518; |
| | | | GENE ID: 3574236 M28_Spy1515; |
| | | | GENE ID: 1009872 cycD; GENE |
| | | | ID: 902021 SPy_1791; GENE ID: |
| | | | 993886 spyM18_1863; GENE ID: |
| | | | 1066329 SPs0310 |
| 153 | LNIPLLA | 99-105 | GENE ID: 4963476 SpyM51084 |
| 154 | VTRRLSEDYGVLKTDEGI | 107-124 | GENE ID: 2942602 M6_Spy1149 |
| 155 | RRLSEDYGVLKTDEGI | 109-124 | gb\|AAA99594.1\|; gb\|AAC06230.1\| |
| 156 | LKTDEGIAYRGLFIIDGKGVLR | 118-163 | GENE ID: 4067258 |
| | QITVNDLPVGRSVDEALRLVQ | | MGAS10750_Spy1114; GENE ID: |
| | AFQ | | 4063634 MGAS10270_Spy1078; |
| | | | GENE ID: 2941492 M6_Spy0953; |
| | | | GENE ID: 1009204 SpyM3_0889; |
| | | | GENE ID: 901353 SPy_1253 |
| 157 | GIAYRGLFIIDGKGVLR | 123-139 | GENE ID: 4963535 SpyM51191; |
| | | | GENE ID: 3573429 ebsA; GENE |
| | | | ID: 900973 SPy_0800; GENE ID: |
| | | | 1008848 SpyM3_0534; |
| | | | ref\|ZP_00366453.1\| |
| 158 | IIDGKGVLRQI | 131-141 | GENE ID: 3572438 |
| | | | M5005_Spy_0461 |
| | | | GENE ID: 900777 SPy_0558 |
| | | | GENE ID: 994804 spyM18_0622 |
| | | | ref\|ZP_00366233.1\| |
| 159 | VGRSVDEALRLV | 148-159 | GENE ID: 4062523 |
| | | | MGAS10270_Spy0812; GENE ID: |
| | | | 4060964 MGAS9429_Spy0553; |
| | | | GENE ID: 994312 spyM18_1287; |
| | | | GENE ID: 1009653 SpyM3_1338; |
| | | | GENE ID: 1009270 SpyM3_0955 |
| 160 | EALRLVQ | 154-160 | GENE ID: 4066911 |
| | | | MGAS10750_Spy0892; GENE ID: |
| | | | 4064147 MGAS10270_Spy0856; |
| | | | GENE ID: 2941579 M6_Spy0765; |
| | | | GENE ID: 3573487 M28_Spy0719; |
| | | | GENE ID: 901150 SPy_1012; |
| | | | GENE ID: 994371 spyM18_0996; |
| | | | GENE ID: 1008965 SpyM3_0651; |
| | | | ref\|ZP_00365765.1\| |
| 161 | NVDDSKEYFSK | 186-196 | GENE ID: 4067256 |
| | | | MGAS10750_Spy0116 |
| 162 | KEYFSKHN | 191-198 | GENE ID: 1008415 SpyM3_0101 |
| | | | GENE ID: 1065717 SPs0103 |
| 163 | IGKPAPDFKATAVVDGAFKEV | 8-188 | GENE ID: 901720 ahpC |
| | KLSDYKGKYVVLFFYPLDFTF | | |
| | VCPTEIIAFSNRAEDFRKLGCE | | |
| | VLGVSVDSQFTHLAWINTPRK | | |
| | EGGLGPLNIPLLADVTRRLSE | | |
| | DYGVLKTDEGIAYRGLFIIDGK | | |
| | GVLRQITVNDLPVGRSVDEAL | | |
| | RLVQAFQYTDEHGEVCPAGW | | |
| | KPGSDTIKPNVD | | |
| 164 | AFKEVKLSDYKGKYVVLF | 24-41 | GENE ID: 901893 recF |
| 165 | EVKLSDYKGKYVVL | 27-40 | GENE ID: 900630 SPy_0356 |
| 166 | EVKLSDYK | 27-34 | GENE ID: 902100 tig |
| 167 | EVKLSDYK | 27-34 | GENE ID: 3571271 tig |
| 168 | EALRLVQ | 154-160 | GENE ID: 901150 SPy_1012 |
| 169 | GIAYRGLFIIDGKGVLR | 123-139 | GENE ID: 900973 SPy_0800 |
| 170 | EVKLSDYKGKYVV | 27-39 | GENE ID: 900654 fhuD |
| 171 | FSNRAEDFRK | 58-67 | GENE ID: 902021 SPy—1791; |
| | | | GENE ID: 3571370 |
| | | | M5005_Spy_1525 |
| 172 | LKTDEGIAYRGLFIIDGKGVLR | 118-163 | GENE ID: 901353 SPy_1253 |
| | QITVNDLPVGRSVDEALRLVQ | | |
| | AFQ | | |
| 173 | IIDGKGVLRQI | 131-141 | GENE ID: 3572438 |
| | | | M5005_Spy_0461; GENE ID: |
| | | | 900777 SPy_0558 |
| 174 | YVVLFFYPLDFT | 37-48 | GENE ID: 901454 SPy_1385 |
| 175 | RQITVNDLPVG | 139-149 | GENE ID: 3571305 pepXP |
| 176 | RQITVNDLPV | 139-148 | GENE ID: 901737 SPy_2097 |
| 177 | RQITVNDLPVG | 139-149 | GENE ID: 902070 pepXP |
| 178 | GLFIID | 128-133 | GENE ID: 901760 mutL |
| 179 | LLADVTRRLSED | 103-114 | GENE ID: 901469 sodA |
| 180 | VDEALR | 152-157 | GENE ID: 901414 obgE |
| 181 | DEALRLV | 153-159 | GENE ID: 3572006 |
| | | | M5005_Spy_0914 |
| 182 | ITVNDLPVG | 141-149 | GENE ID: 900448 SPy_0108 |
| 183 | FKEVKLSDY | 25-33 | GENE ID: 900814 pepF |
| 184 | KGVLRQ | 135-140 | GENE ID: 900864 SPy_0671 |
| 185 | DEALRLV | 153-159 | GENE ID: 901304 SPy_1198 |
| 186 | IIAFSNR | 55-61 | GENE ID: 3572306 dnaG |
| 187 | YPLDF | 43-47 | GENE ID: 3572301 rexB |
| 188 | YRGLFIID | 126-133 | GENE ID: 3571605 valS |
| 189 | DTIKPNVD | 181-188 | GENE ID: 3571700 |
| | | | M5005_Spy_1193; GENE ID: |
| | | | 901516 SPy_1464 |
| 190 | IIAFSNR | 55-61 | GENE ID: 900957 dnaG |
| 191 | VLFFY | 39-43 | GENE ID: 900925 SPy_0743 |
| 192 | YPLDF | 43-47 | GENE ID: 900952 rexB |
| 193 | YRGLFIID | 126-133 | GENE ID: 901817 valS |
| 194 | DGAFKEV | 22-28 | GENE ID: 901560 divlVAS |
| 195 | YFSKHN | 193-198 | GENE ID: 3572290 |
| | | | M5005_Spy_0622 |
| 196 | SVDEALRL | 151-158 | GENE ID: 3571356 uvrA |
| 197 | NRAEDF | 60-65 | GENE ID: 900852 SPy_0657 |
| 198 | RKEGGLGPLNIP | 91-102 | GENE ID: 900677 nrdE.1 |
| 199 | YFSKHN | 193-198 | GENE ID: 900980 SPy_0807 |
| 200 | EVLGVSVD | 71-78 | GENE ID: 3571681 |
| | | | M5005_Spy_1213 |
| 201 | EVLGVSVDSQFTHL | 71-84 | GENE ID: 901312 SPy_1208 |
| 202 | SVDEALRL | 151-158 | GENE ID: 902044 uvrA |
| 203 | DTIKPNVD | 181-188 | GENE ID: 901429 map |

The fourth autoantigen that can be used according to the present invention is Heat shock protein beta-1 (HspB1). The Heat shock protein beta-1 (HspB1) is synonymously known as Heat shock 27 kDa protein, HSP27, Stress-responsive protein 27, SRP27, Estrogen-regulated 24 kDa protein or 28 kDa heat shock protein. It is involved in stress resistance and actin organization. It's human homolog may suppress polyglutamine-mediated cell death. HspB1 is located in the cytoplasm and the nucleus and is cytoplasmic in interphase cells. It colocalizes with mitotic spindles in mitotic cells and translocates to the nucleus during heat shock. It is furthermore expressed in response to environmental stresses such as heat shock, or estrogen stimulation in MCF-7 cells. The expression of HspB1 is upregulated in lesional plaque psoriasis. Mutations in the human gene of HspB1 are associated with various neuropathies and some forms of Charcot-Marie-Tooth disease [RGD] (Charcot-Marie-Tooth disease type 2F (CMT2F)). CMT2F is a form of Charcot-Marie-Tooth disease, the most common inherited disorder of the peripheral nervous system. In the context of the present invention, the autoantigenic protein HSPB1 has preferably a sequence as deposited under UniProtKB/Swiss-Prot entry PO4792, GI number GI 32477 (gene name HSPB1, Synonym HSP27 from Homo sapiens (TaxID: 9606)). More preferably, the autoantigenic protein HSPB1 comprises a sequence according to SEQ ID NO: 4 or a sequence showing an identity of at least about 60%, preferably of at least about 70% or about 80%, even more preferably of at least about 90% or about 95%, and most preferably an identity of at least about 99% with a sequence according to SEQ ID NO: 4. In another preferred embodiment the autoantigen is a variant or fragment of the above defined protein HSPB1.

Alternatively or additionally, the autoantigenic protein HSPB1 as defined herein may be provided in the form of at least one epitope of HSPB1. Preferably, an epitope of HSPB1 comprises a sequence having a length of about 5 to 35 contiguous amino acids or more, more preferably a length of about 5 to 20 contiguous amino acids, and most preferably a length of about 5 to 15 contiguous amino acids of the sequence according to SEQ ID NO: 4. In a preferred embodiment the autoantigen comprises at least one epitope of HSPB1 that has a common sequence with a GAS epitope.

According to a specifically preferred embodiment, epitopes from Heat shock protein beta-1 (HspB1) are selected from the following sequences according to SEQ ID NOs: 204-232:

| | | aa position on | |
|---|---|---|---|
| SEQ ID NO: | aa sequence of epitope | SEQ ID NO: 4: | homologies to specific gene |
| 204 | PEEWSQW | 39-45 | gb\|AAF64990.1\|AF232526_1; |
| | | | gb\|AAF65018.1\|AF232554_1; |
| | | | gb\|AAF64808.1\|AF232344_1; |
| | | | gbA\|AF64774.1\|AF232310_1; |
| 205 | RVSLDVNHFAPDELTVNHFAP | 96-114 | gb\|ABD72242.1\|; |
| | DELTVKTK | | GENE ID: 4062222 spyCEP; GENE |
| | | | ID: 4064149 spyCEP; |
| | | | gb\|ABD72249.1\|; gb\|ABD72244.1\|; |
| | | | gb\|ABD72241.1\|; gb\|ABD72243.1\|; |
| | | | gb\|ABD72246.1\|; |
| | | | gb\|ABD72248.1\|; gb\|ABD72253.1\|; |
| | | | gb\|ABD72247.1\|; GENE ID: |
| | | | 2942311 M6_Spy0367; |
| | | | gb\|ABA33824.1\|; GENE ID: |
| | | | 3572553 M5005_Spy_0341; GENE |
| | | | ID: 3573281 prtS; GENE ID: |
| | | | 995061 spyM18_0464; GENE ID: |
| | | | 1008612 prtS; GENE ID: 1065192 |
| | | | SPs1559; |
| 206 | RVSLDVNHFAPDELTVNHFAP | 96-118 | GENE ID: 900672 prtS |
| | DELTVKTKDGVV | | |
| 207 | RVSLDVNHFAPDELT | 96-110 | GENE ID: 4066747 |
| | | | MGAS10750_Spy0339; |
| | | | gb\|ABD72254.1\|; |
| | | | ref\|ZP_00365806.1\|; |
| 208 | VNHFAPDELTVKTK | 101-114 | GENE ID: 4963758 cspA; GENE ID: |
| | | | 4066748 spyCEP |
| 209 | VNHFAPDELTVKTKDGVVEI | 101-120 | gb\|ABD72239.1\| |
| 210 | VKTKDGVVEITGKHEERQDEH | 111-134 | GENE ID: 4063141 |
| | GYI | | MGAS10270_Spy1532; GENE ID: |
| | | | 2941311 M6_Spy1459; GENE ID: |
| | | | 901959 SPy_1723; |
| 211 | TKDGVVEIT | 113-121 | GENE ID: 4063622 |
| | | | MGAS10270_Spy1451 |
| 212 | EAAKSDETAAK | 195-205 | sp\|Q1J532\|NUSB_STRPF; |
| | | | sp\|Q1JF82\|NUSB_STRPD; GENE |
| | | | ID: 4067366 nusB; GENE ID: |
| | | | 4062627 nusB; GENE ID: 2941640 |
| | | | nusB; GENE ID: 3571351 nusB; |
| | | | GENE ID: 3574253 nusB; |
| | | | sp\|Q5XA94\|NUSB_STRP6; GENE |
| | | | ID: 993895 nusB; GENE ID: |
| | | | 1065484 nusB; |
| 213 | VNHFAPDELTVKTKDGVV | 101-118 | GENE ID: 900672 prtS |
| 214 | RVSLDVNHFAPDELT | 96-110 | GENE ID: 900672 prtS; GENE ID: |
| | | | 3572553 M5005_Spy_0341 |
| 215 | EAAKSDETAAK | 195-205 | GENE ID: 3571351 nusB |
| 216 | VNHFAPDELTVKTK | 101-114 | GENE ID: 3572553 |
| | | | M5005_Spy_0341 |
| 217 | VKTKDGVVEITGKHEERQDEH | 111-134 | GENE ID: 901959 SPy_1723 |
| | GYI | | |
| 218 | RLPEEWSQWL | 37-46 | GENE ID: 3571618 lacZ; GENE ID: |
| | | | 901834 SPy_1586 |
| 219 | LATQSNEITIPVTF | 172-185 | GENE ID: 3572379 agaD; GENE |
| | | | ID: 900828 agaD |
| 220 | YSRALS | 73-78 | GENE ID: 3571108 pepO; GENE |
| | | | ID: 901735 pepO |
| 221 | LDVNHF | 99-104 | GENE ID: 3571262 salB; GENE ID: |
| | | | 901598 salB |
| 222 | LSRQLSSGVSEIRHT | 77-91 | GENE ID: 901717 clpC |
| 223 | EEWSQW | 40-45 | GENE ID: 900864 SPy_0671 |
| 224 | DEHGYI | 129-134 | GENE ID: 900658 upp |
| 225 | DELTVKTKDG | 107-116 | GENE ID: 901549 xseA |
| 226 | AKSDETAAK | 197-205 | GENE ID: 901569 murD |
| 227 | SDETAAK | 199-205 | GENE ID: 902039 nusB |
| 228 | LTVKTKDGV | 109-117 | GENE ID: 900638 xerD |
| 229 | DVNHFA | 100-105 | GENE ID: 3572334 pheT; GENE |
| | | | ID: 900946 pheT |
| 230 | DVNHF | 100-104 | GENE ID: 900375 SPy_0012 |
| 231 | TKDGVVEIT | 113-121 | GENE ID: 901877 SPy_1625 |
| 232 | VKTKDGVVEITGKHEER | 111-127 | GENE ID: 901429 map |

The present invention also covers the use of fragments of the above defined autoantigenic proteins or peptides. In the context of the present invention “fragments” of autoantigenic proteins or peptides as defined herein may typically comprise those sequences in which the sequence of the encoded antigen is N- and/or C-terminally and/or intrasequentially truncated. Preferably, such fragments show an identity of at least about 60%, preferably of at least about 70% or about 80%, even more preferably of at least about 90% or about 95%, and most preferably an identity of at least about 99% with a sequence of an antigenic protein as defined herein or a corresponding part thereof. Such fragments may also be obtained from the above defined epitopes.

According to a further embodiment, variants of the above defined autoantigenic proteins or peptides can be used, which includes variants of the above defined autoantigenic proteins or peptides as defined herein, which includes variants of the full-length autoantigenic proteins or peptides as defined herein as well as of their fragments or epitopes as defined above. In the context of the present invention those encoded amino acid sequences, i.e. the above defined autoantigenic proteins or peptides as well as their epitopes or fragments as defined above, and their encoding nucleic acid sequences, in particular fall under the term “variants”, which comprise (a) conservative amino acid substitution(s) compared to their physiological sequences. Substitutions in which amino acids which originate from the same class are exchanged for one another are called conservative substitutions. In particular, these are amino acids having aliphatic side chains, positively or negatively charged side chains, aromatic groups in the side chains or amino acids, the side chains of which can enter into hydrogen bridges, e.g. side chains which have a hydroxyl function. This means that e.g. an amino acid having a polar side chain is replaced by another amino acid having a likewise polar side chain, or, for example, an amino acid characterized by a hydrophobic side chain is substituted by another amino acid having a likewise hydrophobic side chain (e.g. serine (threonine) by threonine (serine) or leucine (isoleucine) by isoleucine (leucine)). Insertions and substitutions are possible, preferably at those sequence positions, which cause no modification to the three-dimensional structure or do not affect the binding region. Modifications to a three-dimensional structure by insertion(s) or deletion(s) can easily be determined e.g. using CD spectra (circular dichroism spectra) (Urry, 1985, Absorption, Circular Dichroism and ORD of Polypeptides, in: Modern Physical Methods in Biochemistry, Neuberger et al. (ed.), Elsevier, Amsterdam). Preferably, variants as defined above, show an identity of at least about 60%, preferably of at least about 70% or about 80%, even more preferably of at least about 90% or about 95%, and most preferably an identity of at least about 99% with a sequence of an antigenic protein as defined herein, or, if a fragment or an epitope is used, with the sequence of said fragment or epitope, respectively. The same, of course, analogously may be applied to antibodies, as defined below. When variants of the above defined proteins are provided, more preferably variants of fragments or epitopes as defined above, such variants may lead to analogue peptides that can modify the immunogenic peptide ligand for the T-cell receptor. Thus, such variants are regarded as analogues derived from the original antigenic proteins, fragments or epitopes. They may carry amino acid substitutions at T-cell receptor contact residues, wherein T-cell receptor engagement by these variants may alter or impair normal T cell function. Variants as defined above may therefore act as antagonists that may specifically modulate or inhibit T cell activation induced by the wild-type antigenic peptide as defined above. Moreover, variants as defined above may also act as antagonists that may specifically modulate or inhibit a humoral immune response induced by the wild-type antigenic peptide as defined above. Treatment with such variants may selectively suppress pathogenic T cells or a humoral immune response, and, thereby, suppress the autoimmune response in autoimmune disorders.

In order to determine the percentage to which two sequences (amino acid sequences, preferably the autoantigenic protein or peptide sequences as defined above, their fragments, variants or epitopes, or the nucleic acid sequences encoding those sequences, e.g. DNA or RNA sequences) are identical, the sequences can be aligned in order to be subsequently compared to one another. Therefore, e.g. gaps can be inserted into the sequence of the first sequence and the component at the corresponding position of the second sequence can be compared. If a position in the first sequence is occupied by the same component as is the case at a position in the second sequence, the two sequences are identical at this position. The percentage to which two sequences are identical is a function of the number of identical positions divided by the total number of positions. The percentage to which two sequences are identical can be determined using a mathematical algorithm. A preferred, but not limiting, example of a mathematical algorithm which can be used is the algorithm of Karlin et al. (1993), PNAS USA, 90:5873-5877 or Altschul et al. (1997), Nucleic Acids Res., 25:3389-3402. Such an algorithm is integrated in the BLAST program or, alternatively, for nucleic acid sequences, in the NBLAST program. Sequences which are identical to the sequences of the present invention to a certain extent can be identified by this program.

An autoantigen of the present invention, or a fragment, variant or epitope thereof, as defined above, may be furthermore labelled to allow detection of said autoantigenic protein in a qualitative and/or quantitative determination. Such a label may comprise any label known in the art, e.g., without being limited thereto, “markers”, for example radioactive markers such as radioactive isotopes, fluorescence markers, including fluorescence groups, chemoluminescent groups, metal colloids, coupled enzymes, etc., more preferably a label selected from the following group:
(i) radioactive labels, i.e. radioactive phosphorylation or a radioactive label selected from radioactive isotopes of sulphur, phosphor, selenium, cobalt, iron, hydrogen, carbon, nitrogen, iod, etc., preferably selected from ³H, ¹²⁵I, ¹³¹I, ³²P, ⁵⁷Co, ⁷⁵Se, ⁵⁹Fe, ¹⁴C and ³⁵S, etc.;
(ii) fluorescent groups, wherein the fluorescent group may be selected from any fluorescent protein or peptide, e.g. from a group comprising fluorescein, the blue fluorescent protein (BFP), the green fluorescent protein (GFP), the photo activatable-GFP (PA-GFP), the yellow shifted green fluorescent protein (Yellow GFP), the yellow fluorescent protein (YFP), the enhanced yellow fluorescent protein (EYFP), the cyan fluorescent protein (CFP), the enhanced cyan fluorescent protein (ECFP), the monomeric red fluorescent protein (mRFP1), the kindling fluorescent protein (KFP1), aequorin, the autofluorescent proteins (AFPs), or the fluorescent proteins JRed, TurboGFP, PhiYFP and PhiYFP-m, tHc-Red (HcRed-Tandem), PS-CFP2 and KFP-Red (all available from EVRΩGEN, see also www.evrogen.com), or Alexa 350, Alexa 430, AMCA, BODIPY 630/650, BODIPY 650/665, BODIPY-FL, BODIPY-R6G, BODIPY-TMR, BODIPY-TRX, carboxyfluorescein, Cascade Blue, Cy3, Cy5, 6-FAM, Fluorescein, HEX, 6-JOE, Oregon Green 488, Oregon Green 500, Oregon Green 514, Pacific Blue, REG, Rhodamine Green, Rhodamine Red, ROX, TAMRA, TET, Tetramethylrhodamine, or Texas Red, or other suitable fluorescent proteins, peptides or molecule, e.g. fluoresceine isothiocyanate (FITC), phycoerythrin (PE), allophycocyanine (APC), etc., or fragments or variants thereof;
(iii) chemoluminescent groups, e.g. for time-resolved chemoluminescence, including lanthanoid complexes;
(iv) metal colloids (e.g. gold, silver, etc.) as particles;
(v) enzymes such as horseradish peroxidase, alkaline phosphatase, beta-galactosidase, or any other suitable enzymes.

In one embodiment an autoantigen of the present invention as defined above can be in immobilized form, and, therefore, may furthermore carry a group for immobilization on a solid phase, or a fragment, variant or epitope thereof, as defined above, which allows binding of the autoantigenic protein to a sample carrier, support, or matrix. In the context of the present invention, such groups for immobilization on a solid phase are or comprise, without being limited thereto, peptide sequences, such as a His₈-tag, a streptavidine tag (Strep-tag) (particularly preferably in combination with a biotin molecule), or glutathione-S-transferase-tag (GST-Tag), biotin (particularly preferably in combination with a streptavidine tag (Strep-tag)), etc.; Alternatively, such groups for immobilization on a solid phase comprise a moiety, which may be already present in the autoantigenic protein or may be added chemically and which allows binding of the autoantigenic protein to a sample carrier, support, or matrix. Such moieties may be, e.g., moieties selected from the group, consisting of, without being limited thereto, a hydroxyl moiety, an amino moiety, a carboxyl moiety, an alkoxy moiety, a chlormethyl moiety, an aldehyde moiety, a hydrazide moiety, etc. Such moieties allow e.g. a chemical coupling to a sample carrier, support, or matrix, e.g. by formation of peptide bonds, amid bonds, isourea (derivate) bonds, hydrazone bonds, etc. According to a further alternative, such groups for immobilization on a solid phase likewise may comprise a linker, which allows binding of the autoantigenic protein to a sample carrier, support, or matrix. In the context of the present invention, such a linker may be any anorganic, organic or bioorganic molecule that is suitable to bind the autoantigenic protein to a sample carrier, support, or matrix. Preferably, such a linker may have at least two and optionally 3, 4, or more reactive groups, which allow binding of the linker to a sample carrier, support, or matrix as well as to the autoantigenic protein. More preferably, such reactive groups of a linker are selected from the group, consisting of, without being limited thereto, a hydroxyl moiety, an amino moiety, a carboxyl moiety, an alkoxy moiety, a chlormethyl moiety, an aldehyde moiety, a hydrazide moiety, etc. Preferably, such a linker or the above groups for immobilization make use of the terminal amino or carboxyl moiety of the autoantigenic protein or of other moieties of the autoantigenic protein, e.g. hydroxyl moieties, amino moieties, thiol-moieties, or an alkoxy moiety, etc., e.g., by forming van der Waals-bonds, or by forming covalent bonds such as e.g. sulphur-sulphur bonds, peptide bonds, amide bonds, isourea derivate bonds, etc. Other suitably linkers, may be selected, without being limited thereto, from the group consisting of glycol, glycerol and glycerol derivatives, 2-aminobutyl-1,3-propanediol and 2-aminobutyl-1,3-propanediol derivatives/scaffold, pyrrolidine linkers or pyrrolidine-containing organic molecules, etc. Glycerol or glycerol derivatives or a 2-aminobutyl-1,3-propanediol derivative/scaffold. Alternatively, the above autoantigenic proteins or a fragment, variant or epitope thereof, may be bound to a sample carrier, support, or matrix by use of van der Waals-bonds or other non covalent interactions. Furthermore, in the context of the present invention, a suitable sample carrier, support, or matrix may any membrane suitable for the present case and known to a skilled person, e.g. blotting membranes such as a nitrocellulose membrane or a polyvinylidendiflouride membrane (PVDF membrane), etc.

Moreover, instead of using the autoantigenic protein or peptide, a nucleic acid encoding the protein or peptide can also be used. In the context of the present invention, a nucleic acid may be selected from any biological or synthetic source or may be contained in nucleic acid libraries or databases, e.g. databases for genomic DNA, artificial chromosomes, mini chromosomes, subgenomic DNA, cDNA, synthetic DNA sequences, RNA sequences, e.g. mRNAs, or may directly be derived from such sequences or combinations thereof. In this context, a messenger RNA (mRNA) is typically an RNA, which is composed of (at least) several structural elements, e.g. an optional 5′-UTR region, an upstream positioned ribosomal binding site followed by a coding region, an optional 3′-UTR region, which may be followed by a poly-A tail (and/or a poly-C-tail). In the context of the present invention, nucleic acids furthermore may be selected from circular or linear and/or single-, double stranded or partially double stranded nucleic acids, e.g. genomic DNA, subgenomic DNA, cDNA, synthetic DNA sequences, or RNA sequences such as mRNAs, and may encode any of the autoantigenic proteins or peptides as defined herein, or their fragments, variants or epitopes, as well as antibodies as defined below. Preferably, the reading frame of such a nucleic acid is not interrupted by a stop codon. If the nucleic acid sequence is an RNA, the RNA may be, without being limited thereto, a coding RNA, a circular or linear RNA, a single- or a double-stranded RNA (which may also be regarded as an RNA due to non-covalent association of two single-stranded RNA) or a partially double-stranded RNA (which is typically formed by a longer and a shorter single-stranded RNA molecule or by two single stranded RNA-molecules, which are about equal in length, wherein one single-stranded RNA molecule is in part complementary to the other single-stranded RNA molecule and both thus form a double-stranded RNA in this region).

Nucleic acids as defined herein may be part of a suitable nucleic acid sequence. In the context of the present invention a suitable nucleic acid sequence includes, e.g., a DNA element, that provides autonomously replicating extrachromosomal plasmids derived from animal viruses (e.g. bovine papilloma virus, polyomavirus, adenovirus, or SV40, etc.). Such suitable nucleic acids are known to a skilled person and may be reviewed e.g. in “Cloning Vectors” (Eds. Pouwels P. H. et al. Elsevier, Amsterdam-New York-Oxford, 1985, ISBN 0 444 904018). Suitable nucleic acids are also intended to include any suitable nucleic acid sequence known to a skilled person, such as plasmids, phages, viruses such as SV40, CMV, Baculo virus, Adeno virus, Sindbis virus, transposons, IS-elements, phasmids, phagemides, cosmides, linear or circular DNA or RNA. Linear DNA is typically used for integration in mammalian cells. Preferably, the type of nucleic acid sequences used in the context of the present invention corresponds to the specific host cell requirements. Suitable commercially available nucleic acids include pSPORT, pBluescriptllSK, pBIIKS, pPIC9, pGEX, pMAL, pFLAG, pCR2.1, the baculovirus expression vector pBlueBac, and the prokaryotic expression vector pcDNAII, all of which may be obtained from Invitrogen Corp., San Diego, Calif.

Autoantigenic proteins and peptides of the present invention as well as their variants, fragments or epitopes (and antibodies as defined below), and their encoding nucleic acids may be obtained from any synthetic or naturally occurring source, which is available to a skilled person. E.g., autoantigenic proteins and peptides as defined herein, their variants, fragments or epitopes (and antibodies as defined below) may be derived from a protein or peptide library or may be transcribed from a nucleic acid library, such as a cDNA library, or may be obtained from any living or dead tissue, from a sample obtained from e.g. a human, animal or bacterial source. E.g., nucleic acids encoding autoantigenic proteins and peptides, their variants, fragments or epitopes (and antibodies as defined below) may be directly derived from any nucleic acid library, such as a cDNA library, or may also be obtained from any living or dead tissue, from a sample obtained from e.g. a human, animal or bacterial source. A “sample” in the sense of this invention is typically to be understood as any type of solution, solid or tissue to be tested, in particular solutions of medically relevant substances, such as e.g. a body liquid, such as blood, lymph, serum, urine, liquor, cells, tissue, faeces or biopsies in general, also in a processed form, prepared for the sample handling or in unprocessed from, e.g. cytosolic preparations from human cells. Alternatively, autoantigenic proteins, their variants, fragments or epitopes (and antibodies as defined below) or nucleic acids encoding same may be synthetically be prepared by methods known to a person skilled in the art. As an example, autoantigenic proteins as well as their variants, fragments or epitopes (or antibodies as defined below) may be synthesized, without being limited thereto, e.g. by peptide synthesis methods as known to a skilled person, such as liquid phase peptide synthesis or solid phase peptide synthesis (SPPS) according to Merrifield, e.g. Boc SPPS, Fmoc SPPS or BOP SPPS, etc. As another example, nucleic acid sequences, which may encode the above autoantigenic proteins, their variants, fragments or epitopes, may be synthesized, without being limited thereto, e.g. by solid phase synthesis or any other suitable method for preparing nucleic acid sequences. Furthermore, substitutions, additions or eliminations of bases in these nucleic acid sequences are preferably carried out using a DNA matrix for preparation of the nucleic acid sequence or by techniques of the well known site directed mutagenesis or with an oligonucleotide ligation strategy (see e.g. Maniatis et al., Molecular Cloning: A Laboratory Manual, Cold Spring Harbor Laboratory Press, 3rd ed., Cold Spring Harbor, N.Y., 2001).

According to a further embodiment, the present invention provides antibodies directed against at least one of the autoantigenic proteins or peptides as defined above for use as a diagnostic tool or a therapeutical agent. Antibodies specifically binding with one of the autoantigens of the present invention, for example selected from Ezrin, Serpin B5, Peroxiredoxin-2 and Heat shock protein beta-1, a variant, fragment and/or an epitope of one of the proteins or peptides can be used alone or in combination.

According to the present application, the term “antibody” comprises monoclonal antibodies, polyclonal antibodies, particularly polyclonal monospecific antibodies (i.e. antibodies with different variable regions, which however all recognize a specific epitope), as well as chimeric antibodies, (anti-)anti-idiotypic antibodies (directed to the inventive antibodies, preferably directed against an antibody, which is in turn directed against at least one of the autoantigenic proteins Ezrin, Serpin B5, Peroxiredoxin-2 and Heat shock protein beta-1 (herein also termed “autoreactive antibody”), or a fragment, variant or epitope thereof, as defined above.). The term “antibody” herein furthermore comprises genetically manipulated antibodies. All of the afore mentioned antibodies may be present in bound or soluble form and may be—if appropriate—labeled as defined above, e.g. by “markers” (for example fluorescence marker, radioactive isotopes, gold marker, coupled enzymes, etc.), and/or may carry a peptide, group or linker for immobilization on a solid phase, preferably as described above for autoantigenic proteins. The term “antibody” in the meaning of the present invention typically refers to full-length antibodies of the afore mentioned antibodies. A “full-length” (monoclonal) antibody in the meaning of the present application may be any of the above mentioned inventive antibodies in its full-length form. A full-length antibody of the present invention typically comprises both the domains of the heavy chain and the light chain. The heavy chain of the inventive antibody typically includes domains C_{H}1, C_{H}2 or C_{H}3 of the constant region and the variable heavy (V_{H}) immunoglobulin domain. The light chain of the inventive antibody typically includes the variable light immunoglobulin domain (V_{L}) and the constant light immunoglobulin domain (C_{L}). Antibodies, not containing all the aforementioned domains or regions of an antibody are fragments of antibodies within the meaning of the present invention. Fragments of antibodies according to the present invention are further defined below and also encompassed by the above embodiment of the present invention. Antibodies according to the present invention may pertain to one of the following immunoglobulin classes: IgG, IgM, IgE, IgA, GILD and, if applicable, a subclass of the aforementioned classes, such as the subclasses of the IgG or their mixtures. IgG and its subclasses such as IgG1, IgG2, IgG2a, IgG2b, IgG3 or IgGM are preferred. The IgG subtypes IgG1/k or IgG2b/k are specifically preferred. Antibodies in the sense of this invention are furthermore proteins, peptides or possibly other structures produced by vertebrates or by artificial production methods, that bind with high affinity to a determined surface conformation (epitope), e.g. of one of the autoantigenic proteins Ezrin, Serpin B5, Peroxiredoxin-2 and Heat shock protein beta-1, or a fragment, variant or epitope thereof, as defined above, or of an antibody specifically binding to these autoantigenic proteins. Typically, such antibodies contain at least the variable part of immunoglobulins, and, as the case may be, at least one domain of the constant domain of immunoglobulins, too.

“Polyclonal antibodies” according to the present invention and directed against at least one of the autoantigenic proteins Ezrin, Serpin B5, Peroxiredoxin-2 and Heat shock protein beta-1 (“autoreactive antibodies”), or a fragment, variant or epitope thereof, as defined above in the meaning of the present application are typically heterogeneous mixtures of antibody molecules, produced from animal serums, that had been immunized with at least one of the autoantigenic proteins Ezrin, Serpin B5, Peroxiredoxin-2 and Heat shock protein beta-1, or a fragment, variant or epitope thereof, as defined above.

A “monoclonal antibody” according to the present invention and directed against one of the autoantigenic proteins Ezrin, Serpin B5, Peroxiredoxin-2 and Heat shock protein beta-1 (“autoreactive antibodies”), or a fragment, variant or epitope thereof, as defined above contains a fundamentally homogeneous population of antibodies, that are directed specifically to one of the autoantigenic proteins Ezrin, Serpin B5, peroxiredoxin-2 and heat shock protein beta-1, or a fragment, variant or epitope thereof, as defined above. The different antibody variants with mono-specificity may belong to the immunoglobulin classes described above. They may also be mixtures of different major primary classes or subclasses, preferentially, they consist of a homogenous mixture of IgG-antibodies. This homogeneity may also be achieved by an additional purification step (immuno-precipitation, chromatography, for example by using antibodies directed to IgG). Monoclonal antibodies may also be obtained by using methods known in the state-of-the-art (e.g. Köhler and Milstein, Nature, 256, 495-397, (1975); U.S. Pat. No. 4,376,110; Ausubel et al., Harlow and Lane “Antikörper: Laboratory Manual, Cold Spring, Harbor Laboratory (1988); Ausubel et al., (eds), 1998, Current Protocols in Molecular Biology, John Wiley & Sons, New York)). The aforementioned references are included herein in their entirety. “Monoclonal” is typically intended to mean the product of an artificial construct, in which an antibody-producing cell (B-cell) is fused with an immortalized cancer cell (hybridom), creating a hybridoma cell. Specific antibodies, that are all exclusively directed to one of the autoantigenic proteins Ezrin, serpin B5, peroxiredoxin-2 and heat shock protein beta-1, or a fragment, variant or epitope thereof, as defined above, are produced by this cell. A hybridoma-cell clone, producing monoclonal antibodies according to the present invention, is cultured in vitro.

“Genetically manipulated antibodies” may also be provided according to the present invention, which are directed to one of the autoantigenic proteins Ezrin, serpin B5, peroxiredoxin-2 and heat shock protein beta-1, or a fragment, variant or epitope thereof, as defined above. In the context of the present invention, “genetically manipulated antibodies” may be based on polyclonal or monoclonal antibodies as defined above, which have been genetically modified. Such “genetically manipulated antibodies” may be produced using methods known to a skilled person, e.g. as described in the aforementioned publications.

As a further alternative “chimeric antibodies” may be provided according to the present invention, which are directed to one of the autoantigenic proteins Ezrin, Serpin B5, Peroxiredoxin-2 and Heat shock protein beta-1, or a fragment, variant or epitope thereof, as defined above. In this context “Chimeric antibodies” are molecules, that contain different constituents, which are derived from different animal species (e.g. antibodies, showing a variable region, that is derived from a monoclonal mouse antibody and a constant region of a human immunoglobulin). Chimeric antibodies are preferably used on the one hand for the reduction of immunogenicity, if administered, and on the other hand for the increase of yield, e.g., murine monoclonal antibodies yield higher rates of production from hybridoma cell lines, however, they are also associated with a higher immunogenicity in humans. Therefore, human/murine chimeric antibodies are preferably used. Chimeric antibodies and methods for their production are known state-of-the-art methods. (Cabilly et al., Proc. Natl. Sci. USA 81: 3273-3277 (1984); Morrison et al. Proc. Natl. Acad. Sci USA 81:6851-6855 (1984); Boulianne et al. Nature 312 643-646 (1984); Cabilly et al., EP-A-125023; Neuberger et al., Nature 314: 268-270 (1985); Taniguchi et al., EP-A-171496; Morrion et al., EP-A-173494; Neuberger et al., WO 86/01533; Kudo et al., EP-A-184187; Sahagan et al., J. Immunol. 137: 1066-1074 (1986); Robinson et al., WO 87/02671; Liu et al., Proc. Natl. Acad. Sci USA 84:3439-3443 (1987); Sun et al., Proc. Natl. Acad. Sci USA 84:214218 (1987); Better et al., Science 240: 1041-1043 (1988) und Harlow und Lane, Antikarper: A Laboratory Manual, as quoted above). These references are also included in the present invention, as if disclosed in their entirety.

An “anti-idiotypic antibody” according to the present invention may also be provided according to the present invention. Such an “anti-idiotypic antibody” is typically a (monoclonal or polyclonal) antibody that recognizes a determinant, which is generally associated with the binding site of an antibody according to the present invention, i.e. an antibody directed to one of the autoantigenic proteins Ezrin, serpin B5, peroxiredoxin-2 and heat shock protein beta-1, or a fragment, variant or epitope thereof, as defined above (“autoreactive antibody”). An anti-idiotypic antibody can be produced through immunization of an animal of the same species and the same genetic type (e.g. a mice strain) as a point of origin for a monoclonal antibody (an “autoreactive antibody” as defined above), against which an anti-idiotypic antibody according to this invention is targeted. The immunized animal will then recognize the idiotypic determinants of the immunizing antibody (the “autoreactive antibody”) through the production of an antibody, that is directed to the idiotypic determinants (namely an anti-idiotypic antibody according to the present invention) (U.S. Pat. No. 4,699,880). An anti-idiotypic antibody according to the present invention may also be used as an immunogen, in order to provoke an immune response in another animal and to induce the production of a so-called anti-anti-idiotypic antibody there. The anti-anti-idiotypic antibody may be, but does not have to be, identical to the original monoclonal antibody with reference to the design of its epitope, that had caused the anti-idiotypic reaction. This allows the identification of other clones, that express antibodies of identical specificity, with the use of an antibody directed to idiotypic determinants of a monoclonal antibody. In order to induce production of anti-idiotypic antibodies in the respective animals, such as e.g. the BALB/c mouse, monoclonal antibodies, directed to one of the autoantigenic proteins Ezrin, serpin B5, peroxiredoxin-2 and heat shock protein beta-1 (“autoreactive antibodies”), or a fragment, variant or epitope thereof, as defined above, solubilized or suspended in body liquids, can be used. Cells taken from the spleen of such an immunized mouse can then be used to produce anti-idiotypic hybridoma-cell lines, that secrete anti-idiotypic monoclonal antibodies. Furthermore, anti-idiotypic monoclonal antibodies may also be coupled to a medium (KLH, “keyhole limpet hemocyanin”) and subsequently be used for further immunization of BALB/c-mice. The sera of these mice then contain anti-anti-idiotypic antibodies, that exhibit the binding properties of the original monoclonal antibodies and that are specific for a physiologic binding protein solubilized or suspended in body liquids. Therefore, the anti-idiotypic monoclonal antibodies have their own idiotypic epitopes or “idiotopes”, characterized by a similar structure as the structure of the epitope to be examined.

Furthermore, an antibody of the present invention may also be bispecific, that is to say, it may also recognize different epitopes of the autoantigens of the present invention, for example eptitopes of at least one autoantigenic protein Ezrin, Serpin B5, Peroxiredoxin-2 or Heat shock protein beta-1, or a fragment, variant or epitope thereof, as defined above, with its two paratopes, preferably two different epitopes of the same protein or a fragment, variant or epitope thereof, as defined above. Eventually, both paratopes may be structural different, however, they may still bind the same epitope or at least overlapping areas of these epitopes. According to a preferred embodiment, such an inventive bispecific antibody may recognize e.g. combinations of the above proteins, or a fragment, variant or epitope thereof, as defined above, selected from e.g. the combinations Ezrin and Serpin B5, Ezrin and Peroxiredoxin-2, Ezrin and Heat shock protein beta-1, Serpin B5 and Peroxiredoxin-2, Serpin B5 and Heat shock protein beta-1, or Peroxiredoxin-2 and Heat shock protein beta-1. Alternatively, such a bispecific antibody may recognize on the one hand side any specific label, linker or moiety as defined herein to allow immobilization on a solid phase, e.g. a sample carrier, support, or matrix; as defined above. On the other hand side, the same bispecific antibody may recognize at least one autoantigenic protein Ezrin, Serpin B5, Peroxiredoxin-2 or Heat shock protein beta-1, or a fragment, variant or epitope thereof, as defined above, or may recognize a specific determinant of a(n) (“autoreactive”) antibody, wherein the (“autoreactive”) antibody binds to one of the autoantigenic proteins Ezrin, Serpin B5, Peroxiredoxin-2 and Heat shock protein beta-1, or a fragment, variant or epitope thereof, as defined above. According to a further alternative, the bispecific antibody may already contain a group for immobilization to a solid phase and may recognize two of the above autoantigenic proteins, fragments, variants or epitopes thereof, preferably in the above combinations. A bispecific antibody, in this context may be also an anti-idiotypic antibody as defined herein, which may bind to the respective (“autoreactive”) antibodies recognizing the above autoantigenic proteins, fragments, variants or epitopes thereof, preferably in the above combinations.

In another embodiment the antibody used according to the present invention is humanized and can be any of the above defined antibodies in humanized form. Humanization of antibodies as known in the prior art can be performed by a large variety of standard methods. Therefore, human or humanized antibodies of antibodies as defined herein are also understood as antibodies according to the present invention.

“Fragments” of an antibody according to the present invention are also encompassed by the present invention. A “fragment of an antibody according to the present invention” typically may comprise any fragment of an inventive antibody as defined above, either fragments of a polyclonal or monoclonal antibody, etc. A fragment of an inventive antibody thus may comprise e.g. the constant regions of the heavy chain of the inventive antibody, e.g. C_{H}1, C_{H}2 or C_{H}3, the variable heavy (V_{H}) immunoglobulin domain, the variable light immunoglobulin domain (V_{L}), or the constant light immunoglobulin domain (C_{L}). The constant heavy immunoglobulin domain is typically an F_{c} fragment comprising the C_{H}3 domain and/or the C_{H}2 and/or the C_{H}1 domain. The variable light immunoglobulin domain is preferably an F_{ab} fragment comprising the V_{L} domain. Also encompassed by the present invention are all shortened or modified antibody fragments presenting one or two binding sites complementary to a sequence of an autoantigenic protein, a fragment, a variant or an epitope as defined herein. Such shortened or modified antibody fragments typically comprise antibody parts with a binding site corresponding to the antibody, composed of a light and a heavy chain, such as Fᵥ-, F_{ab}- or F(_{ab}′)₂-fragments or single-chain antibody fragments (scFᵥ). Shortened double strand fragments, such as Fᵥ-, F_{ab}- or F(_{ab}′)₂ are preferred. F_{ab} and F(_{ab}′)₂-fragments have no F_{c}-fragment, which would be present for instance in an intact antibody, therefore, they may be transported faster in the blood circulation and show comparably less non-specific tissue binding than intact antibodies. Moreover, because of the missing F_{c} part they cannot elicit an undesired rejection. In this context, it is stressed, that F_{ab} and F(_{ab}′)₂ fragments of antibodies according to the present invention can be used in an inventive method in the sense of the invention presented. Such fragments are typically produced by proteolytic cleavage, using enzymes, such as e.g. papain (for the production of F_{ab}-fragments) or pepsin (for the production of F(_{ab}′)₂, fragments), or by chemical oxidation or by genetic manipulation of the antibody genes. Fragments of the antibodies of the present invention, as defined above, are typically functionally homolog to the antibodies of the present invention. “Functionally homolog” in the meaning of the present invention means that a fragment, a variant, etc. of an antibody of the present invention preferably recognizes specifically a sequence of one of the autoantigenic proteins Ezrin, Serpin B5, Peroxiredoxin-2 and Heat shock protein beta-1, or a fragment, variant or epitope thereof, as defined above, or, in the case of an anti-idiotypic antibody, the antibody specifically recognizes an antibody binding to an autoantigenic protein, or a fragment, variant or epitope thereof. A “functional homolog” of an antibody of the present invention is also understood to include antibodies with increased or lowered affinity to one of the autoantigenic proteins Ezrin, Serpin B5, Peroxiredoxin-2 and Heat shock protein beta-1, or a fragment, variant or epitope thereof, as defined above. Such antibodies with a modulated activity may excite different biological properties. Furthermore, a person skilled in the art may select an antibody with a specific affinity as necessary in the respective case.

“Variants” of any of the above mentioned antibodies are also contemplated by the present invention. A variant of an antibody in the meaning of the present invention typically comprises a sequence, wherein at least one, two or more amino acids, preferably 1-5, 1-10, 1-15, 1-20, 1-50 or 1-100 amino acids, of the entire amino acid sequence of said antibody are altered, i.e. deleted, substituted or added with respect to the amino acid sequence of the full-length antibody of the present invention. Variants of antibodies of the present invention are preferably functionally homolog to the full-length non-altered antibodies of the present invention.

According to the present invention it is possible to either use at least one antibody or antibody fragment or variant as defined above or a combination of antibodies or antibody fragments or variants binding two different epitopes of the same autoantigen or two epitopes of different autoantigens. More preferably antibodies that are directed against different proteins as defined above, i.e. antibody species having different targets selected from any of the autoantigenic proteins Ezrin, Serpin B5, Peroxiredoxin-2 and Heat shock protein beta-1, can be combined wherein these targets may be selected independently from the autoantigenic proteins in native form or from fragments, variants or epitopes thereof.

In one embodiment of the present invention an antibody composition can be used that comprises a specific combination of antibodies directed against at least one of the above autoantigenic proteins (“autoreactive antibodies”) or a variant, a fragment or an epitope of those an autoantigenic proteins. More preferably a combination of at least two antibodies binding to autoantigens as defined above or variants, fragments or epitopes thereof are used in the following combinations:
Ezrin, or
Serpin B5, or
Peroxiredoxin-2, or
Heat shock protein beta-1, or
Ezrin and Serpin B5, or
Ezrin and Peroxiredoxin-2, or
Ezrin and Heat shock protein beta-1, or
Serpin B5 and Peroxiredoxin-2, or
Serpin B5 and Heat shock protein beta-1, or
Peroxiredoxin-2 and Heat shock protein beta-1, or
Ezrin and Serpin B5 and Peroxiredoxin-2, or
Ezrin and Serpin B5 and Heat shock protein beta-1, or
Ezrin and Peroxiredoxin-2 and Heat shock protein beta-1, or
Serpin B5 and Peroxiredoxin-2 and Heat shock protein beta-1, or
Ezrin and Serpin B5 and Peroxiredoxin-2 and Heat shock protein beta-1.

Alternatively or additionally, the at least one antibody of an inventive antibody composition as defined above may be an anti-idiotypic antibody according to the present invention, i.e. an antibody that recognizes a determinant, which is generally associated with the binding site of a(n) (“autoreactive”) antibody according to the present invention as defined above, wherein this (“autoreactive”) antibody selectively binds to one of the autoantigenic proteins Ezrin, Serpin B5, Peroxiredoxin-2 and Heat shock protein beta-1, or a fragment, variant or epitope thereof, as defined above. More preferably, the (at least one) anti-idiotypic antibody of the inventive antibody composition is directed against at least one (preferably different) (“autoreactive”) antibody, which recognizes at least one (preferably different) autoantigenic protein as defined above, i.e. each anti-idiotypic antibody species in the inventive diagnostic composition has a different (“autoreactive”) antibody target, which, in turn, recognizes at least one (preferably different) autoantigenic protein.

The autoantigen antibodies can be used as diagnostic tools or as therapeutical agents.

For example, the autoantigenic antibodies can be used to determine those variants, fragments or derivatives that bind with the highest affinity to the antibody to determine those variants or fragments that are most effective for diagnosis and therapy of GAS driven conditions.

Moreover, the autoantigen antibodies of the present invention can be used to detect GAS binding to these antibodies as an indication for a streptococcal infection that could result in secondary streptococcal disorders.

According to a further embodiment, the present invention also provides a composition useful as diagnostic tool for the detection of streptococcal driven conditionscomprising
(a) at least one autoantigen as defined above, and/or
(a′) at least one antibody as defined above, and
(b) optionally a carrier, excipient, and/or vehicle.

In a further embodiment the composition comprises different autoantigens and/or fragments carrying different epitopes of one or more of the autoantigens of the invention. This composition can be used to detect and/or determine autoreactive antibodies in a subject. Preferably the composition comprises at least one autoantigen of the present invention that has a sequence and/or at least one epitope in common with GAS.

In further embodiment the composition can comprise at least one autoantigen as defined above and at least one antibody binding therewith to be used for an immune therapy. Such combination can be used to elicit Fc receptor mediated regulatory immune reactions.

In a further embodiment a pharmaceutical composition is provided as therapeutical agent to treat a streptococcal driven condition comprising at least one autoantigen as defined above and a pharmaceutically acceptable carrier, adjuvant, and/or vehicle.

Moreover, instead of the autoantigen as defined above, the pharmaceutical composition can also comprise a nucleic acid encoding the autoantigen.

In another embodiment a composition is provided as diagnostic tool for the detection of streptococcal driven conditions comprising at least one antibody or antibody fragment that is directed against at least one of the autoantigens as defined above, particularly against a fragment, variant or epitope thereof. More preferably antibodies that are directed against different autoantigens or different epitopes of at least one autoantigen are used in the inventive composition.

Preferably, the inventive diagnostic or pharmaceutical composition may comprise (a) an inventive composition, comprising at least one of the autoantigenic proteins Ezrin, Serpin B5, Peroxiredoxin-2 and Heat shock protein beta-1, or a fragment, variant or epitope thereof, as defined above, preferably in any of the above combinations, wherein the at least one autoantigenic protein is either present in the inventive pharmaceutical composition as a peptide or protein or is encoded by a nucleic acid as defined herein. In other words, the inventive pharmaceutical composition may comprise (a) an inventive composition, comprising at least one, more preferably two, three or even four of the autoantigenic proteins Ezrin, Serpin B5, Peroxiredoxin-2 and Heat shock protein beta-1, in any of the above combinations, or peptides comprising at least 5 consecutive amino acid residues of one of the autoantigenic proteins having immunological activity, or a fragment, variant or epitope thereof, as defined above, or nucleic acid(s) encoding same.

If desirable the inventive pharmaceutical composition can comprise at least one antibody or antibody fragment that is directed against at least one, preferably against two, three or even four of the autoantigenic proteins Ezrin, Serpin B5, Peroxiredoxin-2 and Heat shock protein beta-1 (“autoreactive antibodies”), or a fragment, variant or epitope thereof, as defined above. More preferably, the antibodies are directed against different autoantigenic proteins as defined above, i.e. each antibody species in the inventive pharmaceutical composition has a different target selected from any of the autoantigenic proteins Ezrin, Serpin B5, Peroxiredoxin-2 and Heat shock protein beta-1, wherein these targets may be selected independently from the (native) autoantigenic proteins as defined above, or from fragments, variants or epitopes thereof.

The at least one antibody or antibody fragment may be alternatively an anti-idiotypic antibody according to the present invention or a fragment thereof, i.e. an antibody or a fragment thereof that recognizes a determinant, which is generally associated with the binding site of an antibody according to the present invention. In other words, the at least one antibody or antibody fragment may be an anti-idiotypic antibody or a fragment thereof which is directed against an inventive antibody selectively binding to one of the autoantigenic proteins Ezrin, Serpin B5, Peroxiredoxin-2 and Heat shock protein beta-1 (“autoreactive antibodies”), or a fragment, variant or epitope thereof, as defined above.

Furthermore, a pharmaceutical or diagnostic composition of the present invention as defined above may comprise (b) a pharmaceutically acceptable carrier, excipient, adjuvant, and/or vehicle. In the context of the present invention, a pharmaceutically acceptable carrier, excipient, adjuvant, or vehicle typically refers to a non-toxic carrier, excipient, adjuvant or vehicle that does not destroy the pharmacological or immunological activity of the autoantigen and/or antibody compound (or its encoding nucleic acids) with which it is formulated. Pharmaceutically acceptable carriers, excipients, adjuvants or vehicles are known in the art. These carriers, excipients, adjuvants or vehicles, that may be used in the inventive pharmaceutical composition, may be typically distinguished into solid or liquid, carriers, excipients, adjuvants, or vehicles. The skilled person can find the optimal carrier, excipient, adjuvant, or vehicle or a mixture thereof. These compounds can inter alia be used to adapt the viscosity of the composition. In this context, solid carriers excipients, and vehicles typically include e.g., but are not limited to, ion exchangers, alumina, aluminum stearate, lecithin, and salts, if provided in solid form, such as protamine sulfate, disodium hydrogen phosphate, potassium hydrogen phosphate, sodium chloride, zinc salts, colloidal silica, magnesium trisilicate, or polyvinyl pyrrolidone, or cellulose-based substances, sodium carboxymethylcellulose, polyacrylates, waxes, polyethylene-polyoxypropylene-block polymers, polyethylene glycol and wool fat. Liquid carriers, excipients, or vehicles, e.g. for aqueous or oleaginous suspensions, typically include, but are not limited to, e.g., ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, if provided in solubilized form, such as protamine sulfate, disodium hydrogen phosphate, potassium hydrogen phosphate, sodium chloride, zinc salts, or polyethylene glycol, or 1,3-butanediol, Ringer's solution, isotonic sodium chloride solution, sterile, fixed oils, any suitable bland fixed oil, e.g. including synthetic mono- or di-glycerides, fatty acids, such as oleic acid and its glyceride derivatives, natural pharmaceutically-acceptable oils, such as olive oil or castor oil, especially in their polyoxyethylated versions, wherein these oil solutions or suspensions may also contain a long-chain alcohol diluent or dispersant, such as carboxymethyl cellulose or similar dispersing agents, or commonly used surfactants, such as Tweens, Spans and other emulsifying agents or bioavailability enhancers.

An excipient is any component of a finished dosage form other than the claimed therapeutic ingredient or ingredients, particularly any component that is used to prepare a dosage form and to adapt the desired properties like viscosity, osmalility etc. The skilled person is well aware of a multitude of useful excipients for pharmaceutical and diagnostic compositions and useful components can be found in textbooks and databases.

In the context of the present invention, pharmaceutically acceptable adjuvants can be a component that enhances the composition but also a component that has an immune modulating effect. It may also be understood as any compound, which is suitable to support administration and delivery of the inventive pharmaceutical composition. Such an adjuvant may be selected from any adjuvant known to a skilled person and suitable for the present case. The adjuvant may be selected from the group consisting of, without being limited thereto, cationic peptides, including polypeptides including protamine, nucleoline, spermine or spermidine, cationic polysaccharides, including chitosan, TDM, MDP, muramyl dipeptide, pluronics, alum solution, aluminium hydroxide, ADJUMER™ (polyphosphazene); aluminium phosphate gel; glucans from algae; algammulin; aluminium hydroxide gel (alum); highly protein-adsorbing aluminium hydroxide gel; low viscosity aluminium hydroxide gel; AF or SPT (emulsion of squalane (5%), Tween 80 (0.2%), Pluronic L121 (1.25%), phosphate-buffered saline, pH 7.4); AVRIDINE™ (propanediamine); BAY R1005™ ((N-(2-deoxy-2-L-leucylamino-b-D-glucopyranosyl)-N-octadecyl-dodecanoyl-amide hydro-acetate); CALCITRIOL™ (1-alpha,25-dihydroxy-vitamin D3); calcium phosphate gel; CAPTM (calcium phosphate nanoparticles); cholera holotoxin, cholera-toxin-A1-protein-A-D-fragment fusion protein, sub-unit B of the cholera toxin; CRL 1005 (block copolymer P1205); cytokine-containing liposomes; DDA (dimethyldioctadecylammonium bromide); DHEA (dehydroepiandrosterone); DMPC (dimyristoylphosphatidylcholine); DMPG (dimyristoylphosphatidylglycerol); DOC/alum complex (deoxycholic acid sodium salt); Freund's complete adjuvant; Freund's incomplete adjuvant; gamma inulin; Gerbu adjuvant (mixture of: i) N-acetylglucosaminyl-(P1-4)-N-acetylmuramyl-L-alanyl-D-glutamine (GMDP), ii) dimethyldioctadecylammonium chloride (DDA), iii) zinc-L-proline salt complex (ZnPro-8); GM-CSF); GMDP (N-acetylglucosaminyl-(b1-4)-N-acetylmuramyl-L-alanyl-D-isoglutamine); imiquimod (1-(2-methypropyl)-1H-imidazo[4,5-c]quinoline-4-amine); ImmTher™ (N-acetylglucosaminyl-N-acetylmuramyl-L-Ala-D-isoGlu-L-Ala-glycerol dipalmitate); DRVs (immunoliposomes prepared from dehydration-rehydration vesicles); interferon-gamma; interleukin-1beta; interleukin-2; interleukin-7; interleukin-12; ISCOMS™; ISCOPREP 7.0.3.™; liposomes; LOXORIBINE™ (7-allyl-8-oxoguanosine); LT oral adjuvant (E. coli labile enterotoxin-protoxin); microspheres and microparticles of any composition; MF59™; (squalene-water emulsion); MONTANIDE ISA 51™ (purified incomplete Freund's adjuvant); MONTANIDE ISA 720™ (metabolisable oil adjuvant); MPL™ (3-Q-desacyl-4′-monophosphoryl lipid A); MTP-PE and MTP-PE liposomes ((N-acetyl-L-alanyl-D-isoglutaminyl-L-alanine-2-(1,2-dipalmitoyl-sn-glycero-3-(hydroxyphosphoryloxy))-ethyl-amide, monosodium salt); MURAMETIDE™ (Nac-Mur-L-Ala-D-Gln-OCH₃); MURAPALMITINE™ and D-MURAPALMITINE™ (Nac-Mur-L-Thr-D-isoGln-sn-glyceroldipalmitoyl); NAGO (neuraminidase-galactose oxidase); nanospheres or nanoparticles of any composition; NISVs (non-ionic surfactant vesicles); PLEURAN™ (β-glucan); PLGA, PGA and PLA (homo- and co-polymers of lactic acid and glycolic acid; microspheres/nanospheres); PLURONIC L121™; PMMA (polymethyl methacrylate); PODDS™ (proteinoid microspheres); polyethylene carbamate derivatives; poly-rA: poly-rU (polyadenylic acid-polyuridylic acid complex); polysorbate 80 (Tween 80); protein cochleates (Avanti Polar Lipids, Inc., Alabaster, Ala.); STIMULON™ (QS-21); Quil-A (Quil-A saponin); S-28463 (4-amino-otec-dimethyl-2-ethoxymethyl-1H-imidazo[4, 5-c]quinoline-1-ethanol); SAF-1™ (“Syntex adjuvant formulation”); Sendai proteoliposomes and Sendai-containing lipid matrices; Span-85 (sorbitan trioleate); Specol (emulsion of Marcol 52, Span 85 and Tween 85); squalene or Robane® (2,6,10,15,19,23-hexamethyltetracosan and 2,6,10,15,19,23-hexamethyl-2,6,10,14,18,22-tetracosahexane); stearyltyrosine (octadecyl-tyrosine hydrochloride); Theramid® (N-acetylglucosaminyl-N-acetylmuramyl-L-Ala-D-isoGlu-L-Ala-dipalmitoxypropylamide); Theronyl-MDP (Termurtide™ or [thr 1]-MDP; N-acetylmuramyl-L-threonyl-D-isoglutamine); Ty particles (Ty-VLPs or virus-like particles); Walter-Reed liposomes (liposomes containing lipid A adsorbed on aluminium hydroxide), and lipopeptides, including Pam3Cys, in particular aluminium salts, such as Adju-phos, Alhydrogel, Rehydragel; emulsions, including CFA, SAF, IFA, MF59, Provax, TiterMax, Montanide, Vaxfectin; copolymers, including Optivax (CRL1005), L121, Poloaxmer4010), etc.; liposomes, including Stealth, cochleates, including BIORAL; plant derived adjuvants, including QS21, Quil A, Iscomatrix, ISCOM; adjuvants suitable for costimulation including Tomatine, biopolymers, including PLG, PMM, Inulin; microbe derived adjuvants, including Romurtide, DETOX, MPL, CWS, Mannose, CpG nucleic acid sequences, CpG7909, ligands of human TLR 1-10, ligands of murine TLR 1-13, ISS-1018, IC31, Imidazoquinolines, Ampligen, Ribi529, IMOxine, IRIVs, VLPs, cholera toxin, heat-labile toxin, Pam3Cys, Flagellin, GPI anchor, LNFPIII/Lewis X, antimicrobial peptides, UC-1V150, RSV fusion protein, cdiGMP; and adjuvants suitable as antagonists including CGRP neuropeptide. In a preferred embodiment the adjuvant is selected basesd on the intended immunological action.

The inventive pharmaceutical composition may be administered orally, parenterally, by inhalation spray, topically, rectally, nasally, buccally, vaginally or via an implanted reservoir. The term parenteral as used herein includes subcutaneous, intravenous, intramuscular, intra-articular, intra-synovial, intrasternal, intrathecal, intrahepatic, intralesional and intracranial injection or infusion techniques.

Preferably, the inventive pharmaceutical composition may be administered by parenteral injection, more preferably by subcutaneous, intravenous, intramuscular, intra-articular, intra-synovial, intrasternal, intrathecal, intrahepatic, intralesional and intracranial injection or via infusion techniques. Sterile injectable forms of the inventive pharmaceutical compositions may be aqueous or oleaginous suspensions, e.g. using pharmaceutically acceptable carriers, adjuvants and or vehicles as defined above.

These aqueous or oleaginous suspensions may further be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1,3-butanediol. Among the above acceptable vehicles and solvents, that may be employed for injectable preparations, are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation of the inventive pharmaceutical composition.

The inventive pharmaceutical composition as defined above may also be administered orally in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

The inventive pharmaceutical composition may also be administered topically, especially when the target of treatment includes areas or organs readily accessible by topical application, e.g. including diseases of the skin or of any other accessible epithelial tissue. Suitable topical formulations are readily prepared for each of these areas or organs. For topical applications, the inventive pharmaceutical compositions may be formulated as a suitable ointment containing the autoantigenic proteins and/or antibodies as defined above, or the encoding nucleic acids, suspended or dissolved in one or more carriers. Carriers for topical administration include, but are not limited to, mineral oil, liquid petrolatum, white petrolatum, propylene glycol, polyoxyethylene, polyoxypropylene compound, emulsifying wax and water. Alternatively, the inventive pharmaceutical composition can be formulated in a suitable lotion or cream. In the context of the present invention, suitable carriers include, but are not limited to, mineral oil, sorbitan monostearate, polysorbate 60, cetyl esters wax, cetearyl alcohol, 2-octyldodecanol, benzyl alcohol and water.

The inventive pharmaceutical composition typically comprises a “safe and effective amount” of the at least one autoantigenic protein as defined herein (or a fragment, variant or epitope thereof) and/or of the at least one inventive antibody as defined herein, irrespective of whether the at least one autoantigenic protein and/or the at least one antibody is provided in the form of a peptide or protein or as a nucleic acid. As used herein, a “safe and effective amount” means an amount of the at least one autoantigenic protein as defined herein (or a fragment, variant or epitope thereof) and/or of the at least one inventive antibody as defined herein in the inventive pharmaceutical composition as defined above, or of a nucleic acid encoding same, that is sufficient to significantly induce a positive modification of a disease or disorder as defined herein. At the same time, however, a “safe and effective amount” is small enough to avoid serious side-effects, that is to say to permit a sensible relationship between advantage and risk. The determination of these limits typically lies within the scope of sensible medical judgment. A “safe and effective amount” of the at least one autoantigenic protein as defined herein (or a fragment, variant or epitope thereof) and/or of the at least one inventive antibody as defined herein, or of a nucleic acid encoding same, will furthermore vary in connection with the particular condition to be treated and also with the age and physical condition of the patient to be treated, the body weight, general health, sex, diet, time of administration, rate of excretion, drug combination, the activity of the employed specific autoantigenic protein and/or antibody as defined herein, the severity of the condition, the duration of the treatment, the nature of the accompanying therapy, the particular pharmaceutically acceptable carrier used, and similar factors, within the knowledge and experience of the accompanying doctor. The inventive pharmaceutical composition may be used for human and also for veterinary medical purposes, preferably for human medical purposes.

According to a specific embodiment, the inventive pharmaceutical composition may be provided as a vaccine. The term “vaccine” in the present application is used for defining a composition that has immunomodulating properties and typically is composed like the inventive pharmaceutical composition. More preferably, the inventive vaccine is suitable to desensitize or to hyposensitize the immune reaction of a patient to be treated towards one of the above mentioned autoantigenic proteins, or a fragment, variant or epitope thereof.

Preferably an inventive vaccine comprises (a) an inventive composition as defined above, containing at least one of the above mentioned autoantigenic proteins, a fragment, variant or epitope thereof, or a nucleic acid as encoding same, and (b) optionally a pharmaceutically acceptable carrier, adjuvant, and/or vehicle, as defined above.

Additional to the inventive composition, the inventive vaccine may comprise (c) a pharmaceutically acceptable carrier, adjuvant, and/or vehicle as defined above for the inventive pharmaceutical composition. In the specific context of the inventive vaccine, the choice of a pharmaceutically acceptable carrier is preferably determined by the manner in which the inventive vaccine is to be administered. The inventive vaccine can be administered, for example, systemically or locally. Routes for systemic administration in general include, for example, transdermal, oral, parenteral routes, including subcutaneous or intravenous injections and/or intranasal administration routes. Routes for local administration in general include, for example, topical administration routes but also transdermal, intramuscular or subcutaneous injection. More preferably, vaccines may be administered by an intradermal, subcutaneous, or intramuscular route. Inventive vaccines are therefore preferably formulated in liquid or solid form. The suitable amount of the inventive vaccine to be administered can be determined by routine experiments with animal models. Such models include, without implying any limitation, rabbit, sheep, mouse, rat, dog and non-human primate models. Preferred unit dose forms for injection include sterile solutions of water, physiological saline or mixtures thereof. The pH of such solutions should be adjusted to about 7.4. Suitable carriers for injection include hydrogels, devices for controlled or delayed release, polylactic acid and collagen matrices. Suitable pharmaceutically acceptable carriers for topical application include those which are suitable for use in lotions, creams, gels and the like. If the inventive vaccine is to be administered perorally, tablets, capsules and the like are the preferred unit dose form. The pharmaceutically acceptable carriers for the preparation of unit dose forms which can be used for oral administration are well known in the prior art. The choice thereof will depend on secondary considerations such as taste, costs and storability, which are not critical for the purposes of the present invention, and can be made without difficulty by a person skilled in the art.

The inventive vaccine can additionally contain one or more auxiliary substances in order to further modulate the immunogenicity of the vaccine, i.e. of the components thereof, if required. A synergistic action of the at least one autoantigenic protein of the inventive vaccine as defined above and of an auxiliary substance, which may be optionally also contained in the inventive vaccine as described above, is preferably achieved thereby. The vaccine typically comprises an adjuvant. The term “adjuvant” when used in the present application refers to compounds that have an immune modulating effect. In the context of the present invention the immune modulating effect is preferably a downregulation of an immune response against one of the autoantigens as defined above. Any immune modulating compound know in the art that is used for this purpose can be used for the vaccine of the present invention.

Further additives which may be included in the inventive vaccine are emulsifiers, such as, for example, Tween®; wetting agents, such as, for example, sodium lauryl sulfate; colouring agents; taste-imparting agents, pharmaceutical carriers; tablet-forming agents; stabilizers; antioxidants; preservatives.

The inventive vaccine can also additionally contain any further compound, which is known to be immune-modulating.

The autoantigens of the present invention and the specifically binding antibodies can be used in a method for hyposensitization of a patient having a streptococcal driven condition. A hyposensitization is used to induce tolerance against the autoantigens that are attacked by the patient. Furthermore the substances claimed in the present invention are useful both for determining the protein selected from the above group, or an immunologically reactive part thereof that creates autoantibodies and to provide substances useful for inducing tolerance.

Thus, that part that elicits autoantibodies or an autoimmune response can be found using the antibodies of the present invention. For example, autoantigens of the present invention are contacted with a sample of a patient who supposedly has a streptococcal driven condition, to detect that sequence that is most reactive with autoantibodies. Based on this knowledge, a substance or a mixture of substances selected from autoantigens or variants or derivates thereof can be used for the hyposensitization where increasing amounts of the substance or mixture of substances are administered to “adapt” the immune system to these sequences and to induce tolerance. Methods for inducing tolerance or for hyposensitization are known to a person skilled in the art. Generally, the present invention provides the means that can be used in these methods.

A hyposensitization can also be performed if the autoimmune response is a cellular immune response. Eliciting principle can be detected by determining the release of factors like cytokines in reaction to one or more of the autoantigens of the present invention. Again, as soon as the eliciting principle has been determined, the corresponding autoantigens can be used for inducing cellular immune tolerance.

According to another embodiment the present invention provides a process for the detection of a streptococcal driven condition comprising qualitatively and/or quantitatively determining antibodies against at least one autoantigen selected from Ezrin, Serpin B5, Peroxiredoxin-2, Heat shock protein beta-1, and/or at least one fragment, variant or epitope thereof, in a sample. The detection of such autoantigen antibodies provides evidence for the presence of a streptococcal driven condition.

Antibodies can be detected in a sample using common detection methods. The sample typically is a sample that has been obtained from or provided by a patient and is typically to be understood as any type of solution to be tested in the above inventive method. If the sample is obtained from a natural source, the sample typically comprises biopsies or medically relevant solutions, such as e.g. solutions of cells, tissues, body liquid(s), such as blood, lymph, serum, urine, liquor, either in unprocessed form or also in a processed form, prepared for the sample handling. Likewise, it is preferable, if the sample to be determined in the inventive method contains liquid, preferably body liquid, more preferably human body liquid, in particular blood or human blood. In this context, a “body liquid” is to be understood as any liquid obtained from the body of a vertebrate, in particular a mammal, in particular of a human being. In the case of human beings, this would for instance be blood, urine or lymph, but also (cytosolic) preparations from human cells. Such solutions may be obtained by e.g. taking blood or a cell sample, a cytosolic preparation, etc. from a patient using methods known to a skilled person, and, if necessary mixing said sample, e.g. with a liquid as defined above, or a buffer, preferably a physiological buffer as defined herein, etc. Alternatively, if the sample is obtained from a synthetic source, the sample typically comprises liquids, i.e. solutions or a buffer, preferably a physiological buffer as defined herein, etc. Moreover, synthetic solutions may be prepared or provided for calibration or comparison, such synthetic solutions typically either contain at least one of the autoantigens based on or derived from Ezrin, Serpin B5, Peroxiredoxin-2 and Heat shock protein beta-1 (in a defined concentration and combination) or antibodies against these autoantigens (in a defined concentration and combination), e.g. for calibration of the inventive method for qualitatively and/or quantitatively detecting the presence of at least one antibody against at least one of the autoantigenic proteins Ezrin, Serpin B5, Peroxiredoxin-2 and Heat shock protein beta-1.

The present invention also provides a method for detecting a streptococcal driven condition, particularly an auto-immune condition, by qualitatively and/or quantitatively determining the presence of at least one antibody against at least one of the autoantigenic proteins Ezrin, Serpin B5, Peroxiredoxin-2 and Heat shock protein beta-1 in a sample, or a fragment, variant or epitope thereof, that comprises the following steps:
(a) obtaining or providing a sample from a patient or a synthetic or natural source putatively containing at least one antibody against one or more of the autoantigenic proteins Ezrin, Serpin B5, Peroxiredoxin-2 and heat shock protein beta-1, or a fragment, variant or epitope thereof;
(b) contacting the sample with an autoantigen as defined before to allow binding of antibodies present in the sample with the at least one autoantigen and
(c) qualitatively and/or quantitatively determining the presence of the at least one antibody using biophysical or biomolecular detection methods.

According to the present invention a streptococcal driven condition can be detected by contacting a sample with at least one autoantigen as described above, that is selected from Ezrin, Serpin B5, Peroxiredoxin-2 and Heat shock protein beta-1, and/or a peptide comprising at least 5 consecutive amino acid residues of one of these autoantigenic proteins having immunological activity, or fragments, variants, or epitopes thereof and/or mixtures of at least two of the proteins and/or peptides to allow forming of complexes of the at least one autoantigen and antibodies present in the sample. Thereafter the presence and/or amount of the formed complexes is detected. The detection can be qualitatively and/or quantitatively, wherein the presence and/or amount of antibodies is evidence for a humoral immune response that has occurred in the patient. The detection of the presence of antibodies against the at least one autoantigen of the present invention in a subject is an indication that an autoimmune response has occurred. Only one autoantigen or variant, fragment or epitope can be used. More preferred at least two autoantigens or fragments, variants or epitopes thereof are used, where fragments, variants or epitopes of one autoantigen or of more than one autoantigen can be used.

In the method of the present invention the sample is added to at least one autoantigen of the present invention. Usually the sample is in the form of a fluid and the autoantigen can be added directly or, preferably, as a solution or dispersion. In another embodiment the autoantigen or a fragment, variant or epitope thereof, may be bound to a carrier, support, or matrix directly or by a linker such that the binding part is available for forming the complex with an antibody. Any method that is known to the skilled person can be used.

The sample is contacted with the autoantigen, or fragment, a variant or an epitope thereof to allow the forming of a complex.

Typically, when contacting the sample with the antigens and/or antibodies the binding occurs upon incubating the sample (putatively containing an (“autoreactive”) antibody against one of the above autoantigenic proteins) with the inventive composition, wherein incubation times and incubation temperatures are typically selected by a person skilled in the art. The term “incubation”, as used herein is to be understood as a reaction condition, in which the reaction partners, in other words the (“autoreactive”) antibody and a corresponding autoantigenic protein are allowed to react with each other. The incubation is generally carried out for a limited period of time, before the start of the qualitative and/or quantitative measurement. As an example, without being limited thereto, suitable incubation times may vary from 10 seconds minute up to 48 hours. Suitable incubation temperatures may e.g. vary, without being limited thereto, from about 0° C. to about 40° C., e.g. from about 0° C. to about 10° C., from about 10° C. to about 25° C, from about 25° C. to 40° C., preferably from 30° C. to 37° C. Suitable incubation solutions may comprise, without being limited thereto, PBS, or Na-Carbonate-buffer, or 0,1 M Na-Carbonate buffer, etc., or any other suitable incubation solution known to a skilled person, containing e.g. Tris buffered saline or Tween 20, etc. After incubating the sample (putatively containing an (“autoreactive”) antibody against one of the above autoantigenic proteins) with the inventive composition, the supernatant may be discarded and the sample carrier, support or matrix may be washed with a suitable washing solution, typically a solution containing, without being limited thereto, PBS, or PBS/0,05% Tween 20 or TBS/0.5% Tween 20, or saline/0.1% Tween 20, or any other suitable washing solution known to a skilled person, containing e.g. Tris buffered saline (with or without detergents), Tween 20, etc.

In the method of the present invention for qualitatively and/or quantitatively detecting the presence of an antibody binding to at least one of the autoantigens defined in claim 1 or 2, also referred to as (“autoreactive”) antibody in this application, in a sample, the “autoreactive” antibody against one of the above autoantigens, the protein(s) preferably bound to a sample carrier, support, or matrix, may be qualitatively and/or quantitatively detected using suitable biophysical or biomolecular detection methods. Suitable biophysical or biomolecular detection methods for qualitatively detecting the (“autoreactive”) antibody comprise any suitable method known in the art. Such methods include, without being limited thereto, methods as applied for qualitative or quantitative assays, e.g. for Enzyme-linked Immunosorbent Assay (ELISA), ELISPOT-Assay, Western-Blot or Immunoassays. Such methods comprise e.g. optical, radioactive or chromatographic methods, preferably when using any of the above labels, markers or linkers, more preferably fluorescence detection methods, radioactivity detection methods, Coomassie-Blue staining, Silver staining or other protein staining methods, electron microscopy methods, methods for staining tissue sections by immunohistochemistry or by direct or indirect immunofluorescence, etc. Such methods may be applied either with the autoreactive antibody or may involve the use of further tools, e.g. the use of a secondary antibody, specifically binding to the constant part of the autoreactive antibody. Such a secondary antibody may be any inventive anti-idiotypic antibody as defined above or any further (anti-idiotypic) antibody, suitable to specifically bind to an autoreactive antibody against one of the above autoantigens. Such a secondary antibody, either an inventive anti-idiotypic antibody as defined above or a further suitable (anti-idiotypic) antibody, may be labelled as indicated above to allow a specific detection of the secondary antibody.

In the method of the present invention for detecting the presence of at least one antibody against one of the above defined autoantigens in a sample a qualitative or a quantitative determination can be carried out. “Qualitative determination” in the context of the inventive method is to be understood as any method for specifically identifying the presence of a specific autoreactive antibody, i.e. an autoreactive antibody directed against one or more of specific proteins selected from the autoantigenic proteins Ezrin, Serpin B5, Peroxiredoxin-2 and Heat shock protein beta-1, or a fragment, variant or epitope thereof. “Quantitative determination” in the context of the inventive method is to be understood as any method for determination of an antibody or (antibody) proteins or peptides, e.g. fragments, variants or epitopes thereof, known by a skilled person suitable for quantifying the amount of a autoreactive antibody or a secondary antibody, e.g. an anti-idiotypic antibody, in a sample. This includes explicitly, e.g. quantification of an antibody by using e.g. optical, radioactive or chromatographic methods, preferably when using any of the above labels, more preferably optical, radioactive or chromatographic methods applying a concurrent standard. As an example, the inventive method may be carried out with a test sample as a concurrent standard, containing a defined amount of a, probably recombinantly produced, autoreactive antibody against at least one of the above autoantigenic proteins, and in parallel with a second sample, which is derived from a patient and contains an unknown amount of an autoreactive antibody to be determined against at least one of the above autoantigenic proteins. A comparison of the defined amount of the autoreactive antibody in the test sample with the amount of the autoreactive antibody in the second sample will allow a precise determination of the amount of autoreactive antibody in the second sample. Such a method is suitable for any of the above labels. A concurrent standard may be applied either parallel to carrying out the inventive method or, e.g., prior to said method, by preparing a standard curve, which may be used in the subsequent quantification.

In a further embodiment the present invention also provides a method for detecting the presence of a cellular immune response in a patient, optionally combined with detecting the presence of a humoral immune response. As GAS can induce humoral responses as well as cellular responses, particularly humoral and/or cellular autoimmune responses, the detection of a humoral and/or a cellular immune response in a patient can be useful. If a patient shows a condition that is supposed to be caused by a cellular autoimmune response a corresponding test can be made which comprises that a sample is provided or obtained from a patient and a cellular immune response is detected in the sample preferably by qualitatively and/or quantitatively detecting the secretion of at least one factor involved in a cellular immune response after contact with an autoantigen. An increase of the amount of the factor is evidence for a cellular autoimmune response. Both methods can be combined.

Usually the diagnosis method is an in vitro method on a sample obtained from the patient where the sample can be any of the samples as described above. It is also possible to detect a reaction in a cell or tissue in vivo with a test kit provided by the present invention.

The detection of a humoral and/or cellular response, particularly autoimmune response, is used for the diagnosis of a condition in a patient putatively suffering from a (post-streptococcal) disease induced and/or mediated by group A beta-haemolytic streptococci (GAS) as defined herein, particularly of psoriasis vulgaris (plaque psoriasis), erythrodermic psoriasis, pustular psoriasis, psoriatic arthritis, and autoimmune disorders including rheumatic fever and heart disease, post-streptococcal glomerulonephritis, or a variety of pediatric autoimmune neuropsychiatric disorders associated with streptococcal infections (PANDAS).

The method for detecting the presence of a humoral and additionally a cellular immune response in a patient comprises detecting a humoral immune response in the sample, preferably by qualitatively and/or quantitatively detecting at least one autoreactive antibody against one or more of the autoantigens as defined in claim 1, for example at least one autoantigenic protein selected from Ezrin, Serpin B5, Peroxiredoxin-2 and heat shock protein beta-1, or and/or a peptide comprising at least 5 consecutive amino acid residues of one of these autoantigenic proteins having innumological activity, or fragments, variants, or epitopes thereof and/or mixtures of at least two of the proteins and/or peptides a fragment, variant or epitope thereof, using biophysical or biomolecular detection methods. In this context, a humoral immune response (HIS) as detected in step (b) of the inventive method may be understood as a reaction that is mediated by secreted antibodies, produced in the cells of the B lymphocyte lineage (B cell). In the context of the present invention those secreted antibodies are typically autoreactive antibodies, preferably antibodies reactive with at least one of the herein defined autoantigenic proteins Ezrin, Serpin B5, Peroxiredoxin-2 and Heat shock protein beta-1, and/or a peptide comprising at least 5 consecutive amino acid residues of one of these autoantigenic proteins having immunological activity, or fragments, variants, or epitopes thereof and/or mixtures of at least two of the proteins and/or peptides. The term “Humoral immunity” was formed due to involvment of substances found in the humours, or body fluids. Particularly, humoral immunity refers to antibody production, and the accessory processes that accompany it, including: TH2 activation and cytokine production, germinal center formation and isotype switching, affinity maturation and memory cell generation. It also refers to the effector functions of antibody, which include pathogen and toxin neutralization, classical complement activation, and opsonin promotion of phagocytosisand pathogen elimination.

In the method for detecting the presence of a cellular immune response in a patient preferably at least one factor or the secretion of at least one factor involved in cellular immune response is detected qualitatively and/or quantitatively using commonly known detection methods, for example biophysical or biomolecular methods. In the context of the present invention, a cellular immune response or cell-mediated immune response, particularly cellular autoimmune resposne shall be understood as an immune response, that does not involve antibodies but rather involves the activation of macrophages, natural killer cells (NK), antigen-specific T-lymphocytes, and the release of various cytokines in response to an antigen. Particularly, a cellular immune response or cell-mediated immunity typically comprises as a first step activating antigen-specific T-lymphocytes, in the context of the present invention e.g. with at least one of the herein defined autoantigenic proteins Ezrin, Serpin B5, Peroxiredoxin-2 and Heat shock protein beta-1, or a fragment, variant or epitope thereof. Such a cellular immune response or cell-mediated immunity typically comprises in a second step activating of macrophages and natural killer cells, enabling them to destroy those intracellular pathogens or antigenic proteins; and in a third step stimulating cells to secrete a variety of cytokines that influence the function of other cells involved in adaptive immune responses and innate immune responses. Such cytokines comprises e.g. IL-1, IL-2, IL-3, IL-4, IL-5, IL-6, IL-7, IL-8, IL-9, IL-10, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, INF-alpha, IFN-beta, IFN-gamma, GM-CSF, G-CSF, M-CSF, LT-beta or TNF-alpha, growth factors, etc. For the purposes of the present invention, detection of a cellular immune response, particularly cellular autoimmune response may be carried out by using different (qualitative and/or quantitative) biophysical or biomolecular detection methods. Such methods include, inter alia, assays (suitable) for measuring changes in cell proliferation or cell metabolism or detecting T-cell mediated cytotoxicity, e.g. induced by recognition of the autoantigenic proteins, including but not restricted to assays measuring incorporation of ³H-thymidine or of Bromodeoxyuridin, activation-induced Ca²⁺ influx, release of ⁵¹chromium or lactate dehydrogenase (LDH), or other assays measuring changes in metabolic activity, cell replication, cell numbers, or cell death, measurement of phosphatidylinositol (PI) hydrolysis in activated T lymphocytes, fluorescence polarization as an early measure of T lymphocyte stimulation, measurement of lymphoproliferation at the single-cell Level by flow cytometry, digital image analysis of lymphocyte activation, detecting ubiquitinated T-cell antigen receptor subunits by immunoblotting, measurement of activation markers on the T cell surface by immunofluorescence, determination of CD45 tyrosine phosphatase activity in T lymphocytes, measurement of protein tyrosine phosphorylation in T-cell subsets by flow cytometry, biochemical analysis of activated T lymphocytes: protein phosphorylation and Ras, ERK, and JNK activation, activation of heterotrimeric GTP-Binding Proteins upon TCR/CD3 engagement, or qualitatively and/or quantitatively detecting the secretion of at least one of the above cytokines using biophysical or biomolecular detection methods. By way of example, “qualitative determination” with respect to detection of the secretion of cytokines may be understood as any method for specifically identifying the identity of at least one of these cytokines, whereas “quantitative determination” shall be understood as any method for quantifying the amount of such a cytokine in a sample. This includes explicitly, e.g. quantification of a cytokine by using e.g. optical, radioactive or chromatographic methods, preferably applying a concurrent standard. As an example, the inventive method may be carried out with a test sample as a concurrent standard, containing a defined amount of a cytokine and in parallel with a second sample, which is derived from a patient and contains an unknown amount of a cytokine secreted due to a cellular immune response. A comparison of the defined amount of the cytokine in the test sample with the amount of the cytokine in the second sample will allow a precise determination of the amount of cytokine in the second sample. A concurrent standard may be applied either parallel to carrying out the inventive method or, e.g., prior to said method, by preparing a standard curve, which may be used in the subsequent quantification.

As outlined above it is possible according to the present invention to determine the presence of a humoral auto-immune response, a cellular auto-immune response or both a humoral and cellular auto-immune response against at least one of the autoantigens of the present invention in a patient dependent from the condition of the patient. A concurrent presence of a humoral and a cellular immune response against at least one of the autoantigens of the present invention might strongly indicate the presence of a streptococcal driven disease, i.e. is an indication that an autoimmune disorder elicited by a GAS infection is present in the patient. Examples for those GAS driven autoimmune disorders are psoriasis, rheumatic fever and heart disease, post-streptococcal glomerulonephritis, or a variety of pediatric autoimmune neuropsychiatric disorders associated with streptococcal infections (PANDAS).

The presence of a cellular immune response is determined preferably after eliciting a reaction by administration of at least one of the autoantigens of the present invention. Using quantitative methods of the present invention it is possible to identify the most reactive autoantigen or most reactive epitope or most reactive group of autoantigens which can improve the treatment of the autoimmune disease.

According to a further embodiment, the present invention also provides a diagnosis method for detecting the presence of a cellular immune response in a patient (to at least one of the herein defined autoantigens, fragments, variants or epitopes thereof, wherein the method comprises the following steps:
(a) obtaining or providing a sample from a patient
(b) detecting a cellular immune response in the sample, by qualitatively and/or quantitatively detecting the secretion of at least one factor involved with a cellular immune response, using common detection methods, for example biophysical or biomolecular detection methods; and optionally
(c) detecting a humoral immune response, by qualitatively and/or quantitatively detecting in the sample at least one “autoreactive” antibody against one or more of the autoantigens of the present invention using common detection methods, for example biophysical or biomolecular detection methods and
(d) evaluating the results of step (b) and optionally step (c).

Preferably, the method is carried out in vitro but can also be used to detect an auto-immune response in vivo.

The present invention provides for any combination of steps and any option for detecting an immune response, i.e. detecting a cellular immune response, detecting a humoral immune response, detecting both responses, detecting first cellular immune response and then a humoral immune response or starting with the detection of a humoral immune response and thereafter detecting a cellular immune response.

The method as outlined according to the last mentioned alternative can be carried out by detecting as described in detail with respect to that method. The (diagnosis) method according to the third alternative can be, e.g., previously carried out by detecting a cellular immune response in the sample, preferably by qualitatively and/or quantitatively detecting the secretion of at least one factor involved with a cellular immune response, using biophysical or biomolecular detection methods (step (b)). This step was described previously as step (c) with respect to the second alternative of the inventive (diagnosis) method. Depending on the results obtained in that step or the sequential optional step (d), i.e. evaluating the results of step (b), the humoral immune response, preferably by qualitatively and/or quantitatively detecting in the sample at least one (“autoreactive”) antibody against one or more of the autoantigenic proteins Ezrin, Serpin B5, Peroxiredoxin-2 and heat shock protein beta-1, or a fragment, variant or epitope thereof, using biophysical or biomolecular detection methods, can be carried out in a further step (c). For example, if the results are positive and indicative of a cellular immune response, step (c) may be carried out, additionally.

In one embodiment in a first step the presence of autoreactive antibodies is detected, if the result is positive in a second step the presence of a cellular immune response against at least one of the autoantigens of the present invention is detected.

The evaluation of the results obtained with this method provides evidence if a GAS driven condition is present in a patient. The results obtained can be evaluated in usual manner. If antiautoantigen antibodies can be detected this is evidence for the presence of an autoimmune response against one of the autoantigens tested or in other words for the presence of a GAS driven condition. If a cellular immune response has been analyzed an increase of one or more factors involved in a cellular immune response after administration of an autoantigen or after contact with an autoantigen is evidence for a cellular autoimmune response to this or these autoantigens.

Based on the knowledge about the autoantigens of the present invention it is possible to provide treatment for GAS driven autoimmune diseases. Thus, in a further embodiment the present invention provides a method of treatment of diseases induced and/or mediated by group A beta-haemolytic streptococci, preferably selected from the group consisting of psoriasis (vulgaris), including all subtypes of psoriasis such as exanthematic guttate psoriasis, (chronic) plaque psoriasis, erythrodermic psoriasis, pustular psoriasis, psoriatic arthritis, etc., and several further autoimmune disorders including rheumatic fever and heart disease, post-streptococcal glomerulonephritis, or a variety of pediatric autoimmune neuropsychiatric disorders associated with streptococcal infections (PANDAS).

According to a first alternative, such a method of treatment is directed to desensitize or provide a tolerizing effect on the immune system of a patient to be treated with respect to excessive or exaggerated immune reactions due to mimicry, i.e. due to cross-reactive immune reactions, including the autoantigenic proteins Ezrin, Serpin B5, Peroxiredoxin-2 and/or Heat shock protein beta-1, or a fragment, variant or epitope thereof. In the context of the present inventive method, such a desensitizing or tolerizing effect on the immune system of a patient to be treated typically results in a less intense immune reaction towards these autoantigenic proteins or their fragments, variants or epitopes thereof.

The autoantigens of the present invention enable to analyze those epitopes or peptides or proteins that are responsible for the autoimmune reaction and thus, provide the possibility to target those unwanted antibodies in the immune system. Thus, the present invention contributes to a safer and more efficient treatment. In order to desensitize or hyposensitize the immune system or provide a tolerizing effect, the patient's immune system is preferably challenged with a “safe and effective amount” (within the meaning of the present invention) of at least one of the above mentioned described autoantigenic proteins, and/or a peptide comprising at least 5 consecutive amino acid residues of one of these autoantigenic proteins having immunological activity, or fragments, variants, or epitopes thereof and/or mixtures of at least two of the proteins and/or peptides, preferably contained in an inventive composition, or contained in an inventive pharmaceutical composition, both as disclosed herein. As a consequence the patient's immune system will provide an immune response, however, such an immune response may be small and controlled and thus may not cause any damage to the patient's immune system or health in general. The therapy may then be continued by stepwise and slightly increasing the “safe and effective amount” of at least one of the above mentioned described autoantigenic proteins, or of a fragment, variant or epitope thereof, until the patient's immune system is adapted to a considerable amount of the autoantigenic proteins, or of a fragment, variant or epitope thereof without leading to excessive or exaggerated immune reactions due to mimicry, i.e. due to cross-reactive immune reactions, including the autoantigenic proteins Ezrin, Serpin B5, Peroxiredoxin-2 and/or Heat shock protein beta-1, or a fragment, variant or epitope thereof.

The autoantigens (including fragments, variants or epitopes thereof) provide a valuable tool for desensitization or hyposensitization which will result in a better tolerance of the patient's immune system against the autoantigens. By providing the relationship between the autoantigens of the present invention and the GAS driven diseases the inventors have contributed not only a valuable tool but also valuable options for preparing compositions to treat GAS driven conditions.

A method using desenstization or hyposensitization is particularly suitable for such diseases selected from the group consisting of psoriasis (vulgaris), including all subtypes of psoriasis such as exanthematic guttate psoriasis, chronic plaque psoriasis, erythrodermic psoriasis, pustular psoriasis, psoriatic arthritis, etc., and several further autoimmune disorders including rheumatic fever and heart disease, post-streptococcal glomerulonephritis, or a variety of pediatric autoimmune neuropsychiatric disorders associated with streptococcal infections (PANDAS) as disclosed above. Without being bound to theory, such a desensitizing or tolerizing effect of the immune system of a patient according to the first alternative may be based on mechanisms which have have been proposed for the induction of antigen-specific immune tolerance. Some studies have been reported on peripheral tolerization of effector and memory T cells by low density of antigen in the periphery or by exposure to intravenous soluble antigen. Early studies suggested that CD8(+) “suppressor” T cells were important, however, it is now accepted that antigen-specific tolerance induction may involve either anergy/deletion of CD4(+) T cells, or the induction of regulatory CD4(+) T cells that produce IL-10 and/or TGF-beta. There may also be a role for CD4(+) CD25(+) T(reg), even if it is not yet clear as to how and when these different mechanisms operate. In this context, an aberrant activation of the T-cell receptor alone in mature T cells can produce a long-lived state of functional unresponsiveness, known as anergy. In vitro clonal T-cell anergy is induced in previously activated T cells or T-cell clones by restimulation through the T-cell receptor (TCR) in the absence of co-stimulatory signals. This suboptimal signalling produces long-lived effects, such as reduced proliferation and cytokine production. The ability of fed antigens to induce oral tolerance probably may also reflect their uptake by “quiescent” antigen-presenting cells in the intestine, with presentation to specific CD4(+) T cells in the absence of costimulation, or with the involvement of inhibitory costimulatory molecules. Dendritic cells in the Peyer's patches or mucosal lamina propria are the most likely antigen presenting cells involved and may be crucial for the induction of oral tolerance. Recent work identified important roles for linker for activation of T cells (LAT) palmitoylation, diacylglycerol (DAG) signalling, and transcription factors for the induction of both in vitro and in vivo T-cell anergy.

The autoantigens (including fragments, variants, epitopes, or derivatives thereof) can be used in a method of treatment of GAS driven conditions, for example the above mentioned diseases.

In a particularly preferred embodiment in a first phase the at least one autoantigen or fragment or epitope thereof, that is the causative principle of the condition is detected by using the above mentioned methods. In a second phase the optimal autoantigen or variant, fragment or epitope thereof is selected for treatment and optionally after a treatment period the “autoimmune state” of the patient can be determined again using the autoantigens including variants, fragments and epitopes of the present invention.

Thus, the present invention on the one hand provides a tool for diagnosis and on the other hand at the same time a tool for treating the disease diagnosed by the method of the present invention.

A composition useful in these methods can be any composition comprising one of the auto-antigens as defined before and can be prepared and provided with the knowledge of the person skilled in the art.

Preparing (or providing) an inventive composition or an inventive pharmaceutical composition or an inventive vaccine as defined according to the present invention according to step (a) of the first alternative of the inventive method of treatment typically comprises any method for preparing a composition, a pharmaceutical composition or a vaccine as defined herein or as known in the art by a skilled person, e.g. by mixing at least one of the autoantigenic proteins Ezrin, Serpin B5, Peroxiredoxin-2 and Heat shock protein beta-1, or a fragment, variant or epitope thereof, preferably in the above defined combinations, optionally by using a suitable buffer and/or ingredients as defined above. According to an alternative approach a composition as defined above may also be prepared or provided involving an in vitro expansion of regulatory or effector T cells using the autoantigenic proteins Ezrin, Serpin B5, Peroxiredoxin-2 and/or Heat shock protein beta-1, and/or a peptide comprising at least 5 consecutive amino acid residues of one of these autoantigenic proteins having immunological activity, or fragments, variants, or epitopes thereof and/or mixtures of at least two of the proteins and/or peptides, for stimulation of T cells obtained from patients, preferably from patients suffering from diseases induced and/or mediated by group A beta-haemolytic streptococci (GAS). These regulatory or effector T cell populations may then be transferred into the patient by intraveneous, intramuscular or subcutaneous injection or infusion in the next step (b).

Administration of an inventive composition or an inventive pharmaceutical composition or an inventive vaccine (or of regulatory or effector T cell populations expanded in vitro using the autoantigenic proteins as define above) according to step (b) of the first alternative of the inventive method of treatment typically occurs generally as defined above for inventive pharmaceutical compositions or inventive vaccines, e.g. orally, parenterally, by inhalation spray, topically, rectally, nasally, buccally, vaginally or via an implanted reservoir. More preferably, administration occurs as specifically defined above for vaccines, i.e. systemically or locally, more preferably via transdermal, oral, parenteral routes, including subcutaneous or intravenous injections and/or intranasal administration routes, and even more preferably via intradermale, transdermal, intramuscular or subcutaneous injection.

Administration of an inventive composition or an inventive pharmaceutical composition or an inventive vaccine according to step (b) of the first alternative of the inventive method of treatment typically occurs at least once, preferably more than once, e.g. 1-2 times, 2-5 times, 5-10 times or even more often in order to achieve a desensitizing or tolerizing effect on the immune system of the patient to be treated. The number of repetitions will typically depend on the type and severity of the disease to be treated and will also vary with the age and physical condition of the patient to be treated, the body weight, general health, sex, diet, time of administration, rate of excretion, drug combination, the activity of the specific autoantigenic protein employed, the duration of the treatment, the nature of the accompanying therapy, of the particular pharmaceutically acceptable carrier used, and similar factors, within the knowledge and experience of the accompanying doctor.

Moreover, another aspect of the present invention is the use of at least one autoantigen selected from Ezrin, Serpin B5, Peroxiredoxin-2 and Heat shock protein beta-1, or a fragment, variant or epitope thereof, for preparing a composition for desensitization in a GAS driven condition.

Moreover, the present invention provides for the use of at least one autoantigen selected from Ezrin, Serpin B5, Peroxiredoxin-2 and Heat shock protein beta-1, and/or a peptide comprising at least 5 consecutive amino acid residues of one of these autoantigenic proteins having immunological activity, or fragments, variants, or epitopes thereof and/or mixtures of at least two of the proteins and/or peptides, for preparing a composition for the detection of antibodies against at least one of the autoantigens or fragments or epitopes thereof.

In a second alternative a method of therapy of diseases induced and/or mediated by group A beta-haemolytic streptococci (GAS) is provided wherein this method is directed to ameliorating and/or diminishing the symptoms of these diseases by decreasing the number of (“autoreactive”) antibodies, directed against at least one of the herein defined autoantigenic proteins Ezrin, Serpin B5, Peroxiredoxin-2 and Heat shock protein beta-1, or a fragment, variant or epitope thereof. Decreasing the number of those (“autoreactive”) antibodies may, e.g., be aimed at the level of the B cells or plasma cells that are producing those (“autoreactive”) antibodies as described above, e.g. by reducing the antibody production of these cells or by eliminating those B cells or plasma cells. Decreasing the number of those (“autoreactive”) antibodies may, e.g., also be aimed by binding or blocking those (“autoreactive”) antibodies, e.g. by a secondary antibody such as an anti-idiotypic antibody as defined herein or by one or more of the autoantigenic proteins Ezrin, Serpin B5, Peroxiredoxin-2 and Heat shock protein beta-1, or a fragment, variant or epitope thereof or by any other compound suitable for binding or blocking those (“autoreactive”) antibodies, and optionally by removing the bound or blocked (“autoreactive”) antibody from the patient's circular system, e.g., by natural mechanisms or by means of a haemodialysis or other suitable methods known to a skilled person. Such a method of therapy may thus utilize administration of an inventive antibody composition. Preferably, the antibodies of such an inventive antibody composition are selected from anti-idiotypic antibodies or antibody fragments as defined above, capable of recognizing at least one (primary) antibody selectively binding to one of the autoantigenic proteins Ezrin, Serpin B5, Peroxiredoxin-2 and Heat shock protein beta-1, or a fragment, variant or epitope thereof, as defined above. More preferably, the (at least one) anti-idiotypic antibody or a fragment thereof is directed against at least one (preferably different) (“autoreactive”) antibody or a fragment thereof, each of them recognizing a preferably different autoantigenic protein as defined above, or a fragment, variant or epitope thereof. Thus, each anti-idiotypic antibody species has preferably a different (“autoreactive”) antibody target, which, in turn, recognizes at least one (preferably different) autoantigenic protein. Alternatively, the (at least one) anti-idiotypic antibody or a fragment thereof may be a bispecific antibody, which is directed on one hand side to one (“autoreactive”) antibody or a fragment thereof, recognizing an autoantigenic protein as defined above, or a fragment, variant or epitope thereof, and on the other hand side to a group for immobilization on a solid phase, which allows binding of the (bispecific) anti-idiotypic antibody or a fragment thereof to a sample carrier, support, or matrix. According to a further alternative, the (at least one) anti-idiotypic antibody or a fragment thereof may also be a bispecific antibody which already contains such a group and can be immobilized on a sample carrier, support, or matrix. Such an (at least one) bispecific anti-idiotypic antibody or a fragment thereof may further recognize at least one (“autoreactive”) antibody directed against an autoantigenic protein as defined above, or a fragment, variant or epitope thereof, preferably directed against a combination selected from e.g. the autogenic proteins Ezrin and Serpin B5, Ezrin and Peroxiredoxin-2, Ezrin and Heat shock protein beta-1, Serpin B5 and Peroxiredoxin-2, Serpin B5 and Heat shock protein beta-1, or Peroxiredoxin-2 and Heat shock protein beta-1. Additionally, any of the above methods for decreasing the number of (“autoreactive”) antibodies may be combined with each other in the inventive method of treatment.

B-specific antibodies are particularly suitable for screening and removing autoantigens in vitro.

According to one embodiment that is useful in a method of therapy of diseases induced and/or mediated by group A beta-haemolytic streptococci (GAS) particlularly primary and/or secondary diseases, a composition for the administration of at least one antibody or antibody fragments, directed against at least one of the herein defined autoantigenic proteins Ezrin, Serpin B5, Peroxiredoxin-2 and Heat shock protein beta-1, or a fragment, variant or epitope thereof is provided. According to the invention, the antibody may be a natural antibody or a genetically manipulated antibody. The antibody binds preferably to at least one of the autoantigenic proteins Ezrin, Serpin B5, Peroxiredoxin-2 and Heat shock protein beta-1, or a fragment, variant or epitope thereof and renders those proteins, fragments, variants or epitopes thereof not accessible to naturally produced antibodies in the body and/or blocks the humoral immune response to those autoantigenic compounds.

In a preferred embodiment fragments of antibodies are used, particularly F(ab) fragments or F(ab′)₂ fragments are used for the treatment of GAS driven conditions where the missing Fc parts avoids eliciting of an immune response agaisnt the antibody. This is particularly useful for the treatment of glomerulo nephritis.

In another embodiment complexes of at least one autoantigen of the present invention and antibodies or fragments thereof binding with the autoantigen are provided and can be used to elicit receptor mediated immune reactions.

According to an embodiment, the present invention also provides the use of at least one of the autoantigenic proteins Ezrin, Serpin B5, Peroxiredoxin-2 and Heat shock protein beta-1, and/or a peptide comprising at least 5 consecutive amino acid residues of one of these autoantigenic proteins having immunological activity, or fragments, variants, or epitopes thereof and/or mixtures of at least two of the proteins and/or peptides, for the preparation of a(n inventive) composition, a(n inventive) pharmaceutical composition or a (n inventive) vaccine or an inventive antibody composition in any of the above methods.

Particularly the present invention provides the use of at least one of the autoantigens of the present invention as defined above, or any sequence of an autoantigen that is immunologically active, particularly any sequence as defined in this description for preparing a composition for detecting or treating an GAS driven condition.

The present invention provides the use of at least one of the autoantigenic proteins Ezrin, Serpin B5, Peroxiredoxin-2 and Heat shock protein beta-1, or of a variant, fragment or epitope thereof, (for the preparation of a(n inventive) composition, a(n inventive) pharmaceutical composition or a(n inventive) vaccine or an inventive antibody composition) for the treatment of (primary and/or secondary) diseases induced and/or mediated by group A beta-haemolytic streptococci (GAS), preferably selected from the group consisting of psoriasis (vulgaris), including all subtypes of psoriasis such as exanthematic guttate psoriasis, chronic plaque psoriasis, erythrodermic psoriasis, pustular psoriasis, psoriatic arthritis, etc., and several further autoimmune disorders including rheumatic fever and heart disease, post-streptococcal glomerulonephritis, or a variety of pediatric autoimmune neuropsychiatric disorders associated with streptococcal infections (PANDAS). Preferably, such a treatment is directed to desensitizing or tolerizing the immune system of a patient to be treated with respect to excessive or exaggerated immune reactions due to mimicry, i.e. due to cross-reactive immune reactions, including the autoantigenic proteins Ezrin, Serpin B5, Peroxiredoxin-2 and Heat shock protein beta-1, or a fragment, variant or epitope thereof.

Particularly useful are also the sequences disclosed in this description.

Moreover, the autoantigens, fragments, variants and/or epitopes thereof are also useful for monitoring the course of the disease during any desensitizing, hyposensitizingor otherwise relieving treatment.

According to a further embodiment, the present invention also provides the use of at least one of the autoantigenic proteins Ezrin, Serpin B5, Peroxiredoxin-2 and Heat shock protein beta-1, and/or a peptide comprising at least 5 consecutive amino acid residues of one of these autoantigenic proteins having immunological activity, or fragments, variants, or epitopes thereof and/or mixtures of at least two of the proteins and/or peptides, for the preparation of a(n inventive) composition) and/or an inventive antibody composition for the diagnosis of (primary and/or secondary) diseases induced and/or mediated by group A beta-haemolytic streptococci (GAS), preferably selected from the group consisting of psoriasis (vulgaris), including all subtypes of psoriasis such as exanthematic guttate psoriasis, chronic plaque psoriasis, erythrodermic psoriasis, pustular psoriasis, psoriatic arthritis, etc., and several further autoimmune disorders including rheumatic fever and heart disease, post-streptococcal glomerulonephritis, or a variety of pediatric autoimmune neuropsychiatric disorders associated with streptococcal infections (PANDAS). More preferably, the present invention also provides the use of at least one of the autoantigenic proteins Ezrin, Serpin B5, Peroxiredoxin-2 and Heat shock protein beta-1, and/or a peptide comprising at least 5 consecutive amino acid residues of one of these autoantigenic proteins having immunological activity, or fragments, variants, or epitopes thereof and/or mixtures of at least two of the proteins and/or peptides, for the preparation of a(n inventive) composition for detecting the presence of a humoral and additionally a cellular immune response in a patient (to at least one of the herein defined autoantigenic proteins Ezrin, Serpin B5, Peroxiredoxin-2 and Heat shock protein beta-1, and/or a peptide comprising at least 5 consecutive amino acid residues of one of these autoantigenic proteins having immunological activity, or fragments, variants, or epitopes thereof and/or mixtures of at least two of the proteins and/or peptides, preferably according to the inventive diagnosis method as defined above.

According to another embodiment, the present invention also provides the use of at least one antibody as defined herein (for the preparation of a(n inventive) antibody composition or a(n inventive) pharmaceutical composition) for the treatment of (primary and/or secondary) diseases induced and/or mediated by group A beta-haemolytic streptococci (GAS), preferably selected from the group consisting of psoriasis, including psoriasis vulgaris (plaque psoriasis), erythrodermic psoriasis, pustular psoriasis, psoriatic arthritis, etc., and several further autoimmune disorders including rheumatic fever and heart disease, post-streptococcal glomerulonephritis, or a variety of pediatric autoimmune neuropsychiatric disorders associated with streptococcal infections (PANDAS). Preferably, such an antibody may either be directed against at least one of the autoantigenic proteins Ezrin, Serpin B5, Peroxiredoxin-2 and Heat shock protein beta-1, or, even more preferably, may be an anti-idiotypic antibody, directed against an such an antibody, selectively binding at least one of the autoantigenic proteins Ezrin, Serpin B5, Peroxiredoxin-2 and Heat shock protein beta-1. Preferably, such a treatment is directed to ameliorating and/or diminishing the symptoms of these diseases by decreasing the number of those antibodies, directed against at least one of the herein defined autoantigenic proteins Ezrin, Serpin B5, Peroxiredoxin-2 and Heat shock protein beta-1, or a fragment, variant or epitope thereof.

Furthermore the present invention provides kits, particularly kits of parts, comprising as components the inventive composition and/or the inventive antibody and/or the inventive composition and/or the inventive pharmaceutical composition and/or the inventive vaccine, and optionally technical instructions with information on the administration and dosage of these components. The technical instructions may contain information about administration and dosage of the inventive composition, and/or the antibody, and/or the inventive pharmaceutical composition and/or the inventive vaccine. Such kits, preferably kits of parts, may applied e.g. for any of the above mentioned methods of treatment or uses, particularly in the treatment of (primary and/or secondary) diseases induced and/or mediated by group A beta-haemolytic streptococci (GAS), preferably selected from the group consisting of psoriasis (vulgaris), including all subtypes of psoriasis such as exanthematic guttate psoriasis, chronic plaque psoriasis, erythrodermic psoriasis, pustular psoriasis, psoriatic arthritis, etc., and several further autoimmune disorders including rheumatic fever and heart disease, post-streptococcal glomerulonephritis, or a variety of pediatric autoimmune neuropsychiatric disorders associated with streptococcal infections (PANDAS). Kits of parts, as a special form of kits, may be e.g. used, when a time staggered treatment is envisaged, wherein the single parts of such a kit may contain either the same or different components as defined above.

According to a further aspect the present invention provides a test kit comprising

(a) at least one autoantigen as defined above, optionally immobilized on a carrier;

(b) a container for receiving a sample to be analyzed and optionally

(c) carriers and excipients, and a buffering agent for adapting the pH.

The test kit is particularly useful for the detection of autoreactive antibodies and/or the diagnosis of a GAS driven condition. The test kit can comprise at least one autoantigen or a variant, fragment, or epitope thereof as defined before or a combination of different variants, fragments, epitopes, peptides or whole proteins. In one preferred embodiment a “cocktail” of different epitopes or variants, fragments, peptides or proteins having different eptiopes is contained to allow a screening for autoreactive antibodies. In another preferred embodiment different epitopes of one protein or a mixture of different epitopes of different proteins can be part of the kit.

The container for receiving the sample and, thereafter, for mixing the necessary reagents, is a container as known in the art and well known to the skilled person. Optionally the test kit also contains a buffering agent and/or further excipients as it is well known for test kits for detecting analytes.

Summarizing the present invention provides a diagnostic tool to determine the presence of a streptococcal driven condition. By providing this diagnostic tool it is possible to define a disease state and to find the best therapeutic agent. Moreover, the present invention provides a therapeutical composition for treating autoimmune diseases that have been elicited by a primary infection with streptococcae. Moreover, methods for detecting the presence of specific antibodies, presence of autoimmune responses and method for exactly designing a composition to treat autoimmune diseases are provided.

### FIGURES

The following Figures are only illustrative to the present invention and shall describe particular embodiments of the present invention in further detail. However, these Figures are not intended to limit the subject matter of the present invention thereto.

FIG. 1: shows the results of Western immunoblotting of serological reactivities with keratinocyte proteins and staining of proteins using the pre-immune rabbit sera and the streptococci-specific rabbit sera. The proteins were fractionated by 2D-gel electropheresis. The gel shows:
A: rabbit pre-immune serum;
B. Coomassie stain
C: rabbit streptococci-specific hyper-immune sera;
D: psoriasis patient serum.

Circles in FIGS. 1C and 1D indicate overlapping=identical reactivities of the hyper-immune serum and patient serum corresponding to the proteins Serpin B5, Ezrin, Peroxiredoxin-2, Heat shock protein beta-1, keratin 6.

As can be seen, the pre-immune rabbit sera stained only a few keratinocyte proteins, with a major reactivity against a protein that, according to its position within the corresponding Coomassie-stained gel, represented human actin. The streptococci-specific rabbit sera instead stained several additional keratinocyte proteins that definitely were not seen with the pre-immune sera.

FIG. 2: depicts the stimulation results (ELISPOT assay) of psoriasis patients. PBMCs (Peripheral Blood Mononuclear Cells) of 76 psoriasis patients and 22 healthy controls were stimulated with the different proteins, and the resulting T-cell activation was determined as IFN-gamma-producing T cells. HspB1=Heat shock protein beta-1; PHA=phytohaemagglutinin. Mean values plus SD (vertical bars). The single FIGS. 2A-J show the T-cell response to the specifically indicated proteins in the different cohorts of psoriasis patients or healthy controls:

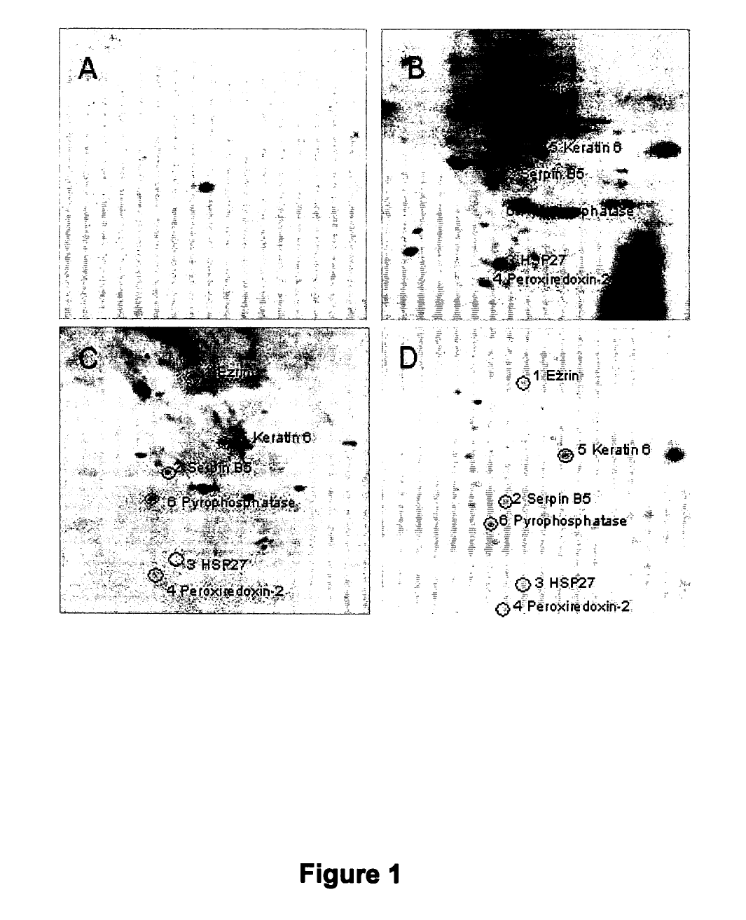

FIG. 2A: negative control

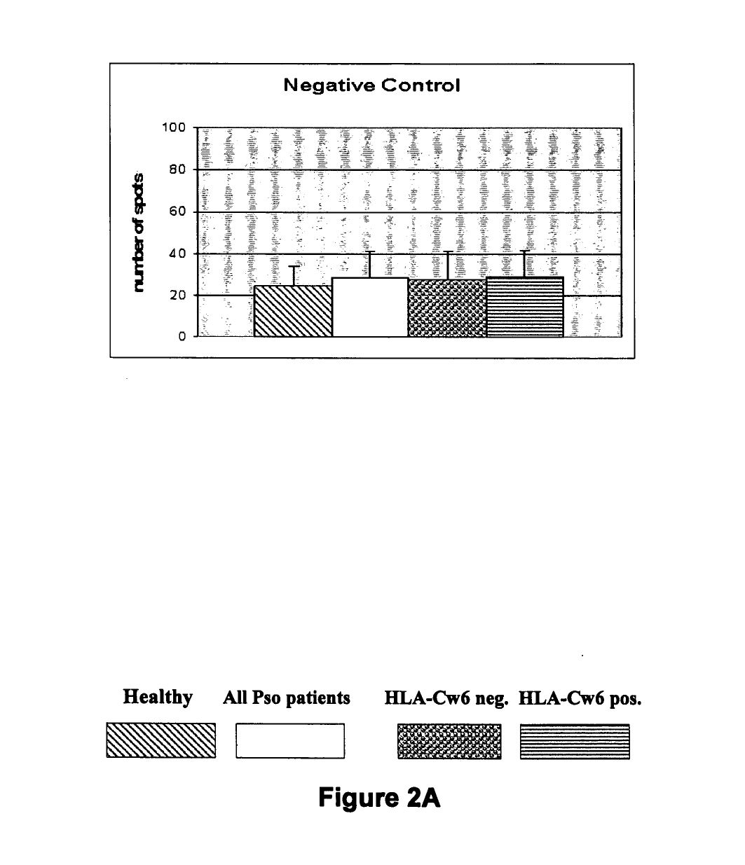

FIG. 2B: Hsp B1 (Heat shock protein beta-1)

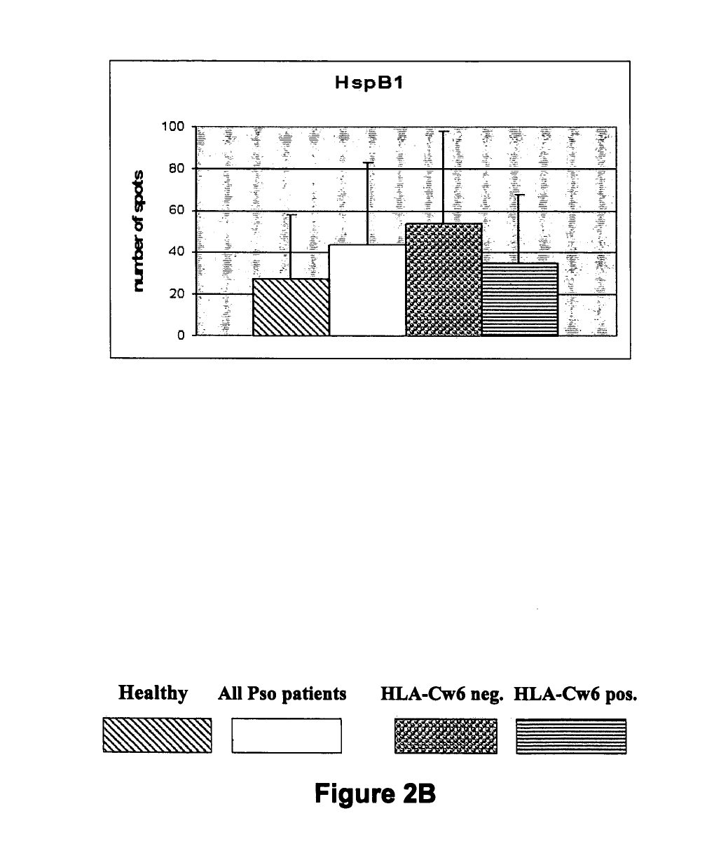

FIG. 2C: Ezrin

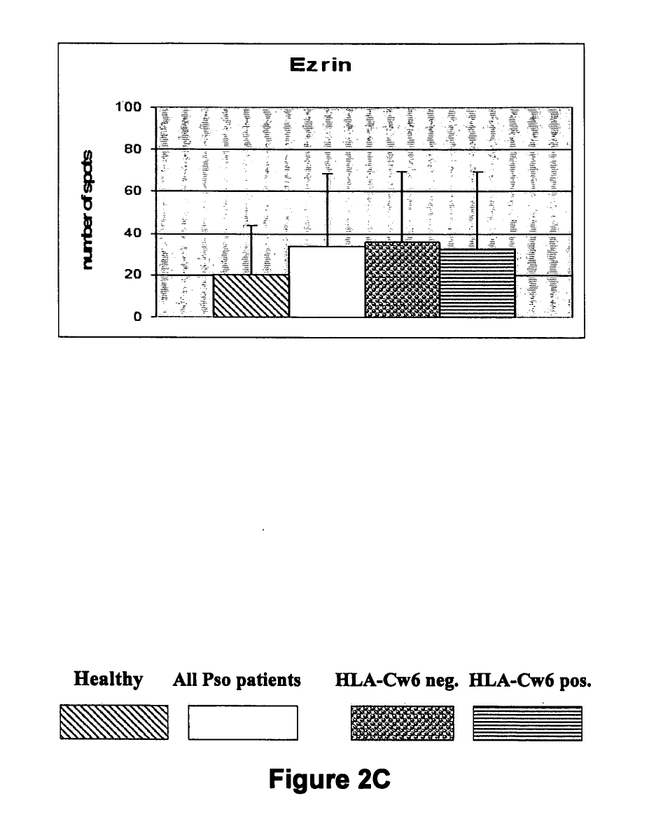

FIG. 2D: Serpin B5

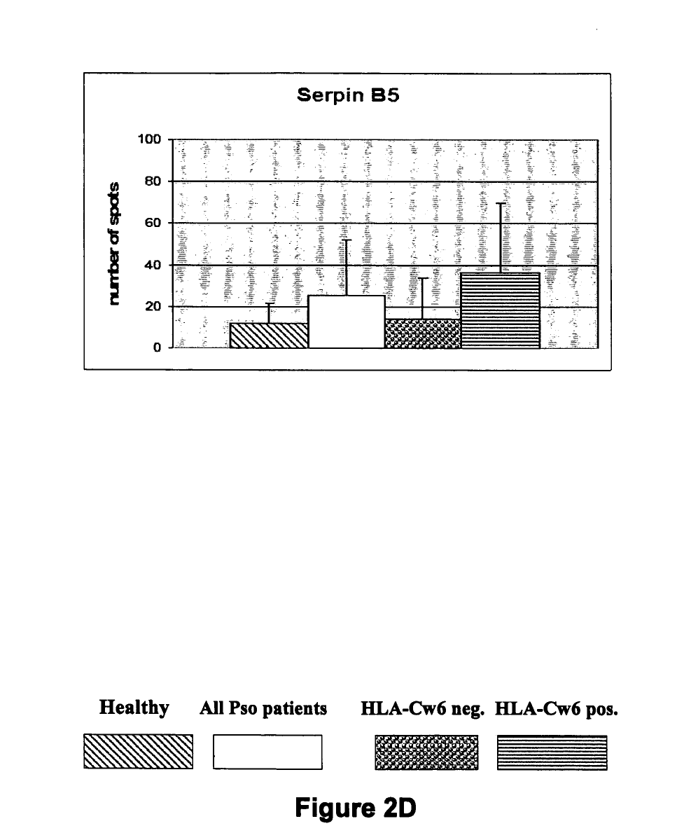

FIG. 2E: Peroxiredoxin II (Peroxiredoxin-2)

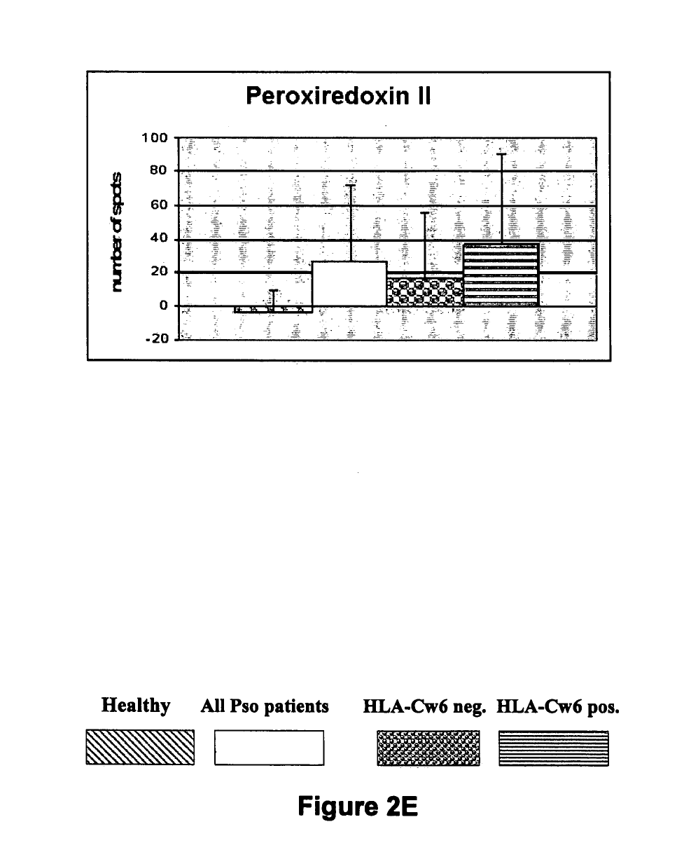

FIG. 2F: Keratin 6 (N-terminal KvH)

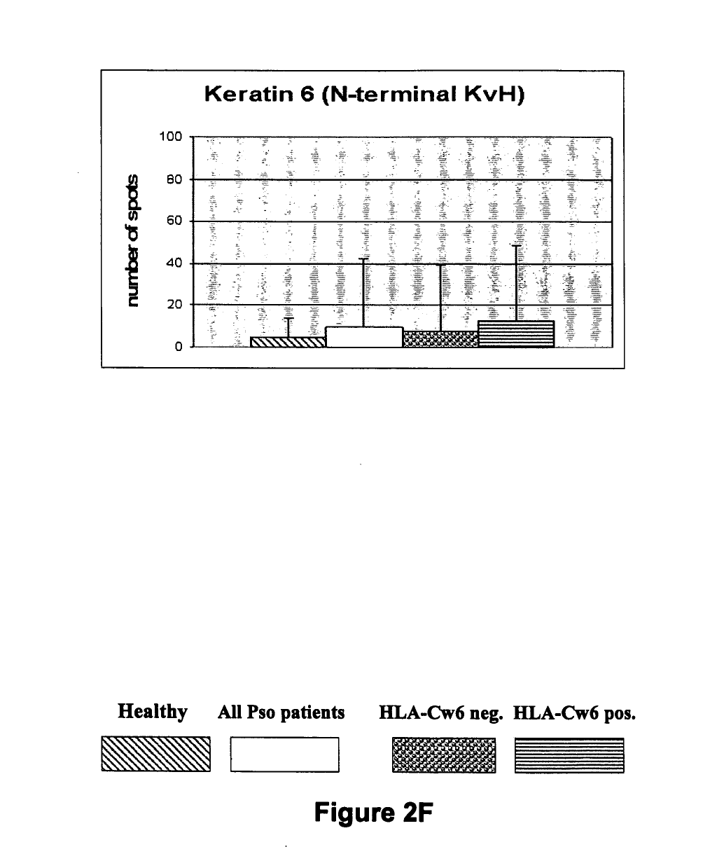

FIG. 2G: Keratin 6 (C-terminal KhF)

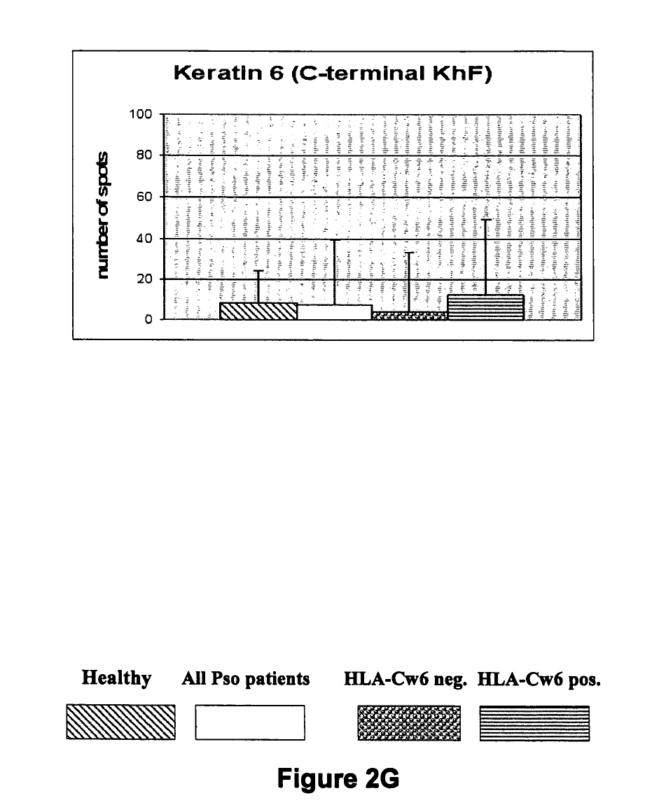

FIG. 2H: Cytokeratin

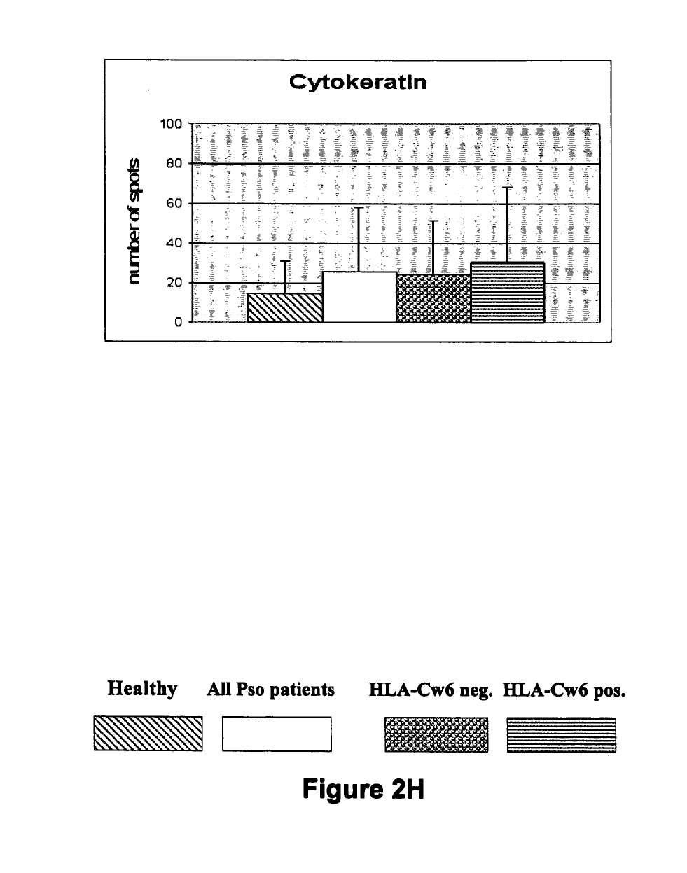

FIG. 2I: Tetanus toxoid

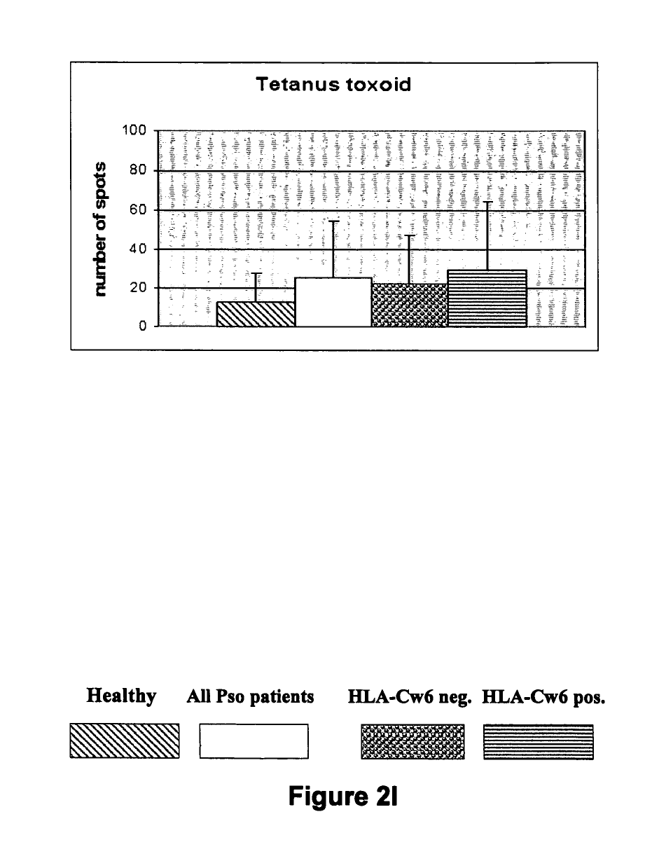

FIG. 2J: PHA (positive control)

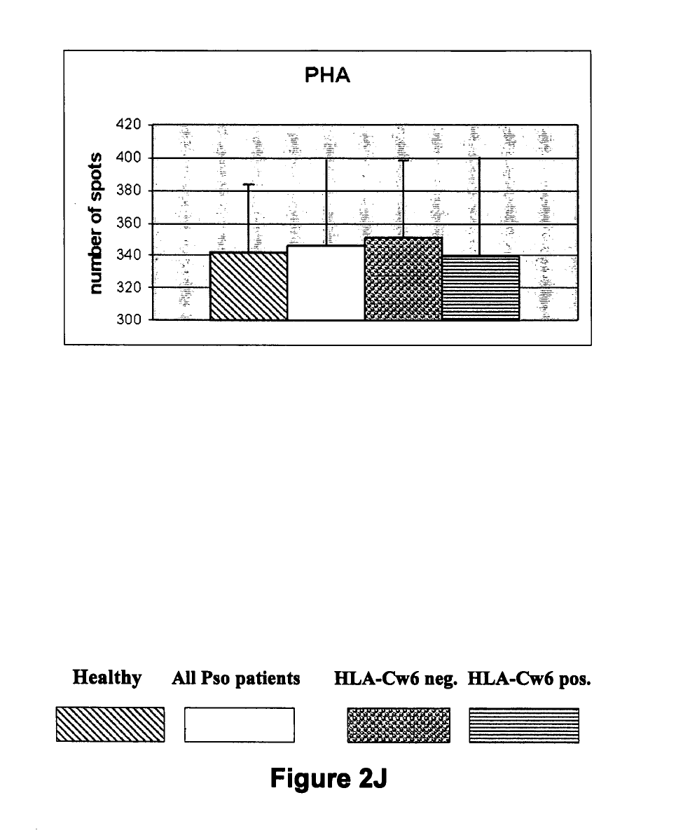

All FIGS. 2A-J show from left to right: healthy patients (N=22), all PSO patients (N=76), HLA-Cw6 neg. (N=36) and HLA-Cw6 pos. (N=38).

FIG. 3: depicts the sequence of Ezrin (Name of protein: Ezrin; Synonyms: p81, Cytovillin, Villin-2; Name of gene: VIL2; from Homo sapiens (TaxID: 9606); GI-Nummer: GI 31282; UniProtKB/Swiss-Prot entry P15311); (see SEQ ID NO: 1);

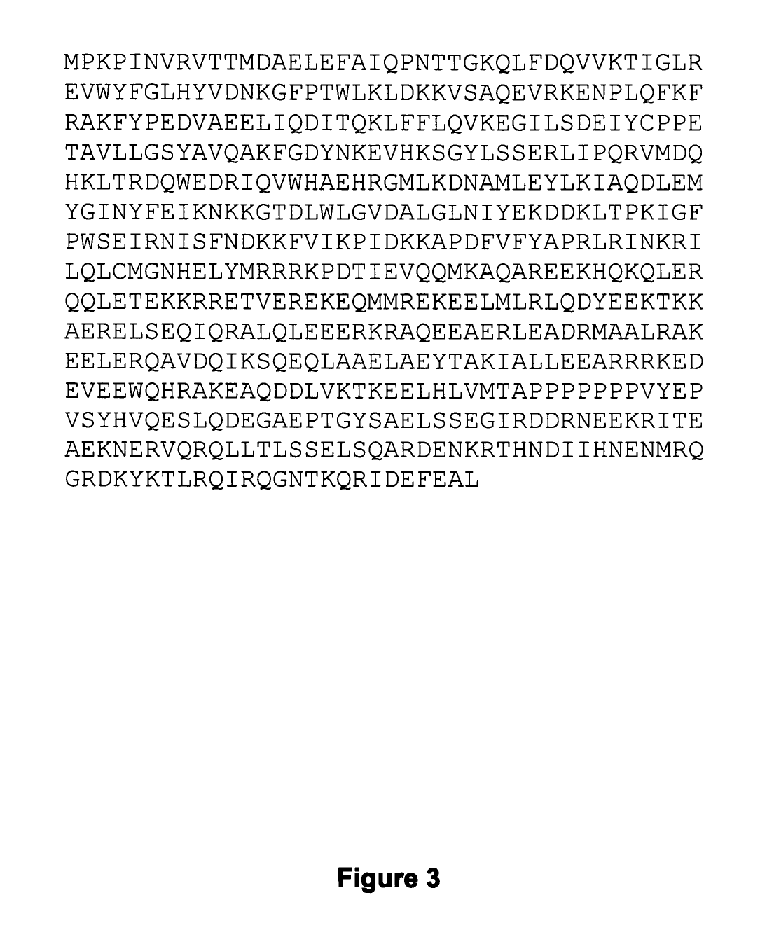

FIG. 4: shows the sequence of Serpin B5 (Name of protein: Serpin B5 [Precursor]; Synonyms: Maspin, Protease inhibitor 5; Name of gene: SERPINB5; Synonyms: P15; from Homo sapiens (TaxID: 9606); GI-Nummer: GI 142377273; UniProtKB/Swiss-Prot entry P36952); (see SEQ ID NO: 2);

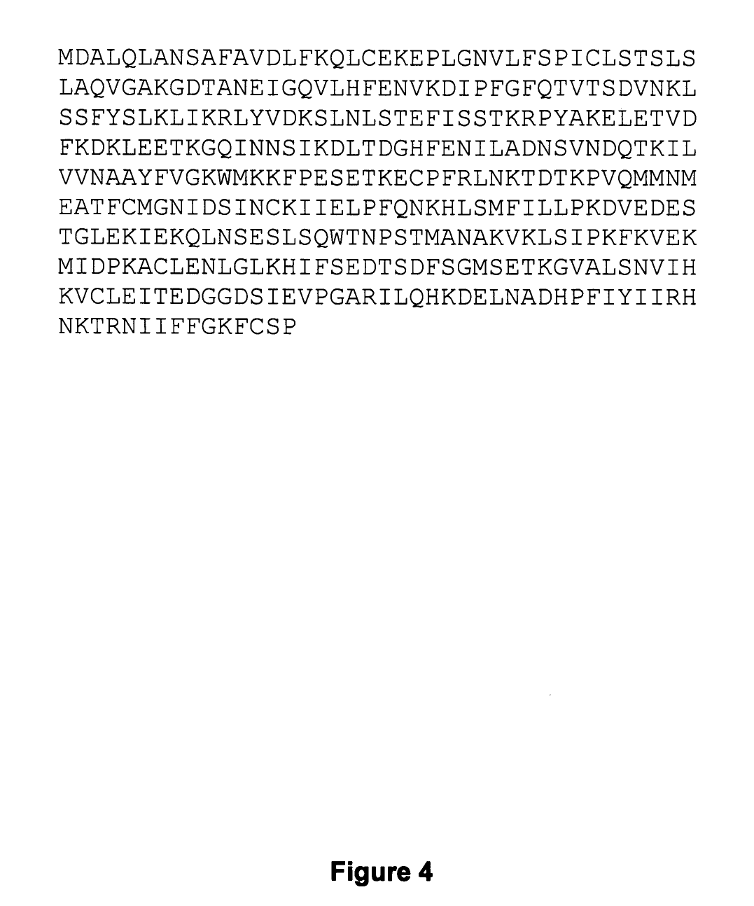

FIG. 5: provides the sequence of Peroxiredoxin-2 (Name of protein: Peroxiredoxin-2; Synonyms: EC 1.11.1.15, Thioredoxin peroxidase 1, Thioredoxin-dependent peroxide reductase 1, Thiol-specific antioxidant protein TSA, PRP; Name of gene: PRDX2; Synonyms: TDPX1; from Homo sapiens (TaxID: 9606); GI-Nummer: GI440307; UniProtKB/Swiss-Prot entry P32119); (see SEQ ID NO: 3);

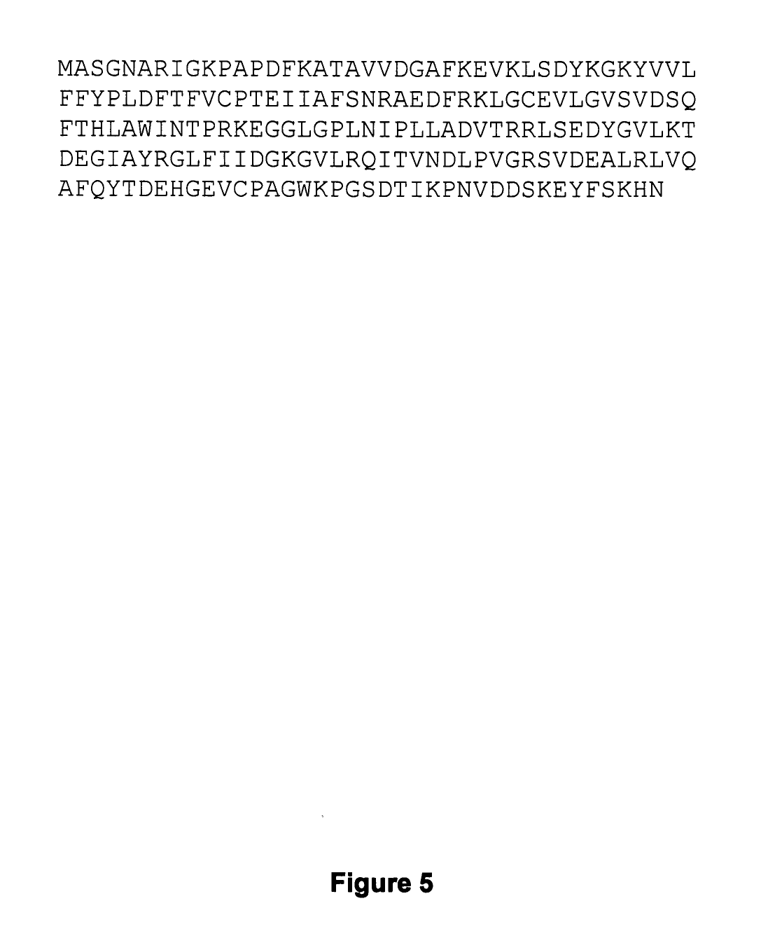

FIG. 6: shows the sequence of Heat shock protein beta-1 (Name of protein: Heat shock protein beta-1; Synonyms: HspB1, Heat shock 27 kDa protein,

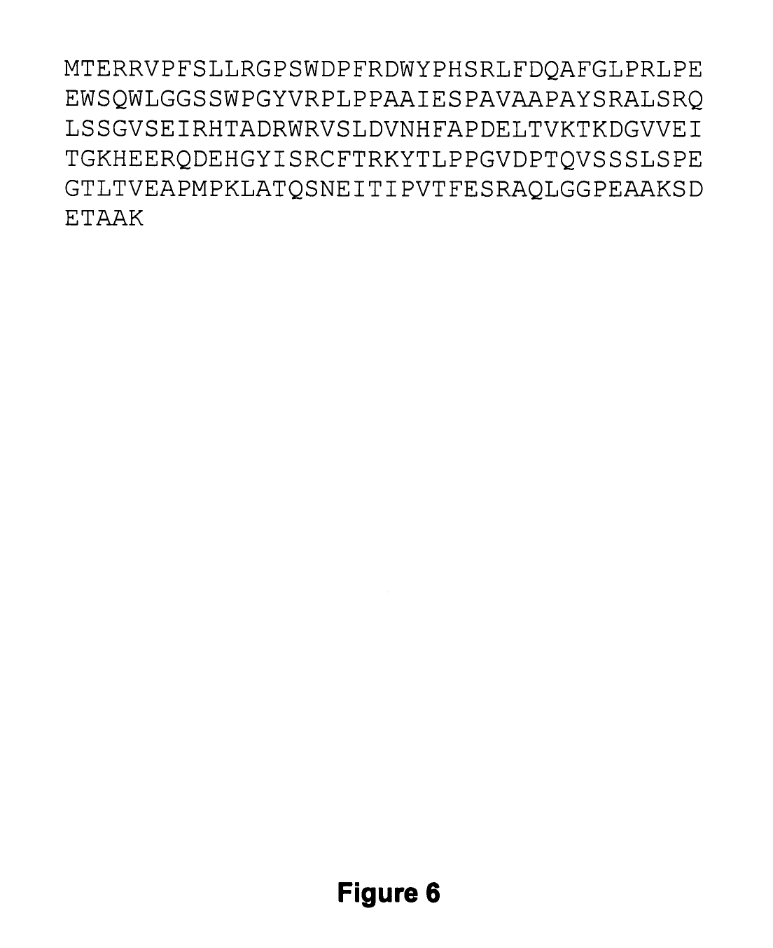

HSP 27, Stress-responsive protein 27, SRP27, Estrogen-regulated 24 kDa protein, 28 kDa heat shock protein; Name of gene: HSPB1; Synonyms HSP27; from Homo sapiens (TaxID: 9606); GI-Nummer: GI32477; UniProtKB/Swiss-Prot entry PO4792); (see SEQ ID NO: 4);

FIG. 7: shows the results of an ELISPOT analysis as described in Example 2.

### EXAMPLES

### Example 1

The following Examples are only illustrative to the present invention and shall describe particular embodiments of the present invention in further detail. However, these Examples are not intended to limit the subject matter of the present invention thereto.

1. Infection and Immunization of Rats with Group A β-haemolytic Streptococci (GAS)

In our approach and experiments to identify the putative psoriatic autoantigens we have made use of the fact that angina caused by group A β-haemolytic streptococci (GAS) is the most common trigger of psoriasis onset or flares. In order to identify psoriatic autoantigens based on molecular mimicry we immunized rabbits with group A β-haemolytic streptococci (GAS). More particularly, to identify common epitopes on streptococcal antigens and keratinocyte proteins, rabbits were repeatedly immunized with S. pyogenes, serotype M1 and M12. These serotypes had been chosen because they had frequently been isolated from the throat of patients with streptococcal-induced psoriasis.

This immunization induced antibodies against several keratinocyte proteins that were also recognized by sera from psoriasis patients. These proteins were considered as potential targets of a cross-reactive anti-streptococcal immune response in psoriasis. When used for the stimulation of peripheral blood mononuclear cells from psoriasis patients in vitro they induced a pronounced activation and oligoclonal expansion of T cells that employed TCR rearrangements similar to those expanded within the psoriatic skin lesions.

Therefore, these proteins were considered by us to actually represent antigenic targets of the psoriatic T-cell response.

2. Detection of Reactivity of Streptococci-Specific Rabbit or Patient Sera with Keratinocyte Proteins

Subsequent to immunization of rabbits with S. pyogenes, serotype M1 and M12 and antibody formation according to Example 1, the antibody reactivities of the pre- and hyperimmune rabbit sera with lysates of human keratinocytes, a lymphoblastoid B-cell line, and the epidermoid carcinoma line, A431, were fractionated by SDS-PAGE and determined by Western-Immunoblotting.

Comparison with the pre-immune rabbit sera demonstrated that streptococcal immunization gave rise to a number of serologic reactivities with the keratinocytes lysates that became visible as stained bands. The antibodies reactivities appeared to be specific for keratinocyte proteins, since no obvious reactivities against lysates of an EBV-transformed lymphoblastoid B-cell line or the epidermoid carcinoma cell line, A431, had been induced. They could largely be absorbed by incubation with lysates from the streptococci used for immunization, but not E. coli. Thus, the immunological challenge of the rabbits with streptococci had induced an antibody formation against various keratinocyte proteins that surprisingly seemed to involve cross-reactive anti-streptococcal antibodies.

3. Identification of Keratinocyte Proteins Recognized by both Streptococci-Specific Hyper-Immune Sera and Sera from Psoriasis Patients

To identify the keratinocyte proteins represented by the bands obtained by SDS-PAGE we analysed the antibody reactivities of sera from rabbits immunized according to Example 1, and alternatively from psoriasis patients or from healthy individuals with keratinocyte protein lysates, wherein the protein bands had been fractionated by 2D-SDS gel electrophoresis according to Example 2.

Furthermore the pre-immune rabbit sera were determined by Western immunoblotting and stained only a few keratinocyte proteins, with a major reactivity against a protein that, according to its position within the corresponding Coomassie-stained gel, represented human actin. The streptococci-specific rabbit sera instead stained several additional keratinocyte proteins that definitely were not seen with the pre-immune sera (see FIG. 1).

Sera of the psoriasis patients reacted with various keratinocyte proteins. Interestingly, seven keratinocyte proteins were stained by antibodies from both the patients' sera (n=5) and the streptococci-specific rabbit sera but not the pre-immune rabbit sera. Because in the rabbits these antibody reactivities had obviously been induced by streptococcal immunization we considered the corresponding keratinocyte proteins as potential targets of a cross-reactive anti-streptococcal immune response that might be relevant in psoriasis. To identify them the corresponding protein spots were cut out from Coomassie-stained two-dimensional polyacrylamide keratinocyte protein gels, digested with protease Endo Lys C in the gel, and the resulting peptides were eluted and separated by HPLC. The amino-acid sequence of peptides from each protein spot was determined by Edman degradation in an automatic peptide analyzer (table 1). By alignments with the Swissprot protein sequence library using the programs of the Fast- and Blast-family, the amino acid sequences of all analyzed peptides could unambiguously be assigned to the primary structure of proteins for which the amino-acid sequence had already been determined. These proteins were
Keratin 6
Ezrin; Synonyms: p81, Cytovillin, Villin-2; Gene name: VIL2; from Homo sapiens (TaxID: 9606); GI-Nummer: GI 31282; UniProtKB/Swiss-Prot entry P15311
Serpin B5 [Precursor]; Synonyms: Maspin, Protease inhibitor 5; Gene name: SERPINB5; Synonyms: P15; from Homo sapiens (TaxID: 9606); UniProtKB/Swiss-Prot entry P36952
Peroxiredoxin-2; Synonym: EC 1.11.1.15, Thioredoxin peroxidase 1, Thioredoxin-dependent peroxide reductase 1, Thiol-specific antioxidant protein
   TSA, PRP, Natural killer cell-enhancing factor B, Gene name: PRDX2; Synonym: TDPX1; from Homo sapiens (TaxID: 9606); GI-Nummer: GI440307; UniProtKB/Swiss-Prot entry P32119
Heat shock protein beta-1; Synonyms: HspB1, Heat shock 27 kDa protein, HSP 27, Stress-responsive protein 27, SRP27, Estrogen-regulated 24 kDa protein, 28 kDa heat shock protein; Gene name: HSPB1; Synonyms HSP27; from Homo sapiens (TaxID: 9606); GI-Nummer: GI32477; UniProtKB/Swiss-Prot entry PO4792

TABLE 1
Amino-acid sequence of the peptides sequenced from the
protein spots isolated from the Coomassie stained 2D-SDS
PAGE gels and the name and biochemical properties of
the identified corresponding human proteins

| | | Mole- | |
|---|---|---|---|
| Name of the | Amino acid sequence of the sequenced | cular | Iso- |
| identified | peptide and position in the primary | weight | electric |
| protein | structure of the identified protein | (kD) | point |
| Ezrin | AA 23 GFPTWLK AA 29 | 90 | 6.2-6.6 |
| | AA 162 LTRDQWEDRIQV AA 173 | | |
| | AA 426 KIALLEEARRKEDEVEEW AA 444 | | |
| Heat shock | AA 172 LATQSNEITIPFTFESRAQ AA 190 | 28 | 6.1 |
| protein beta-1 | | | |
| Keratin 6, | AA 360 YEELQITAGR AA 369 | 60 | 7.0 |
| Serpin B5 | AA 159 KILVVNAAYFVGK AA 170 | 45 | 6.1 |
| (precursor) | | | |
| Peroxi- | AA 120 DEGIAYRGLFIIDGK AA 135 | 21 | 5.9 |
| redoxin-2 | | | |

4. Stimulation Experiments

In order to determine the potential role of the proteins identified in Example 3 as autoantigens of the lesional psoriatic T-cell response we analyzed their ability to activate T cells from psoriasis patients and healthy controls. For this purpose the cDNA of Peroxiredoxin-2, Ezrin, Serpin B5, and of Keratin 6 were cloned into expression vectors and produced as recombinant proteins. Because of the protein size Keratin 6 was expressed as two overlapping peptides corresponding to the amino acids 10-200 and 189-503 of the Keratin 6f isoform. Recombinant heat shock protein beta-1 was purchased commercially.

Subsequently, PBMCs of 76 patients with chronic plaque psoriasis and 22 healthy individuals without a family history for psoriasis were cultured in vitro with the recombinant proteins. T-cell stimulation was determined by Elispot assay identifying IFN-gamma producing cells. Results were expressed as number of spots per 1.5×10⁵ PBMC and compared statistically. Psoriasis patients were differentially exploited according to the expression of HLA-Cw6 which is the major risk allele for psoriasis and present in the majority of type 1 psoriasis patients (early disease onset, positive family history for psoriasis). It was present in 38/74 of the psoriasis patients. PHA-stimulation served as positive control.

The results are given in FIG. 1. Baseline activation and PHA-stimulation were similar in psoriasis patients and healthy controls (A). As compared to healthy controls two of the proteins, Peroxiredoxin-2 (p=0.0003, Table 2) and Serpin B5 (p=0.0091, Table 2), induced a significantly increased T-cell stimulation in psoriasis patients. Their antigenicity appeared to be higher for HLA-Cw6 positive (Peroxiredoxin-2: p=0.0006; Serpin B5: p=0.0026) than for HLA-Cw6 negative patients (Peroxiredoxin-2: p=0.0301; Serpin B5: p=0.3768). Heat shock protein beta-1 induced a significantly increased stimulation in HLA-Cw6 negative patients (p=0.0307). Ezrin (p=0.060) and a cytokeratin preparation from keratinocytes (p=0.0641) tended to result in a higher T-cell activation in psoriasis patients than in healthy controls, but the difference did not reach statistical significance. No persistent T-cell stimulation could be induced by stimulation with the overlapping keratin 6-peptides, although individual patients responded quite strongly. These results suggest that
Peroxiredoxin-2 acts as an autoantigen of a T-cell mediated psoriatic immune response in the overall psoriasis patient population
Serpin B5 also acts as an autoantigen, preferentially for HLA-Cw6 positive psoriasis patients
Heat shock protein beta-1 and Ezrin act as an autoantigen and might be more relevant as target antigens of the pathogenic T-cell response in HLA-Cw6 negative patients
Keratin 6 does not act as an autoantigen and appears to be immunogenic only for a very limited number of selected patients.

TABLE 2
Antigen-specific T-cell stimulation: Analysis of the ELISPOT results by t-Test p values

| | | | | HLA-Cw6-positive |
|---|---|---|---|---|
| | | HLA-Cw6-negative | HLA-Cw6-positive | versus |
| | Psoriasis versus | Psoriasis versus | Psoriasis versus | HLA-Cw6-negative |
| Protein | healthy individuals | healthy individuals | healthy individuals | Psoriasis |
| Heat shock protein beta-1 | 0.0708 | 0.0307 | 0.3274 | 0.0729 |
| Ezrin | 0.0600 | 0.0681 | 0.1569 | 0.3886 |
| Serpin B5 | 0.0091 | 0.3768 | 0.0026 | 0.0119 |
| Peroxiredoxin-2 | 0.0003 | 0.0301 | 0.0006 | 0.1177 |
| K6-N | 0.2242 | 0.3572 | 0.1587 | 0.3440 |
| K6-C | 0.4581 | 0.3397 | 0.3171 | 0.2572 |
| Cytokeratin | 0.0641 | 0.1335 | 0.0468 | 0.2802 |

5. Fragment Length Analysis

Our data demonstrate a particular antigenicity of peroxiredoxin-2, maspin, Hsp27, and potentially ezrin, for the T cells of psoriasis patients. T cells recognize antigen peptides presented by MHC-molecules by means of their TCRs. TCRs are heterodimers composed of an α- and β-chain. Antigen-specificity of each of these chains is defined by the complementarity determining region 3 (CDR3). It results from the recombination of one of several variable (V) genes with one diversity (D) and one of several joining genes (J). Variability of the VDJ recombination is enhanced further by random deletion or addition of nucleosides at the recombination sites. This creates a tremendous variability making the TCR a distinctive attribute for each T cell and its clonal progenies.

Stimulation of PBMCs with antigen may promote the expansion of antigen-specific T cells and generate oligoclonal T-cell populations that can be identified by a restricted TCR usage. Moreover, identical or similar amino acid compositions of the CDR3 may denote T cells with specificity for the same antigenic peptides. We employed these attributes of T-cell antigen recognition to further characterize the relevance of the potential autoantigens for the psoriatic immune response of a 23-year old patient with severe streptococcal-driven type-1 psoriasis.

Antigen-specific T-cell lines were generated in vitro by periodic restimulation of the patient's PBMC with the different recombinant proteins except keratin 6 that had not given a sufficient response. By TCR-fragment length analysis and sequencing of TCR β-chain rearrangements their TCR usage was compared to that of the patient's skin lesion, blood lymphocytes and control T-cell lines. For each TCR β-chain repertoire the cDNA from the different samples was amplified by PCR using 26 different primers specific for the TCRBV gene families 1-24 together with a dye-labelled TCRBC-specific primer. The spectratypes of fragment lengths of the amplified TCR β-chain rearrangements were determined on a genetic sequencer. This approach may identify clonal T-cell expansions within a given TCRBV gene family by a biased usage of TCR lengths.

TCR fragment length spectratyping of non-stimulated PBMC and of the PHA-driven T-cell lines showed a predominance of quasi-Gaussian repertoire β-chain lengths in most TCRBV-gene families and reflected largely unselected T-cell populations. Instead, several TCRBV-gene spectratypes of the antigen-specific T-cell lines generated from the patient's PBMC and of the psoriatic skin lesion displayed a highly restricted pattern of fragment lengths, with discrete prominent peaks suggesting oligo-clonal antigen-driven T-cell expansion. When compared to each other several of the biased TCRBV-gene spectratypes of the antigen-specific T-cell lines and the psoriatic skin lesion displayed select prominent peaks of identical fragment length (see Table 3). These data suggested that within both the psoriatic skin lesion and the antigen-specific T-cell lines T cells had been selected that shared TCR beta-chains of identical length.

The cDNA of these TCRBV-gene families was cloned and sequenced. TCR rearrangements of the blood T cells and the PHA-driven T-cell line were clearly heterogeneous. Instead, many of the TCR rearrangements of the antigen-specific T-cell lines were highly repetitive. Individual clonal TCR rearrangements represented up to 93% of the analysed TCR sequences of a given TCRBV-gene family. These data strongly emphasized that T-cell expansion within the T-cell lines had been driven by the respective keratinocyte protein in an antigen-specific manner. A similar clonal dominance of particular TCR rearrangements was seen within the psoriatic skin lesion. This corroborated former findings that T-cell activation in psoriasis occurs in response to defined antigens.

TABLE 3
TCR-β-chain families showing dominant peaks of identical
size in psoriatic skin lesions and antigen-specific T-cell lines

| | | Heat shock | | Peroxiredoxin- |
|---|---|---|---|---|
| TCRBV | Ezrin | protein beta-1 | Serpin B5 | 2 |
| 3 | | xx | | xx |
| 6 | xx | | | |
| 8 | | | xx | xx |
| 9 | | | xx | |
| 13.1 | x | | | |
| 13.2 | | | | xxx |
| 14 | xx | | | |
| 17 | | | xx | |
| 21 | | | xx | xx |

XX = selection of T-cell receptor β-chain rearrangements of identical length in antigen-specific T-cell line and psoriatic skin lesion

6. Comparison of the CDR3 β-Motifs

The role of the keratinocyte proteins as psoriatic autoantigens might be reflected by homologies in the selected TCR β-chain CDR3s of the antigen-specific T-cell lines and the psoriatic skin lesion. Therefore, the deduced amino acid sequences of the TCR β-chain rearrangements from the different sources were compared to each other.

Several CDR3-β chain motifs of the clonally selected TCR rearrangements of the antigen-specific T-cell lines and the lesional psoriatic infiltrate were highly homologous (Tables 4-6). A preferred CDR3 amino acid sequence of the Peroxiredoxin-2-specific T-cell line, SSGTG, was found in several modifications within the psoriatic skin lesion, sharing up to five amino acids in homology. A selected CDR3 motif of the ezrin-specific T-cell line, SSSGS, was found in two variations, SSSG and LSSG, in the skin lesion.

The Serpin B5-specific and the ezrin-specific T-cell lines shared a variation of a dominant CDR3 motif, (F/G/P)LAG(G/V) with the psoriatic skin lesion. Up to five amino acids were identical. Most interestingly, this amino acid sequence variation had recently been identified as a conserved CDR3 motif preferentially selected within psoriatic skin lesions of identical twins concordant for psoriasis and other psoriasis patients with type 1 psoriasis. No similar degree of homologies was observed in the corresponding blood sample.

Thus, several apparent homologies in the CDR3 of TCR β-chain rearrangements within the antigen-specific T-cell lines and the psoriatic skin lesions support a role for the corresponding keratinocyte proteins ezrin, serpin B5, and peroxiredoxin-2 as psoriatic autoantigens. The common usage of the (G)LAG(G)-motif by the Serpin B5- and ezrin-specific T-cell lines furthermore suggests that both antigens, although showing no apparent sequence homologies at amino acid level, may contain epitopes engaging similar TCR rearrangements.

The (G)LAG(G) CDR3 motifs expanded within the psoriatic skin lesion and the T-cell lines was similar to a conserved CDR3 motif formerly identified in psoriatic skin lesions of identical twins with psoriasis and other psoriasis patients (see Table 7). This corroborates the antigenic relevance of the corresponding keratinocyte proteins further.

TABLE 4
Homologies of the CDR3 of selected TCR β-chain
rearrangements of the Peroxiredoxin-2-specific
T-cell line and the psoriatic skin lesion

| Source | TCR | BV | N-D-N | BJ | | id./sequ | % |
|---|---|---|---|---|---|---|---|
| Peroxi | BV3 | CA [symbol] | [symbol] | ETQYFG | 2.5 | 10/36 | 27.7 |
| redoxin-2- | | CA [symbol] | [symbol] | QETQYFG | 2.5 | 2/36 | 5.6 |
| specific | | CAS [symbol] | [symbol] | ETQYFG | 2.5 | 1/36 | 2.8 |
| TCL | | CASS | [symbol] | QETQYFG | 2.5 | 1/36 | 2.8 |
| | BV8 | CASSL | I [symbol] SD | EQFFG | 2.1 | 22/63 | 34.9 |
| | | CASSL | IT [symbol] PSD | EQFFG | 2.1 | 1/63 | 1.6 |
| | | CASS | IT [symbol] D | EQYFG | 2.7 | 1/63 | 1.6 |
| Psoriatic | BV8 | CASSL | F [symbol] SSRGAEHK | TQYFG | 2.5 | 2/33 | 18.1 |
| skin | | CASS | R [symbol] VW | EQYFG | 2.7 | 1/33 | pooled |
| lesion | | CASS | A [symbol] NV | NEQFFG | 2.1 | 1/33 | |
| | | CAS [symbol] | [symbol] D | SGANVLTFG | 2.6 | 1/33 | |
| | | CASS | [symbol] VW | EQYFG | 2.7 | 1/33 | |
| | BV3 | CAS | RRK [symbol] T | YEQYFG | 2.7 | 16/38 | 42.1 |
| | | CAS | GRK [symbol] T | YEQYFG | 2.7 | 1/38 | |
| | BV14 | ASSL | [symbol] VY | YEQYFG | 2.7 | 5/29 | 17.2 |

TABLE 5
Homologies of the CDR3 of selected TCR β-chain
rearrangements of the Ezrin-specific T-cell
line and the psoriatic skin lesion

| | | | | | | id./ | |
|---|---|---|---|---|---|---|---|
| Source | TCR | BV | N-D-N | BJ | | sequ. | % |
| ezrin- | BV3 | CAS | [symbol] S | YNEQFFG | 2.1 | 13/38 | 34.2 |
| specific | | | | | | | |
| TCL | | | | | | | |
| Psoriatic | BV13S1 | CASSY | [symbol] | NTEAFFG | 1.1 | 7/37 | 18.9 |
| skin | | | | | | | |
| lesion | | CASSY | LS [symbol] | NTEAFFG | 1.1 | 2/37 | |

TABLE 6
Homologies of the CDR3 of selected TCR β-chain
rearrangements of the Serpin B5-specific and the
CSLP-specific T-cell line and the psoriatic skin lesion

| Source | TCR | BV | N-D-N | BJ | | id./sequ. | % |
|---|---|---|---|---|---|---|---|
| Serpin B5- | BV9 | CASS | [symbol] | SYNEQFFGPGT | 2.1 | 6/15 | 40.0 |
| specific TCL | | | | | | | |
| Ezrin-specific | BV13S1 | CASS | F [symbol] P | NEQFFG | 2.1 | 12/40 | 30.0 |
| TCL | | CASS | F [symbol] L | NEQFFG | 2.1 | 1/40 | |
| | | CASS | PLRA [symbol] | TDTQYFG | 2.3 | 2/40 | 5.0 |
| Psoriatic skin | BV 3 | CASS | [symbol] RG | STDTQYFG | 2.3 | 8/38 | 20.5 |
| lesion | BV13S1 | CASS | YG [symbol] | TGELFFG | 2.2 | 5/37 | 13.5 |
| | | CASS | G [symbol] | YNEQFFG | 2.1 | 4/37 | 10.8 |
| | | CAS | S [symbol] | SYNEQFFG | 2.1 | 1/37 | |
| | | CASS | YH [symbol] GSG | ETQYFG | 2.5 | 4/37 | 10.8 |
| | BV14 | CAS | RLL [symbol] E | YNEQFFG | 2.1 | 2/37 | |
| | BV8 | CASS | [symbol] PS | YNEQFFG | 2.1 | 1/63 | |
| | BV14 | CASS | S [symbol] Q | ETQYFG | 2.5 | 3/29 | 10.3 |
| | BV21 | CASS | [symbol] | ETQYFG | 2.5 | 1/19 | |
| | | CASS | S [symbol] | ETQYFG | 2.5 | 1/19 | |

TABLE 7
Conserved lesional psoriatic CDR3 β-chain
(G)LAG(G/S) rearrangements in former studies
(references are: #1: Prinz et al., Eur. J.
Immunol. 1999; 20:3360-3368; and #2: Chang et
al. Proc. Natl. Acad. Sci. USA 1994; 91:
9282-9286)

| Ref. | Patient | TCR | BV | N-D-N | BJ |
|---|---|---|---|---|---|
| #1 | D.K. | BV6 | CASS | [symbol] | QETQYFG |
| | | BV9 | CASSQ | DS [symbol] S | SYEQYFG |
| | K.K. | BV6 | CASSL | A [symbol] | TDTQYFG |
| | | BV9 | CASSQ | DS [symbol] S | SYEQYFG |
| | | | CASS | MW [symbol] APS | SYEQYFG |
| | | | CASS | L [symbol] N | EQYFG |
| | H.W. | BV2 | CSA | R [symbol] AL | DTQYFG |
| | P.A. | BV6 | CASS | R [symbol] E | YEQYFG |
| | A.S. | BV6 | CASS | S [symbol] S | YEQYFG |
| | B.S. | BV20 | CAWS | [symbol] RD | YNEQFFG |
| #2 | | BV3 | CASS | PHVLA [symbol] ASGG | YNEQFFG |
| | | BV13S1 | CA | T [symbol] GTGMRN | EQYFG |

### Example 2

1. Stimulatory Properties of Candidate Peptides

According to the hypothesis of molecular mimicry the putative psoriatic autoantigens became targets of the pathogenic psoriatic immune response due to amino acid sequence homologies with streptococcal proteins. In order to determine whether homologous regions of the keratinocyte proteins and streptococcal antigens would be able to stimulate T cells from psoriasis patients on the peptide level, several candidate peptides selected on the basis of sequence similarities were arbitrarily chosen. They are listed in Table 8. To identify these candidate epitopes the amino acid sequence of the four keratinocyte proteins were searched against the sequence data of Streptococcus pyogenes. The homologies were identified by BLAST-searches at the NCBI server against Streptococcus pyogenes, Serotyp M1, TaxID 301447, or Streptococcus pyogenes, TaxID 1314. The latter includes all known sequences of Streptococcus pyogenes.

The BLAST-searches represent a comparison for homology between the proteins or deduced amino acid sequences of the genome of Streptococcus pyogenes. The queries were performed as protein-protein searches for short nearly exact sequence homologies. The used matrix was PAM30 without compositional adjustment. Word size was set to two.

2. Peptide Stimulation

In a first approach, peripheral blood lymphocytes from 32 psoriasis patients and 17 healthy controls were stimulated in triplicates in vitro with synthetic peptides (10 μg/ml) corresponding to the sequences chosen (Table 8). They were either derived from the keratinocyte proteins or from streptococcal antigens. After five days of stimulation peptide-induced proliferation was determined by ³H-thymidine (³H-TdT) incorporation for eight hours and measured as counts per minute (cpm). For statistical analysis means of cpm were compared in f- and t-tests. Probability of error (p value) was set to p<0.05.

Several of the peptides induced an increased T-cell proliferation in psoriasis patients as compared to the healthy controls. This difference was statistically significant for the keratinocyte peptides PRDX2 pep 2, Hsp b1 pep 2, Erzrin pep 2, Serpin B5 pep 1 and pep 2, and Serpin B5/Strep, and for the streptococcal peptide RopA (Table 9).

For 25 of these patients HLA-haplotypes had been determined. When the results were differentiated according to the HLA-haplotypes, HLA-Cw6 positive (n=16) but not HLA-Cw6 negative patients (n=9) showed a significantly increased response to PRDX2 pep 2, Hsp b1 pep 2, Ezrin pep 2, and to the streptococcal peptides Serpin B5/Strep, RopA and RecF.

These data demonstrate that certain defined peptides chosen from the four keratinocyte autoantigens according to amino acid sequence homologies with streptococcal proteins can stimulate the peripheral blood lymphocytes from psoriasis patients to a greater extend than the peripheral blood lymphocytes from healthy controls. For several of these peptides the stimulatory capacity is particularly evident in HLA-Cw6-positive patients.

3. ELISPOT Analysis

In a second approach the ability of defined peptides [10 μg/ml] to stimulate the peripheral blood lymphocytes of 18 HLA-Cw6 positive psoriasis patients was compared to stimulation with tetanus toxoid [5 μg/ml], a common nominal antigen used for vaccination against tetanus, or to stimulation with phytohaemagglutinin (PHA, diluted 1:100). T-cell stimulation was measured in an ELISPOT assay identifying IFN-γ producing cells. Results are given as spot forming colonies (SFCs) per 1.5×10⁵ PBMC after subtraction of the corresponding negative background values.

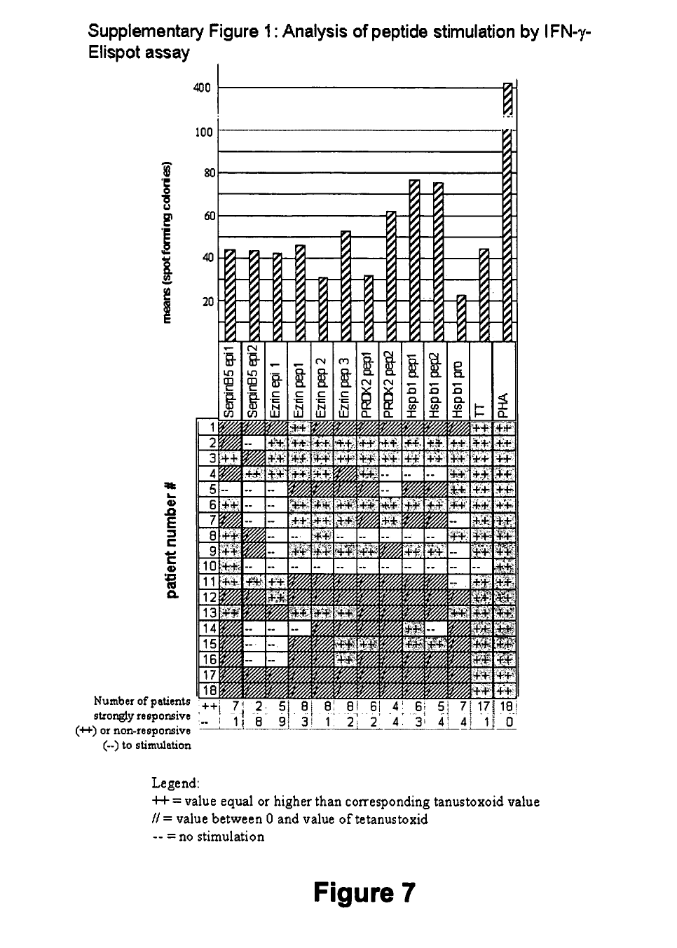

The results are shown in FIG. 7. PHA, which served as positive control for the ability to secrete IFN-γ, induced a strong stimulation of peripheral blood lymphocytes in all patients. Tetanus toxoid stimulation served as a reference for the magnitude of an antigen-specific T-cell stimulation. Only one of the patients (#10) did not respond to it. All patients responded to stimulation with at least one of the different peptides from the potential autoantigens, although different patterns of response were seen. On average, the mean level of stimulation with peptides was similar or even greater than the stimulation induced by tetanus toxoid.

These data demonstrate that several peptides may stimulate the peripheral blood lymphocytes of psoriasis patients, and that this stimulatory capacity may be comparable or even greater than the stimulation achieved by tetanus toxoid, which is a nominal antigen reflecting the vaccination status against tetanus.

4. Results

Together the data from peptide stimulation demonstrate that peptides corresponding to homologous regions of streptococcal proteins from the four potential autoantigens peroxiredoxin 2, serpin B5, heat shock protein beta-1 (hsp 27), and ezrin represent antigens for the T-cell mediated immune response in psoriasis patients. Furthermore they support that these particular protein regions can indeed constitute epitopes for a cross-reactive autoimmune response induced by infection with Streptococcus pyogenes. Thus, they exemplarily stand for molecular mimicry as a mechanism for streptococcal-induced autoimmunity.

TABLE 8
Explanation of peptides from the potential autoantigens Peroxiredoxin 2,
Heat shock protein beta-1, Ezrin, and Serpin B5, and definition of
amino acid sequence homologies (one letter code) as determined by
homology searches with proteins from Streptococcus pyogenes, or vice versa

| | | Amino acid | | |
|---|---|---|---|---|
| | | sequence (one | | |
| Abbreviation | Full name | letter code) | Position | Homology to |
| Keratinocyte | | | | |
| peptides | | | | |
| Serpin B5 epi 1 | Serpin B5 epitope 1 | YSLKLIKRL | 84-92 | |
| SEQ ID No. 233 | | | | |
| SEQ ID No. 234 | | YS KLIK L | | |
| SEQ ID No. 235 | | YS-KLIKHL | 43-50 | ftsH cell division |
| | | | | protein |
| SEQ ID No. 236 | Serpin B5 epitope 1 | YSLKLIKRL | 84-92 | |
| SEQ ID No. 237 | | Y+LK+IK | | |
| SEQ ID No. 238 | | YALKIIK | 129-135 | dipeptidase PepV |
| Serpin B5 epi2 | Serpin B5 epitope 2 | GLEKIEKQL | 242-250 | |
| SEQ ID No. 239 | | | | |
| SEQ ID No. 240 | | GLEKIE | | |
| SEQ ID No. 241 | | GLEKIE | 347-352 | putative surface- |
| | | | | anchored protein |
| SEQ ID No. 242 | Serpin B5 epitope 2 | GLEKIEKQL | 242-250 | |
| SEQ ID No. 243 | | LE+IEKQ | | |
| SEQ ID No. 244 | | LEEIEKQ | 446-452 | Mga |
| | | | | [Streptococcus |
| | | | | pyogenes] |
| Serpin B5 pep1 | SerpinB5 peptide 1 | FCMGNIDSINCK | 204-216 | |
| SEQ ID No. 245 | | | | |
| SEQ ID No. 246 | | NIDS | | |
| SEQ ID No. 247 | | NIDS | 464-467 | ScnM |
| | | | | [Streptococcus |
| | | | | pyogenes] |
| Ezrin epi | Ezrin epitope | EYTAKIAEL | 423-431 | |
| SEQ ID No. 248 | | | | |
| SEQ ID No. 249 | | E TAKIAL | | |
| SEQ ID No. 250 | | EVTAKIAL | 174-181 | putative GTP |
| | | | | pyrophosphokinase |
| SEQ ID No. 251 | Ezrin epitope | EYTAKIAEL | 423-431 | |
| SEQ ID No. 252 | | EY AKIA | | |
| SEQ ID No. 253 | | EYNAKIA | 207-213 | M protein |
| | | | | precursor |
| | | | | [Streptococcus |
| | | | | pyogenes] |
| Ezrin pep 1 | Ezrin peptide 1 | LSSELSQAR | 534-542 | |
| SEQ ID No. 254 | | | | |
| SEQ ID No. 255 | | SEL+QAR | | |
| SEQ ID No. 256 | | SELTQAR | 233-239 | immunoglobulin- |
| | | | | Fc-binding protein |
| | | | | [Streptococcus |
| | | | | pyogenes] |
| SEQ ID No. 257 | Ezrin peptide 1 | LSSELSQAR | 534-542 | |
| SEQ ID No. 258 | | LSSELS | | |
| SEQ ID No. 259 | | LSSELS | 51-56 | 3-ketoacyl- |
| | | | | reductase |
| | | | | [Streptococcus |
| | | | | pyogenes |
| Ezrin pep 2 | Ezrin peptide 2 | LNIYEKDDKL | 225-234 | |
| SEQ ID No. 260 | | | | |
| SEQ ID No. 261 | | LNI+E DKL | | |
| SEQ ID No. 262 | | LNIFESQDKL | 242-251 | fibronectin-binding |
| | | | | protein |
| | | | | [Streptococcus |
| | | | | pyogenes] |
| SEQ ID No. 263 | Ezrin peptide 2 | LNIYEKDDKL | 225-234 | |
| SEQ ID No. 264 | | +IYEKD | | |
| | | | 294-299 | Cell division |
| | | | | protein ftsY |
| Ezrin pep 3 | Ezrin peptide 3 | AKEELERQA | 399-407 | |
| SEQ ID No. 265 | | | | |
| SEQ ID No. 266 | | AKEELE+Q | | |
| SEQ ID No. 267 | | AKEELEKQ | 552-559 | Streptococcus |
| | | | | pyogenes |
| | | | | MGAS10750] |
| SEQ ID No. 268 | Ezrin peptide 3 | AKEELERQA | | |
| SEQ ID No. 269 | | EELERQ | | |
| SEQ ID No. 270 | | EELERQ | 132-137 | M protein |
| | | | | [Streptococcus |
| | | | | pyogenes] |
| SEQ ID No. 271 | | AKEELERQA | | |
| SEQ ID No. 272 | | KE+ ERQ | | |
| SEQ ID No. 273 | | KEQQERQ | 122-128 | M protein |
| | | | | [Streptococcus |
| | | | | pyogenes] |
| PRDX2 pep1 | Peroxiredoxin 2 peptide | AFKEVKLSDYKG | 24-35 | |
| SEQ ID No. 274 | 1 | | | |
| SEQ ID No. 275 | | EVKL DYK | | |
| SEQ ID No. 276 | | EVKLGDYK | 144-151 | trigger factor |
| | | | | [Streptococcus |
| | | | | pyogenes |
| | | | | MGAS10270] |
| SEQ ID No. 277 | | AFKEVKLSDYKG | | |
| SEQ ID No. 278 | | A K+ KLS DY | | |
| | | ALKQAKLS DY | 98-107 | recombination |
| | | | | protein F |
| | | | | [Streptococcus |
| | | | | pyogenes M1 |
| | | | | GAS] |
| PRDX2 pep2 | Peroxiredoxin 2 peptide | EVKLSDYKGKYV | 27-38 | |
| SEQ ID No. 279 | 2 | | | |
| SEQ ID No. 280 | | EVKL DYK | | |
| SEQ ID No. 282 | | EVKLGDYK | 144-151 | trigger factor, |
| | | | | PPlase |
| SEQ ID No. 283 | | EV---KL--- | 27-38 | |
| | | SDYKGKY | | |
| SEQ ID No. 284 | | EV KL | | |
| | | SDYKGKY | | |
| SEQ ID No. 285 | | EVEIPKLAFPSDYKG | 87-103 | 23S rRNA |
| | | KY | | methyltransferase |
| HspB1 pep1 | heat shock protein beta- | SEIRHTADRWRVSL | 86-99 | |
| SEQ ID No. 286 | 1 peptide 1 | | | |
| SEQ ID No. 287 | | SEI H ADR | | |
| SEQ ID No. 288 | | SEIEHIADR | 104-202 | ScnF |
| | | | | [Streptococcus |
| | | | | pyogenes] |
| SEQ ID No. 289 | | SEIRHTADRWRVSL | | |
| SEQ ID No. 290 | | D+WR SL | | |
| SEQ ID No. 291 | | DKWRASL | 98-104 | collagen-like |
| | | | | protein SclB |
| HspB1 pep 2 | heat shock protein beta- | QLSSGVSEIRH | 80-90 | |
| SEQ ID No. 292 | 1 peptide 2 | | | |
| y SEQ ID No. 293 | | LS GVSE | | |
| SEQ ID No. 294 | | HLSGGVSE | 243-250 | ScnT |
| | | | | [Streptococcus |
| | | | | pyogenes] |
| SEQ ID No. 295 | heat shock protein beta- | QLSSG-VSEIRH | 80-90 | |
| | 1 peptide 2 | | | |
| SEQ ID No. 296 | | QL G V+EIRH | | |
| SEQ ID No. 297 | | QLGGGKVTEIRH | 286-297 | Na+ driven |
| | | | | multidrug efflux |
| | | | | pump |
| | | | | [Streptococcus |
| | | | | pyogenes] |
| SEQ ID No. 298 | | QLSSGVSEIRH | | |
| SEQ ID No. 299 | | SEIRH | | |
| SEQ ID No. 300 | | SEIRH | 284-288 | putative ABC |
| | | | | transporter ATP- |
| | | | | binding protein |
| SEQ ID No. 301 | | QLSSGVSEIRH | | |
| SEQ ID No. 302 | | LSSG | | |
| SEQ ID No. 303 | | LSSG | 56-59 | putative ABC |
| | | | | transporter ATP- |
| | | | | binding protein |
| SEQ ID No. 304 | | QLSSGVSEIRH | | |
| SEQ ID No. 305 | | LS VSEI+H | | |
| SEQ ID No. 306 | | LS--VSEIQH | 309-316 | polysaccharide |
| | | | | deacetylase family |
| | | | | protein |
| SEQ ID No. 307 | | QLSSGVSEIRH | | |
| SEQ ID No. 308 | | SSGVS I | | |
| SEQ ID No. 309 | | SSGVSAI | 473-479 | hypothetical |
| | | | | membrane |
| | | | | associated protein |
| HspB1 pro | heat shock protein beta- | full length | | |
| SEQ ID No. 310 | 1 | protein | | |
| Streptococcal | | | | |
| peptides | | | | |
| RecF | recombination protein F | ALKQAKLSDYIG | 98-109 | |
| SEQ ID No. 311 | of S.pyogenes | | | |
| SEQ ID No. 312 | | A K+ KLSDY G | | |
| SEQ ID No. 313 | | AFKEVKLSDYKG | 24-35 | PRDX2 pep1 |
| RopA | trigger factor | EVKLGDYKNLVV | 144-155 | |
| SEQ ID No. 314 | S.pyogenes PPlase | | | |
| SEQ ID No. 315 | | EVKL DYK V | | |
| SEQ ID No. 316 | | EVKLSDYKGKYV | 27-38 | PRDX2pep2 |
| Strepl/Hsp b1 | S.pyogenes ScnF | SEIEHIADRVGIIN | 194-207 | |
| SEQ ID No. 317 | (homologous to Hsp b1) | | | |
| | gb\|AAB92604.1\| | | | |
| SEQ ID No. 318 | | SEI H ADR | | |
| SEQ ID No. 319 | | SEIRHTADRWRVSL | 86-99 | HspB1 pep1 |
| Strep2/Hsp b1 | S.pyogenes ScnF | ISSQILSEIEH | 188-198 | |
| SEQ ID No. 320 | (homologous to Hsp b1) | | | |
| | gb\|AAB92604.1\| | | | |
| SEQ ID No. 321 | | SS SEI H | | |
| SEQ ID No. 322 | | QLSSGV SEIRH | 80-90 | HspB1 pep 2 |
| Strep/Serpin B5 | S.pyogenes ScnM | YFYSNIDSCDIK | 460-471 | |
| SEQ ID No. 323 | (homologous to Serpin | | | |
| | B5) gb\|AAB92602.1\| | | | |
| SEQ ID No. 324 | | NIDS | | |
| SEQ ID No. 325 | | FCMGNIDSINCK | 204-216 | SerpinB5 Pep |

* = used sequence, original: EYTAKIALL

1-40. (canceled)

41. A method for the detection of a streptococcal driven condition comprising detecting the presence of a humoral and/or a cellular immune response against at least one autoantigen selected from Ezrin, Serpin B5, Peroxiredoxin-2, heat shock protein β1, and/or peptides comprising at least 5 consecutive amino acid residues of one of these autoantigenic proteins having immunological activity, or fragments, variants, or epitopes thereof and/or mixtures of at least two of the proteins and/or peptides in a sample.

42. The method according to claim 41 for detecting the presence of a humoral and/or a cellular immune response in a patient to at least one of the autoantigens as defined in claim 41, the method comprising the following steps:
(a) obtaining or providing a sample from a patient;
(b) detecting a humoral immune response against at least one of the autoantigens of claim 41 by qualitatively and/or quantitatively detecting in the sample at least one antibody against one or more of the autoantigenic proteins Ezrin, Serpin B5, Peroxiredoxin-2 and heat shock protein beta-1, or a fragment, variant or epitope thereof, using biophysical or biomolecular detection methods; and/or
(c) detecting a cellular immune response against at least one of the autoantigens of claim 41 in the sample by qualitatively and/or quantitatively detecting at least one factor involved with a cellular immune response using biophysical or biomolecular detection methods;
(d) evaluating the results of step (b) and/or step (c).

43. The method of claim 41 for detecting a streptococcal driven condition by qualitatively and/or quantitatively determining the presence of at least one antibody against at least one of the autoantigenic proteins Ezrin, Serpin B5, Peroxiredoxin-2 and Heat shock protein beta-1 in a sample, or a fragment, variant or epitope thereof, the method comprising the following steps:
(a) obtaining or providing a sample from a patient or a synthetic or natural source putatively containing at least one antibody against one or more of the autoantigenic proteins Ezrin, Serpin B5, Peroxiredoxin-2 and heat shock protein beta-1, or a fragment, variant or epitope thereof;
(b) contacting the sample with an autoantigen as defined in claim 1 to allow binding of antibodies present in the sample with the at least one autoantigen and
(c) qualitatively and/or quantitatively determining the presence of the at least one antibody using biophysical or biomolecular detection methods.

44. The method of claim 41, wherein the streptococcal driven condition is an autoimmune condition.

45. The method of claim 41, wherein the condition is selected from the group consisting of psoriasis, including psoriasis vulgaris (plaque psoriasis), erythrodermic psoriasis, pustular psoriasis, psoriatic arthritis, and autoimmune disorders including rheumatic fever and heart disease, post-streptococcal glomerulonephritis, and pediatric autoimmune neuropsychiatric disorders associated with streptococcal infections (PANDAS).

46. The method of claim 41, wherein the at least one autoantigen is selected from a sequence comprising any of SEQ ID NO: 1, 2, 3 or 4, or a sequence having an identity of at least about 60%, preferably of at least about 70% or about 80%, even more preferably of at least about 90% or about about 95%, and most preferably an identity of at least about 99% with a sequence according to SEQ ID NO: 1, 2, 3 or 4.

47. The method of claim 41, wherein the epitope has about 5 to 35 amino acids.

48. The method of claim 41, wherein the at least one autoantigenic protein is selected from an epitope comprising a sequence according to any of SEQ ID NOs: 5 to 83 (Ezrin), any of SEQ ID NOs: 84 to 144 (Serpin B5), any of SEQ ID NOs: 145 to 203 (Peroxiredoxin-2) and/or any of SEQ ID NOs: 204 to 232 (Heat shock protein beta-1).

49. The method of claim 41, wherein the at least one autoantigenic protein carries a peptide, group or a linker for immobilization on a solid phase, which allows binding of the at least one autoantigenic protein to a sample carrier, support, or matrix.

50. The method of claim 41 for detecting the presence of a humoral and/or a cellular immune response in a patient suffering from diseases induced and/or mediated by group A beta-haemolytic streptococci (GAS).

51. A method for treating streptococcal driven conditions wherein an autoantigen selected from Ezrin, Serpin B5, Peroxiredoxin-2, heat shock protein β1, and/or a peptide comprising at least 5 consecutive amino acid residues of one of these autoantigenic proteins having immunological activity, or fragments, variants, or epitopes thereof and/or mixtures of at least two of the proteins and/or peptides and/or an antibody or antibody fragment directed against at least one of the above autoantigens is administered to a patient in need thereof.

52. The method of claim 51 wherein the streptococcal driven disease is an autoimmune disease.

53. The method of claim 51, wherein the treatment is hyposensitization or desensitization of an autoimmune disease.

54. The method of claim 53, wherein the treatment is directed to desensitizing or hyposensitizing the immune system of a patient to be treated with respect to excessive or exaggerated immune reactions.

55. The method of claim 51, wherein the condition is selected from the group consisting of psoriasis, including psoriasis vulgaris (plaque psoriasis), erythrodermic psoriasis, pustular psoriasis, psoriatic arthritis, and autoimmune disorders including rheumatic fever and heart disease, post-streptococcal glomerulonephritis, and pediatric autoimmune neuropsychiatric disorders associated with streptococcal infections (PANDAS).

56. The method of claim 51, wherein the at least one autoantigen is selected from a sequence comprising any of SEQ ID NO: 1, 2, 3 or 4, or a sequence having an identity of at least about 60%, preferably of at least about 70% or about 80%, even more preferably of at least about 90% or about about 95%, and most preferably an identity of at least about 99% with a sequence according to SEQ ID NO: 1, 2, 3 or 4.

57. The method of claim 51, wherein the epitope has about 5 to 35 amino acids.

58. The method of claim 51, wherein the at least one autoantigenic protein is selected from an epitope comprising a sequence according to any of SEQ ID NOs: 5 to 83 (Ezrin), any of SEQ ID NOs: 84 to 144 (Serpin B5), any of SEQ ID NOs: 145 to 203 (Peroxiredoxin-2) and/or any of SEQ ID NOs: 204 to 232 (Heat shock protein beta-1).

59. A pharmaceutical composition, comprising:
(a) at least one autoantigen as defined in claim 41.; and/or
(b) at least an antibody directed against an autoantigen as defined in claim 41 and/or
(c) an anti-idiotypic antibody directed against an antibody of (b); and
(d) a pharmaceutically acceptable carrier, exipient, adjuvant, and/or vehicle.

60. The pharmaceutical composition of claim 59, which is a vaccine for desensibilization or hyposensibilization.

61. A test kit comprising
(a) at least one autoantigen as defined in claim 41, optionally immobilized on a carrier;
(b) a container for receiving a sample to be analyzed and optionally
(c) carriers, adjuvants and/or excipients, and a buffering agent for adapting the pH.

62. A kit comprising a pharmaceutical composition according to claim 59, and technical instructions with information on the administration and dosage of these components.

63. A kit comprising a pharmaceutical composition according to claim 60, and technical instructions with information on the administration and dosage of these components.

64. An anti-idiotypic antibody, directed against an antibody that is directed against an autoantigen as defined in claim 41.
